(19) United States
(12) Patent Application Publication
(10) Pub. No.: US 2021/0228494 A1
(43) Pub. Date: Jul. 29, 2021

(54) DRUG DELIVERY SYSTEMS AND METHODS COMPRISING POLYSIALIC ACID AND/OR OTHER POLYMERS

(71) Applicant: Universidade de Santiago de Compostela, Santiago de Compostela (ES)

(72) Inventors: María José Alonso Fernandez, Santiago de Compostela (ES); Desireé Teijeiro Osorio, Santiago de Compostela (ES); Carmen María Teijeiro Valiño, Santiago de Compostela (ES); Ana Cadete Pires, Santiago de Compostela (ES)

(73) Assignee: Universidade de Santiago de Compostela, Santiago de Compostela (ES)

(21) Appl. No.: 16/760,527

(22) PCT Filed: Nov. 2, 2018
(86) PCT No.: PCT/EP2018/080050

§ 371 Date: Apr. 30, 2020

(30) Foreign Application Priority Data
Nov. 2, 2017 (ES) P201731277

(51) Int. Cl. A61K 9/51; C07K 16/22; A61K 39/395; A61K 47/62; A61K 47/69

(52) U.S. Cl. CPC A61K 9/5161; C07K 16/22; A61K 39/3955; B82Y 5/00; A61K 47/62; A61K 47/6925; A61K 9/5192

(57) ABSTRACT

The present invention generally relates to particles, including nanocapsules or other nanoentities, comprising a polymer such as polysialic acid. The particles are able to access the interior of the cells, and/or to procure the intracellular release of the associated drugs. In 5 one aspect, the present invention is directed to nanocapsules or other entities having an exterior or surface comprising a polymer such as polysialic acid. In some cases, targeting moieties such as Lyp-1 or tLyp-1 peptide are bonded to the polymer, e.g., using aminoalkyl (C₁-C₄) succinimide or other linkers. These may be created, for example, by reacting a carboxylate moiety on a polymer with an aminoalkyl maleimide (C₁-C₄) or an aminoalkyl 10 (C₁-C₄) methacrylamide, and reacting the resulting aminoalkyl (C₁-C₄) maleimide or the aminoalkyl (C₁-C₄) methacrylamide to a cysteine or other sulfur group. Targeting moieties are bonded to the polymer, for example, by reacting a carboxylate moiety on a polymer with a N-hydroxysuccinimide or a carbodiimide, and reacting the intermediate formed with a lysine or arginine group on a targeting peptide to produce polymer-amide-peptide. Other 15 aspects of the invention are generally directed to methods of making or using such compositions, kits including such compositions, or the like.

### RELATED APPLICATIONS

This application is a national stage filing under 35 U.S.C. § 371 of International Patent Application Serial No. PCT/EP2018/080050, filed Nov. 2, 2018, entitled “Drug Delivery Systems and Methods Comprising Polysialic Acid and/or Other Polymers,” which claims priority to Spanish Application Serial No. P201731277, filed Nov. 2, 2017, entitled “Sistemas de Liberatión de Fármacos de Ácido Polisiálico y Métodos.” Each of these applications is incorporated herein by reference in its entirety.

### FIELD OF THE INVENTION

The present invention generally relates to particles, including nanocapsules or other nanoentities, comprising polymers such as polysialic acid, for acting as carriers to deliver drugs or other active substances internally into cells, or other applications.

### BACKGROUND ART

The targeted delivery of pharmaceutical agents into the body has been an ongoing challenge. For example, many drugs cannot effectively exert their action because of their difficult access to target cells.

Thus, improvements in the delivery of pharmaceutical agents, are needed.

### SUMMARY OF THE INVENTION

The present invention generally relates to particles, including nanocapsules or other nanoentities, comprising polymers such as polysialic acid (hereinafter “PSA”). The particles are able to access the inside of the cells where they will release their contents. The subject matter of the present invention involves, in some cases, interrelated products, alternative solutions to a particular problem, and/or a plurality of different uses of one or more systems and/or articles.

The inventors have produced nanoentities, such as nanocapsules, comprising an inner portion surrounded by an outer shell, the outer shell comprising polysialic acid (PSA), the PSA bonded to targeting moieties, particularly the cell penetrating peptides Lyp-1 or cLyp-1. This can be seen in Example 1. They have also demonstrated that these nanocapsules are able to contain pharmaceutical agents, such as paclitaxel and docetaxel. Further, they show that said nanocapsules are more effective than the pharmaceutical agent alone in an orthotopic lung tumor model, due to the enhanced delivery of the agent into the tumour tissue (see Example 2). The inventors have also demonstrated that other targeting moeities can be used, e.g. CendR (see Example 3). Example 5 illustrates the formulation of PSA nanocapsules associated with paclitaxel and other anticancer drugs. The polymer, such as PSA and hyaluronic acid, can be linked to a hydrophobic moiety e.g. a C₁₆ alkyl group, as shown in Examples 6, 7 and 13.

The inventors have also successfully produced nanocapsules associated with a pharmaceutical agent which is a monoclonal antibody, as can be seen in Example 8 to 10 wherein different polymers and nanocapsules are used: PSA, PSA with tLyp-1, PSA functionalized with C₁₂ alkyl group, hyaluronic acid functionalized with C₁₆ and tLyp, polyglutamic acid (PGA), PGA/PEG, and polyaspartic acid/PEG. The antibodies tested are IgG2 and bevazimumab. The nanocapsules have been characterized in relation e.g. to their toxicity, stability and loading capacity (see Examples 10 and 11). Further, the produced nanocapsules were shown to interact with cells and further elicit the cell internalization of the associated antibody i.e. the nanocapsules were engulfed by the cell membrane and drawn into the cell where the antibodies were released (see Example 12).

Thus, in one aspect, the invention relates to a composition comprising a plurality of nanoentities comprising an inner portion surrounded by an outer shell, the outer shell comprising a polymer and a targeting moiety, the inner portion comprising at least one hydrophobic compound.

In another aspect, the invention relates to a composition comprising a plurality of nanoentities comprising an inner portion surrounded by an outer shell, the outer shell comprising a polymer, the inner portion comprising at least one hydrophobic compound, with the proviso that the at least about 90% of the polymer is not hyaluronic acid.

In another aspect, the invention is directed to the compositions comprising a plurality of manoentities, for use as medicaments.

In one aspect, the present invention is generally directed to a composition. According to one set of embodiments, the composition comprises a plurality of nanoentities, for example, nanocapsules, comprising an inner portion (or core) surrounded by an outer shell. In some cases, the outer shell comprises polymers such as PSA. The inner portion comprises at least one hydrophobic compound.

In some embodiments, the outer shell comprises a targeting moiety, that is, a molecule which allows the targeting or selective targeting of the nanostructure. In certain embodiments, the outer shell comprises a cell- and/or tumor/tissue-penetrating peptide. In some cases, the targeting moiety, and/or the cell penetrating peptide and/or the tumor/tissue penetrating peptide is chemically linked to the PSA.

The composition, in another set of embodiments, includes a plurality of nanocapsules comprising an inner portion surrounded by an outer shell. In some embodiments, the outer shell comprises PSA and a targeting moiety chemically linked to the PSA. In some cases, the targeting moiety comprises a peptide having a sequence Z¹X¹X²Z², wherein Z¹ is R or K, Z² is R or K, and X¹ and X² are each an amino acid residue. In some cases, the peptide comprises a sequence RGD, or a sequence NGR. For instance, the peptide comprises a sequence J¹RGD, J¹RGDJ², RGDJ², J¹NGR, J¹NGRJ², NGRJ², etc. (These K, R, N, G, D, etc. abbreviations are the standard one-letter codes for amino acid residues as used by those of ordinary skill in the art; see below for details). In some cases, the targeting moiety comprises a peptide having both Z¹X¹X²Z² and RGD sequences (e.g. an iRGD peptide) or Z¹X¹X²Z² and NGR sequences (e.g. an iNGR).

In another set of embodiments of another aspect, the composition comprises a plurality of nanoentities comprising an inner portion surrounded by an outer shell, the outer shell comprising a polymer such as PSA, at least some of the nanoentities further comprising a monoclonal antibody contained within the inner portion.

In another aspect, the composition comprises a plurality of nanocapsules comprising an inner portion surrounded by an outer shell, the outer shell comprising PSA and a targeting moiety chemically linked to the PSA, wherein the targeting moiety comprises a peptide having a sequence Z¹X¹X²Z² and/or a sequence RGD and/or a sequence NGR, wherein Z1 is R or K, Z² is R or K, and X¹ and X² are each an amino acid residue.

In accordance with yet another set of embodiments of another aspect, the composition comprises entities, having a maximum average diameter of less than about 1 micrometer. The entities, in some embodiments, have a surface comprising a polymer such as PSA and a targeting moiety. In some cases, the entities are not liposomes (See below for a discussion of liposomes).

Still another set of embodiments is generally directed to a composition comprising a plurality of nanoentities, for example, nanocapsules, comprising an inner portion surrounded by an outer shell. The outer shell comprises a polymer such as PSA, optionally linked to a hydrophobic moiety, e.g., covalently, electrostatically, etc. The inner portion comprises at least one hydrophobic compound in certain instances, n some embodiments, the outer shell comprises a polymer such as PSA, a targeting moiety and a hydrophobic moiety. In some cases, at least some of the PSA is linked to the targeting moiety and/or to the hydrophobic moiety. In some embodiments, the hydrophobic moiety is an alkyl group, such as C₂-C₂₄, or C₁₂.

In another aspect, the composition comprises a plurality of nanoentities comprising an inner portion surrounded by an outer shell, the outer shell comprising PSA and a targeting moiety comprising a cell-penetrating peptide chemically linked to the PSA.

In another set of embodiments of another aspect, the composition comprises a plurality of nanoentities, for example, nanocapsules, comprising an inner portion surrounded by an outer shell. In some cases, the outer shell consists essentially of a polymer such as PSA. In certain instances, the inner portion comprises at least one hydrophobic compound.

According to one aspect, the composition comprises a plurality of nanoentities comprising an inner portion surrounded by an outer shell, the outer shell comprising hyaluronic acid, at least some of the nanoentities further comprising a monoclonal antibody.

In another aspect, the composition comprises a plurality of nanoentities comprising an inner portion surrounded by an outer shell, the outer shell comprising PGA and/or PASP and a targeting moiety.

The composition, in yet another aspect, comprises: a plurality of nanocapsules comprising an inner portion surrounded by an outer shell, the outer shell comprising PGA and/or PASP and a targeting moiety, wherein the targeting moiety comprises a peptide having a sequence Z¹X¹X²Z² and/or a sequence RGD and/or a sequence NGR, wherein Z¹ is R or K, Z² is R or K, and X¹ and X² are each an amino acid residue.

In still another aspect, the composition comprises a plurality of nanoentities comprising an inner portion surrounded by an outer shell, the outer shell comprising PGA and/or PASP, at least some of the nanoentities further comprising a monoclonal antibody contained within the inner portion.

According to one aspect, the composition comprises a plurality of nanoentities comprising an inner portion surrounded by an outer shell, the outer shell comprising hyaluronic acid linked to a hydrophobic moiety

The composition, in another aspect, comprises a plurality of nanoentities comprising an inner portion surrounded by an outer shell, the outer shell comprising a polymer selected from the group consisting of polyacids, polyesters, polyamides, or mixtures thereof, at least some of the nanoentities further containing a monoclonal antibody.

The composition, in still another aspect, comprises a plurality of nanoentities comprising an inner portion surrounded by an outer shell, the outer shell comprising hyaluronic acid linked to a hydrophobic moiety, at least some of the nanoentities further comprising a small molecule have a molecular weight of less than 1000 Da.

In another set of embodiments, the composition is a pharmaceutical composition.

Additional embodiments of the invention are generally directed to the use of any of the above-described compositions, or any composition described herein, as a medicament. In addition, some embodiments of the invention are generally directed to a method of administering the composition of any of the above-described compositions, or any composition herein, to a living organism, such as a human. In some cases, the living organism is one subject with cancer, or other diseases. For instance, any of the above-described compositions (or any composition described herein) may further include a suitable therapeutic, such as an anticancer drug or an antibody.

Another aspect of the invention is generally directed to a method. In some embodiments, the method includes acts of reacting a carboxylate moiety on a PSA with an aminoalkyl (C₁-C₄) maleimide and/or with an aminoalkyl (C₁-C₄) methacrylamide, and reacting the resulting aminoalkyl (C₁-C₄) maleimide and/or the aminoalkyl (C₁-C₄) methacrylamide to a thiol group (for example from a cysteine group) on a targeting moiety to produce a PSA-aminoalkyl (C₁-C₄) succinimide-peptide and/or a PSA-aminoalkyl (C₁-C₄) amido-isopropyl-peptide composition. In some embodiments, the method includes acts of reacting a carboxylate moiety on a PSA with an activator, as for example a N-hydroxysuccinimide, a triazine or a carbodiimide, and reacting the intermediate formed with an amino group (for example from a lysine or arginine group) on a targeting moiety to produce a PSA-amide-peptide.

Several methods are disclosed herein of administering a subject with a compound for prevention or treatment of a particular condition. It is to be understood that in each such aspect of the invention, the invention specifically includes, also, the compound for use in the treatment or prevention of that particular condition, as well as use of the compound for the manufacture of a medicament for the treatment or prevention of that particular condition.

In another aspect, the present invention encompasses methods of making one or more of the embodiments described herein, for example, a nanocapsule. In still another aspect, the present invention encompasses methods of using one or more of the embodiments described herein, for example, a nanocapsule.

Other advantages and novel features of the present invention will become apparent from the following detailed description of various non-limiting embodiments of the invention when considered in conjunction with the accompanying figures.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIG. 1 illustrates a coupling reaction of sialic acid to a peptide intended to act as a targeting moiety;

FIGS. 2A-2B illustrate data showing delivery of nanocapsules to mice, in accordance with certain embodiments of the invention;

FIG. 3 illustrates a comparison of delivery of certain nanocapsules as described herein to Abraxane® (nab-paclitaxel);

FIG. 4 illustrates the evolution of body weight of mice treated with certain nanocapsules in yet another embodiment of the invention;

FIGS. 5A-5B illustrates in vivo efficacy of certain nanocapsules, in accordance with another embodiment of the invention;

FIG. 6 illustrates a method of producing a modified PSA, in accordance with another embodiment of the invention;

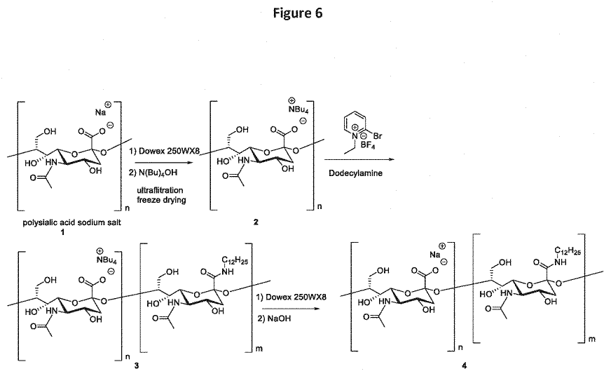

FIG. 7 illustrates the cytotoxicity of different polymeric nanocapsules, in yet other embodiments of the invention;

FIGS. 8A-8D illustrate the efficacy of delivery of polymeric nanocapsules to cells, in accordance with one embodiment of the invention.

FIGS. 9A-9B illustrates the stability of different mAb-loaded polymeric nanocapsules measured by DLS, in another embodiment of the invention;

FIGS. 10A-10C illustrate the stability of different mAb-loaded polymeric nanocapsules measured by NTA, in yet another embodiment of the invention;

FIG. 11 illustrates positive cells incubated with different polymeric nanocapsules, in still another embodiment of the invention;

FIGS. 12A-12B illustrate cells loaded with nanocapsules, in yet another embodiment of the invention; and

FIGS. 13A-13C illustrate ¹H-NMR spectra for PSA, tLyp1 and the conjugate PSA-tLyp1, in certain embodiments of the invention.

### DETAILED DESCRIPTION OF THE INVENTION

The present invention generally relates to particles, including nanocapsules or other nanoentities, comprising a polymer such as polysialic acid (PSA). The particles are able to access the interior of the cells, and/or to procure the intracellular release of the associated drugs. In one aspect, the present invention is directed to nanocapsules or other entities having an exterior or surface comprising a polymer such as PSA. In some cases, targeting moieties such as Lyp-1 or tLyp-1 peptide are bonded to the polymer, e.g., using aminoalkyl (C₁-C₄) succinimide or other linkers. These are created, for example, by reacting a carboxylate moiety on a polymer with an aminoalkyl maleimide (C₁-C₄) or an aminoalkyl (C₁-C₄) methacrylamide and reacting the resulting aminoalkyl (C₁-C₄) maleimide or the aminoalkyl (C₁-C₄) methacrylamide to a cysteine or other sulfur group. Targeting moieties are bonded to the polymer, for example, by reacting a carboxylate moiety on a polymer with a N-hydroxysuccinimide or a carbodiimide, and reacting the intermediate formed with a lysine or arginine group on a targeting peptide to produce polymer-amide-peptide. Other aspects of the invention are generally directed to methods of making or using such compositions, kits including such compositions, or the like.

Applications of the Entities

In one aspect, the present invention is generally directed to particles or other entities comprising polymers such as PSA. Such particles or entities are used, for example, for drug delivery applications. For example, such particles are delivered into a subject such that they reach a tumor that the subject is suffering from. The particles are delivered into the tumor cells, for example, facilitated by a targeting moiety which also have capacity as cell- or tissue-penetrating peptides such as Lyp-1 or tLyp-1, or other peptides discussed herein (e.g., CendR peptides). Other peptides, antibodies (e.g. full-length antibodies, nanobodies, single chain variable fragments, etc.), or aptamer targeting moieties, are also used in certain embodiments, e.g., as discussed herein. Once delivered, the particles can access the target cells, for example tumor cells, and release the drug contained therein (e.g., therapeutic or anticancer drugs, etc.). Particles or other entities comprising modified PSA with a targeting moiety have not previously been used for the selective and intracellular release of drugs.

In some cases, the entities are present within a pharmaceutically acceptable carrier, as discussed herein; for instance, the entities are suspended in a liquid or a gel, e.g., for administration to a subject. The entities are substantially solid, or may define internal spaces, e.g., as in a capsule. The entities are also a micelle or a liposome in some embodiments, although in certain cases, the entities as discussed herein are not liposomes.

Entities—Nanoentities

“Entity” includes for example, capsules, particles, and micelles. In some cases, the entity is a nanoentity. A “nanoentity,” as used herein, typically is an entity that has an average diameter of less than 1,000 nm, e.g., less than 750 nm, less than 500 nm, less than 300 nm, less than 250 nm, less than 200 nm, less than 150 nm, or less than 100 nm. In some cases, the entities have an average diameter of at least 1 nm, 5 nm, 10 nm, 50 nm, 100 nm, 500 nm, or 1,000 nm. Combinations of any of these diameters are also possible, for instance, the entity has an average range of diameters of between 100 nm and 300 nm between 1,000 nm and 1 nm, between 1,000 nm and 10 nm, between 750 nm and 1 nm, between 500 nm and 10 nm, between 300 nm and 10 nm, between 250 nm and 10 nm, between 200 nm and 10 nm, between 150 nm and 10 nm, between 100 nm and 10 nm, or the like. More than one entity are also present in some embodiments, and in such cases, the average (arithmetic) diameter of the plurality of entities have the dimensions described here. In some cases, entities having a range of diameters are present. Such entities are determined by a variety of methods, such as dynamic or laser light scattering techniques. Non-limiting examples of nanoentities include nanoparticles, nanocapsules, micelles, or other entities such as those described herein. Such nanoentities have, in some cases, the dimensions provided in this paragraph.

In some cases, the entity includes an inner portion surrounded by an outer shell, e.g., exposed to the environment surrounding the entity. The inner portion is symmetrically or asymmetrically positioned within the entity. The inner portion contains, for example, a liquid (which is, e.g., nonaqueous or aqueous), a solid and/or combinations thereof. In some embodiments, the inner portion contains one or more pharmaceutical agents or drugs, for example, any of those described herein. For example, the inner portion contains a monoclonal antibody, or a small molecule such as docetaxel. In some cases, the inner portion (including the contained moiety) is prevented from being exposed to the external environment, e.g., due to the outer shell.

Entities—Capsules/Nanocapsules, Particles/Nanoparticles

In some cases, the entity is a capsule (e.g., a nanocapsule). The capsule is substantially solid, or have a rubbery or gel-like shell. In addition, in some cases, the entity is a particle, such as a nanoparticle. The particle is solid and have a well-defined shape. In some cases, the particle is an entity having an inner portion surrounded by an outer shell, e.g., the particle is a capsule. The nanocapsule has a size in the nanometer range. If the nanoparticle is generally spherical, it can also be referred to nanosphere. A nanocapsule is substantially uniform, although it has additional surface features, such as targeting moieties, penetration enhancers, antibodies, or the like, including those described herein.

In some cases, the particle is an entity having an inner portion surrounded by an outer shell, e.g., the particle is a capsule or a nanocapsule. In some cases, a nanocapsule has a size in the nanometer range comprising an inner core and an outer shell having a composition distinguishable from the inner core. The inner core can be, e.g., a liquid or a solid material. Often but not always, the inner core is an oil. The outer shell is formed from a continuous material, and is typically not covalently attached to the inner core. In some cases, the outer shell has an average thickness of at least 1 nm, at least 2 nm, at least 3 nm, at least 5 nm, at least 10 nm, at least 20 nm, at least 30 nm, at least 50 nm, at least 100 nm, or at least 200 nm.

In some cases, the nanoentity comprises no more than one outer shell.

Entities—Micelles

In some cases, the entity is a micelle. Typically, a micelle is formed from a plurality of surfactant or amphiphilic molecules that defines an inner portion and an exterior. For example, the surfactant molecules are arranged to have a relatively hydrophilic exterior and a relatively hydrophobic inner portion, e.g., formed from a single layer of surfactant or amphiphilic molecules. In some cases, the micelle has a size in the nanometer range. The micelle is, in some embodiments, composed by amphiphilic molecules at a concentration above the CMC (critical micellar concentration) when the micelles are dispersed in an external phase. If the external liquid phase is aqueous, the hydrophilic part of the amphiphilic molecules is oriented towards the external phase. Depending on the concentration of amphiphilic molecules, the micelles can organize themselves forming larger structures, which are clusters of micelles. Micelles are formed from surfactant molecules, e.g., having their hydrophilic portions on the surface and their hydrophobic portions pointing inwardly (or vice versa in some cases).

Entities—Liposomes

A liposome can have a similar structure, but is usually formed from a double layer of surfactant or amphiphilic molecules (e.g., a lipid bilayer), and may thereby define an inner portion, a middle portion, and an outer shell; for example, the inner portion is relatively hydrophilic, the middle portion (e.g., the outer shell of the liposome, formed by the bilayer structure of the surfactant or amphiphilic molecules) is relatively hydrophobic, and the exterior to the liposome is an aqueous or a hydrophilic environment.

As used herein, the property of being “hydrophilic” is understood as the constitutional property of a molecule or functional group to penetrate into the aqueous phase or to remain therein. Accordingly, the property of being “hydrophobic” is understood as a constitutional property of a molecule or functional group to exhibit exophilic behavior with respect to water; i.e., they display the tendency to not penetrate into water, or to depart the aqueous phase. For further details reference is made to Rompp Lexikon Lacke und Druckfarben, Georg Thieme Verlag, Stuttgart, N.Y., 1998, “Hydrophilicity”, “Hydrophobicity”, pages 294 and 295. In some cases, a hydrophilic (or hydrosoluble) entity is one that exhibits a log P of less than 1.5, while a hydrophobic (or liposoluble) entity is one that exhibits a log P of greater than 1.5, where log P is the octanol-water partition coefficient of the entity.

The inner portion, if present within an entity, contains a liquid, and in some cases, the liquid is aqueous or nonaqueous. In some cases, the liquid contains saline or a salt solution in water. Optionally, the liquid can contain a drug or other pharmaceutical agent, e.g., for delivery to a subject. Non-limiting examples of drugs or other pharmaceutical agents are discussed herein. For example, the inner portion contains a monoclonal antibody, or a small molecule such as docetaxel.

In some embodiments, the nanoentity comprises an outer shell consisting essentially of single layer of material comprising a polymer, such as PSA. In other embodiments, the nanoentity comprises a single shell comprising a polymer, such as PSA. In other embodiments, the outer shell comprises multiple layers, wherein one of the layers comprises a polymer, such as PSA. In further embodiments, the layer comprising the polymer is the outermost layer.

In some embodiments, the inner portion of the nanoentity, e.g., nanocapsule, nanoparticle, micelle, or liposome, comprises a solid, semi-solid (e.g., gel), liquid, gas, or combination thereof. The inner portion is aqueous, non-aqueous, or comprise both an aqueous and non-aqueous portion. In some embodiments, the inner portion comprises one or more pharmaceutical agents, drugs, or the like.

In other embodiments, the inner portion comprises a non-aqueous portion. In further embodiments, the non-aqueous portion is a non-aqueous liquid. In further embodiments, the non-aqueous liquid comprises a hydrophobic compound, e.g., an oil. In further embodiments, the non-aqueous liquid comprises an oil and a surfactant. In further embodiments, the inner portion comprises a fatty acid. In further embodiments, the inner portion comprises a monoglyceride. In further embodiments, the inner portion comprises a diglyceride. In further embodiments, the inner portion comprises a triglyceride. In further embodiments, the inner portion comprises a medium chain triglyceride. In further embodiments, the inner portion comprises a long chain triglyceride.

Hydrophobic Compounds

If the inner portion of an entity (for example, capsules, particles, micelles, or other nanoentities such as those discussed herein) is nonaqueous, the nonaqueous liquid forming the inner portion comprises one or more hydrophobic compounds, for example, selected from oil, fatty acid, alkane, cycloalkane, bile salt, bile salt derivatives, terpenoid, terpene, terpene-derived moieties and lipophilic vitamin, and/or at least one surfactant. These oils can be selected from natural, semi-synthetic and synthetic oils for pharmaceutical use, such as oils from a plant or animal origin, hydrocarbon oils or silicone oils. Oils suitable for carrying out certain embodiments of the present invention include, but are not limited to, mineral oil, squalene oil, flavored oils, silicone oil, essential oils, water-insoluble vitamins, isopropyl stearate, butyl stearate, octyl palmitate, cetyl palmitate, tridecyl behenate, diisopropyl adipate, dioctyl sebacate, menthyl anthranilate, cetyl octanoate, octyl salicylate, isopropyl myristate, neopentyl glycol dicaprate ketols, decyl oleate, C₁₂-C₁₅ alkyl lactates, cetyl lactate, lauryl lactate, isostearyl neopentanoate, myristyl lactate, isocetyl stearoyl stearate, octyldodecyl stearoyl stearate, hydrocarbon oils, isoparaffin, fluid paraffins, isododecane, petroleum jelly, argan oil, rapeseed oil, chili oil, coconut oil, corn oil, cottonseed oil, linseed oil, grape seed oil, mustard oil, olive oil, palm oil, fractionated palm oil, peanut oil, castor oil, pine nut oil, poppy seed oil, pumpkin seed oil, rice bran oil, safflower oil, tea tree oil, truffle oil, vegetable oil, apricot kernel oil, jojoba oil, macadamia nut oil, wheat germ oil, almond oil, soybean oil, sesame seed oil, hazelnut oil, sunflower oil, hempseed oil, rosewood oil, Kukui nut oil, avocado oil, walnut oil, fish oil, berry oil, allspice oil, juniper oil, seed oil, almond seed oil, anise seed oil, celery seed oil, cumin seed oil, nutmeg seed oil, basil leaf oil, bay leaf oil, cinnamon leaf oil, common sage leaf oil, eucalyptus leaf oil, lemon leaf oil, melaleuca leaf oil, oregano oil, patchouli leaf oil, peppermint leaf oil, pine needle oil, rosemary leaf oil, spearmint oil, tea tree leaf oil, thyme oil, flower oil, chamomile oil, clary sage oil, clove oil, geranium flower oil, hyssop flower oil, jasmine oil, lavender oil, mauka flower oil, marjoram flower oil, orange flower oil, rose flower oil, ylang-ylang flower oil, bark oil, cassia bark oil, cinnamon bark oil, sassafras bark oil, wood oil, camphor wood oil, cedarwood oil, rosewood oil, sandalwood oil, ginger wood oil, tall oil, castor oil, myrrh oil, peel oil, Bergamot peel oil, grapefruit peel oil, lemon peel oil, lime peel oil, orange peel oil, tangerine peel oil, root oil, valerian oil, oleic acid, linoleic acid, oleyl alcohol, isostearyl alcohol, ethyl oleate, medium-chain triglycerides such as mixtures of decanoyl- and octanoyl glycerides (Miglyol® 8 ION, Miglyol® 812N, Kollisolv® MCT, Captex® 300, Captex® 355, Labrafac® Lipophile WL1349), Labrafil® M 2125 CS (Linoleoyl macrogol-6 glycerides), Labrafil® M2130 CS (Lauroyl macrogol-6 glycerides), Labrafil® M 1944 CS (oleoyl polyoxyl-6 glycerides), Labrafac® PG (propylene glycol dicaprylocaprate), Rylo® (mixture of fatty acids), Peceol® (glycerol monooleate) and Maisine® (glycerol monolinoleate), synthetic or semi-synthetic derivatives thereof and combinations thereof.

In some cases, the oil is one or more of peanut oil, cottonseed oil, olive oil, castor oil, soybean oil, safflower oil, sesame oil, corn oil, palm oil, alpha-tocopherol (vitamin E), isopropyl myristate, squalene, Miglyol®, Labrafil®, Labrafac®, Peceol®, Captex®, Kollisolv® MCT and Maisine® or mixtures thereof. Other suitable oils include oils from the terpene family formed by isoprene units (2-methylbuta-1,3-diene) and sub-divided according to their carbon atoms: hemiterpenes (C₅), monoterpenes (C₁₀), sesquiterpenes (C₁₅), diterpenes (C₂₀), sesterterpenes (C₂₅), triterpenes (C₃₀), tetraterpenes (C₄₀, carotenoids) and polyterpenes, vitamin A, squalene, etc. In some embodiments, the nonaqueous liquid forming the inner portion can contain water-insoluble stabilizers, preservatives, surfactants, organic solvents and mixtures thereof to provide maximum stability of the formulation. Combinations of one or more of these and/or other oils are also possible in various embodiments.

If the inner portion of an entity (for example, capsules, particles, micelles, or other nanoentities such as those discussed herein) is aqueous, the aqueous liquid forming the inner portion can be made up of water containing at least one salt, in certain embodiments.

Additionally, in some embodiments, the aqueous liquid forming the inner portion can contain one or more water-soluble stabilizers, preservatives, surfactants, glycols, polyols, sugars, thickening agents, gelling agents, and mixtures of these and/or other suitable excipients. These excipients are used, for instance, to improve stability of the formulation, adjust the viscosity of the final composition, control the rate of release from the inner aqueous phase, or the like.

Polymers

In one set of embodiments, the entities (e.g., capsules, particles, micelles, or other nanoentities such as those discussed herein) comprise a polymer, such as PSA. The polymer is evenly distributed throughout the entity, or concentrated within certain regions of the entity, e.g., in the outer shell of a capsule, or other outer surface of an entity. In some cases, at least 50 wt % of a portion of an entity, such as a shell, comprises the polymer, and in certain cases, at least 60 wt %, at least 70 wt %, at least 75 wt %, at least 80 wt %, at least 85 wt %, at least 90 wt %, at least 95 wt %, or at least 99 wt % of the portion of the entity can comprise the polymer. In some cases, a portion of the entity can consist essentially of the polymer.

A variety of polymers are used in accordance to certain embodiments of the invention. For example, the polymer is a polyacid, poly(amino acid) or a polyester in one set of embodiments. Non-limiting examples of these polymers include PSA, hyaluronic acid (HA), polyglutamic acid (PGA), pegylated polyglutamic (PGA-PEG), poly(aspartic acid) (PASP), pegylated polyaspartic (PASP-PEG), polylactic acid, pegylated polylactic (PLA-PEG), pegylated poly (lactic-co-glycolic acid) (PLGA-PEG), polyasparaginic acid, pegylated polyasparaginic acid, alginic acid, pegylated alginic acid, polymalic acid, pegylated polymalic acid, or the like. Combinations of these and/or other polymers are also used in certain embodiments. For example, such polymers are used to form a nanoentity containing a monoclonal antibody or a small molecule, e.g., contained within an inner portion of the nanoentity, or other applications such as those described herein.

Polymers—Polysialic Acid, PSA

According to one set of embodiments, the polymer comprises PSA. PSA is generally composed of a plurality of sialic acid units, often bonded together to form a polymer via 2->8 and/or 2->9 bonding, although other bonding arrangements are also possible. Typically, there are at least 2, at least 4, at least 6, at least 8, at least 10, at least 15, at least 20, at least 25, at least 30, at least 40, at least 50, at least 75, at least 100, at least 200, at least 300, at least 400, or at least 500 sialic acid units bonded together to form PSA. In some cases, the PSA has no more than 1000, no more than 500, no more than 200, no more than 100, no more than 50, no more than 30, or no more than 10 sialic acid units bonded together to form the PSA. Combinations of any of these are also possible, e.g., a PSA has between 2 and 100 sialic acid units that are bonded together. It should be noted that the sialic acid units need not be identical, and can independently be the same or different, even within the same PSA molecule. It should also be noted that a PSA need not necessarily be a straight (linear) chain, and various branching arrangements are also possible. For instance, a sialic acid unit is bonded to 3 or more different sialic acid units, thereby creating a branch point within the PSA molecule.

As non-limiting examples, the PSA has different molecular weights, e.g., 4 kDa, 30 kDa, 95 kDa, etc. In some cases, the PSA contains more than 300 sialic acid units. As additional non-limiting examples, the PSA has a molecular weight of at least 1 kDa, at least 3 kDa, at least 5 kDa, at least 10 kDa, at least 20 kDa, at least 25 kDa, at least 30 kDa, at least 40 kDa, at least 50 kDa, at least 60 kDa, at least 70 kDa, at least 75 kDa, at least 80 kDa, at least 90 kDa, at least 100 kDa etc. In some cases, the PSA has a molecular weight of no more than 100 kDa, no more than 90 kDa, no more than 80 kDa, no more than 75 kDa, no more than 70 kDa, no more than 60 kDa, no more than 50 kDa, no more than 40 kDa, no more than 30 kDa, no more than 25 kDa, no more than 20 kDa, no more than 10 kDa, no more than 5 kDa, no more than 3 kDa, or no more than 1 kDa. Combinations of any these are also possible, e.g., the PSA has a molecular weight between about 1 kDa and about 100 kDa, between about 5 kDa and about 80 kDa, or between about 10 kDa and about 50 kDa, etc. (Unless indicated to the contrary, molecular weights described herein are number average molecular weights).

It should also be noted that the polysialic acids need not always be identical. For example, in some embodiments, the PSAs have different numbers of sialic acid units, and/or there are different sialic acid units in different PSA molecules that are present. In some cases, one or a few types of PSA molecules may be present, e.g., one or more forms comprise at least 30%, at least 40%, at least 50%, at least 60%, at least 70%, at least 80%, at least 90%, or more of the PSA molecules that are present, i.e., on a molar basis.

Non-limiting examples of sialic acid units that are present within a PSA include, but are not limited to, N-acetylneuraminic acid (Neu), 2-keto-3-deoxynonic acid (Kdn), lactaminic acid, N-sialic acid, and/or O-sialic acid. Other examples include N-glycolylneuraminic (Neu5Gc), 9-O-acetyl-8-O-methyl-A-acetylneuraminic acid (Neu5,9Ac28Me), and 7,8,9-tri-O-acetyl-N-glycolyl neuraminic acid (Neu5Gc7,8,9Ac3). “Sia” generally denotes an unspecified sialic acid unit. In some embodiments, the sialic acid units include any derivative of neuraminic acid (a 9-carbon sugar), including the 43 derivatives typically found in nature. These include, but are not limited to, Neu; Neu5Ac; Neu4,5Ac₂; Neu5,7Ac₂; Neu5,8Ac₂; Neu5,9Ac₂; Neu4,5,9Ac₃; Neu5,7,9Ac₃; Neu5,8,9Ac₃; Neu5,7,8,9Ac₄; Neu5Ac9Lt; Neu4,5Ac₂9Lt; Neu5Ac8Me; Neu5,9Ac₂8Me; Neu5Ac8S; Neu5Ac9P; Neu2en5Ac; Neu2en5,9Ac₂; Neu2en5Ac9Lt; Neu2,7an5Ac; Neu5Gc; Neu4Ac5Gc; Neu7Ac5Gc; Neu8Ac5Gc; Neu9Ac5Gc; Neu7,9Ac₂5Gc; Neu8,9Ac₂5Gc; Neu7,8,9Ac₃5Gc; Neu5Gc9Lt; Neu5Gc8Me; Neu9Ac5Gc8Me; Neu7,9Ac₂5Gc8Me; Neu5Gc8S; Neu5GcAc; Neu5GcMe; Neu2en5Gc; Neu2en9Ac5Gc; Neu2en5Gc9Lt; Neu2en5Gc8Me; Neu2,7an5Gc; Neu2,7an5Gc8Me; Kdn; and Knd9Ac. In one set of embodiments, each of the sialic acid units (prior to polymerization to form PSA) can independently have the following structure:

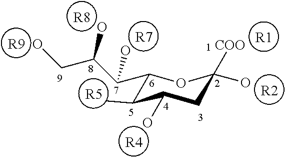

R¹ is H; an alpha linkage to Gal(3/4/6), GalNAc(6) (A-acetylgalactosamine), GlcNAc(4/6), Sia (8/9), or 5-O-Neu5Gc; an oxygen linked to C-7 in 2,7-anhydro molecule; or an anomeric hydroxyl eliminated in Neu2en5Ac (double bond to C-3). R² is H; an alpha linkage to Gal(3/4/6), GalNAc(6), GlcNAc(4/6), Sia (8/9), or 5-O-Neu5Gc; an oxygen linked to C-7 in 2,7-anhydro molecule; or an anomeric hydroxyl eliminated in Neu2en5Ac (double bond to C-3). R⁴ is H; -acetyl; an anhydro to C-8; Fuc (fucose); or Gal (galactose). R⁵ is an amino; N-acetyl; N-glycolyl; hydroxyl; N-acetimidoyl; N-glycolyl-O-acetyl; N-glycolyl-O-methyl; or N-glycolyl-O-2-Neu5Gc. R⁷ is H; -acetyl; an anhydro to C-2; or substituted by amino and N-acetyl in Leg (legionaminic acid). R⁸ is H; -acetyl; an anhydro to C-4; -methyl; -sulfate; Sia (sialic acid); or Glc (glucose). R⁹ is H; -acetyl; -lactyl; -phosphate; -sulfate; Sia; or OH substituted by H in Leg. In some cases, the PSA is colominic acid (where only 2->8 bonding is present).

As used herein, sialic acid includes water-soluble salts and water-soluble derivatives of sialic acid. For example, the sialic acid salt is the sodium salt, the potassium salt, the magnesium salt, the calcium salt, or the zinc salt. In one embodiment, at least some of the sialic acid is present as a sodium salt. Combinations of multiple types of sialic acids are also used, e.g., as subunits of a PSA, and/or as different molecules of PSA.

In one set of embodiments, at least some of the sialic acid within PSA is modified (however, it should be understood that in other embodiments, the PSA is not necessarily modified). For instance, in some cases, one or more sialic acid units are modified, for example, by attachment to polyethylene glycol, alkyl or other hydrophobic moieties, or the like. Hydrophobic moieties include hydrophobic molecules or portions thereof, e.g., an alkyl group, such as those discussed herein.

In some embodiments, the nanoentities does not comprise polyarginine or protamine.

However, it should be understood that other polymers are also used, e.g., in addition and/or instead of PSA.

Polymers—Hyaluronic Acid, HA

In one set of embodiments, the polymer comprises hyaluronic acid. Hyaluronic acid is a linear polymer comprising the repetition of a disaccharide structure formed by the alternating addition of D-glucuronic acid and D-N-acetylglucosamine bound by alternating beta-1,4 and beta-1,3 glycosidic bonds as shown in the following formula:

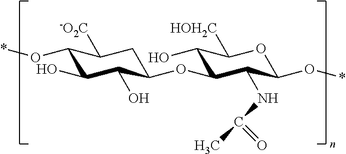

wherein the integer n represents the degree of polymerization, i.e., the number of disaccharide units in the hyaluronic acid chain. For example, n is at least 2, at least 4, at least 6, at least 8, at least 10, at least 15, at least 20, at least 25, at least 30, at least 40, at least 50, at least 75, at least 100, at least 200, at least 300, at least 400, or at least 500. In some cases, n is no more than 1000, no more than 500, no more than 200, no more than 100, no more than 50, no more than 30, or no more than 10. Combinations of any of these are also possible, e.g., n is between 2 and 100. It should be noted that the hyaluronic acid units need not be identical, and can independently be the same or different, even within the same hyaluronic acid chain. It should also be noted that hyaluronic acid need not necessarily be a straight (linear) chain, and various branching arrangements are also possible.

Thus, hyaluronic acid with a wide range of molecular weights can be used. Higher molecular weight hyaluronic acid is commercially available, whereas lower molecular weight hyaluronic acid can be obtained by means of fragmenting the hyaluronic high molecular weight acid using a hyaluronidase enzyme, for example. As non-limiting examples, the hyaluronic acid has different molecular weights, e.g., 4 kDa, 30 kDa, 95 kDa, etc. For example, hyaluronic acid has a molecular weight of at least 1 kDa, at least 3 kDa, at least 5 kDa, at least 10 kDa, at least 20 kDa, at least 25 kDa, at least 30 kDa, at least 40 kDa, at least 50 kDa, at least 60 kDa, at least 70 kDa, at least 75 kDa, at least 80 kDa, at least 90 kDa, at least 100 kDa etc. In some cases, hyaluronic acid has a molecular weight of no more than 100 kDa, no more than 90 kDa, no more than 80 kDa, no more than 75 kDa, no more than 70 kDa, no more than 60 kDa, no more than 50 kDa, no more than 40 kDa, no more than 30 kDa, no more than 25 kDa, no more than 20 kDa, no more than 10 kDa, no more than 5 kDa, no more than 3 kDa, or no more than 1 kDa. Combinations of any these are also possible, e.g., the hyaluronic acid has a molecular weight between about 1 kDa and about 100 kDa, between about 5 kDa and about 80 kDa, or between about 10 kDa and about 50 kDa, etc.

Hyaluronic acid, as used herein, also includes its conjugated base (hyaluronate). This conjugated base can be an alkaline salt of hyaluronic acid including inorganic salts such as, for example, sodium salt, potassium salt, calcium salt, ammonium salt, magnesium salt, aluminium salt and lithium salt, organic salts such as basic amino acid salts at neutral pH. In some cases, the salts are pharmaceutically acceptable. In one embodiment, the alkaline salt is the sodium salt of hyaluronic acid. Combinations of multiple types of hyaluronic acid are also used, e.g., as subunits of a hyaluronic acid chain, and/or as different molecules of hyaluronic acid.

Thus, the hyaluronic acids need not always be identical. For example, in some embodiments, the hyaluronic acids have different numbers of hyaluronic acid units (such as those described above), and/or there are different hyaluronic acid units in different hyaluronic acid chains that are present. In some cases, one or more types of hyaluronic acid molecules are present, e.g., one or more forms comprise at least 30%, at least 40%, at least 50%, at least 60%, at least 70%, at least 80%, at least 90%, or more of the hyaluronic acid molecules that are present, i.e., on a molar basis.

In some embodiments, at least some of the hyaluronic acid units are modified (however, it should be understood that in other embodiments, the hyaluronic acid is not necessarily modified). For instance, in some cases, one or more hyaluronic acid units are modified, for example, by attachment to polyethylene glycol, alkyl or other hydrophobic moieties, or the like. Hydrophobic moieties include hydrophobic molecules or portions thereof, e.g., an alkyl group, such as those discussed herein.

Polymers—Polyglutamic Acid, PGA

In another set of embodiments, the polymer comprises polyglutamic acid (PGA). Polyglutamic acid (PGA) is a hydrophilic and biodegradable polymer of glutamic units that are negatively charged. It can be represented by the following formula:

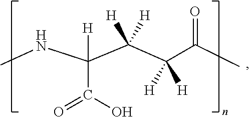

wherein the integer n represents the degree of polymerization, i.e., the number of glutamic units. For example, n is at least 2, at least 4, at least 6, at least 8, at least 10, at least 15, at least 20, at least 25, at least 30, at least 40, at least 50, at least 75, at least 100, at least 200, at least 300, at least 400, or at least 500. In some cases, n is no more than 1000, no more than 500, no more than 200, no more than 100, no more than 50, no more than 30, or no more than 10. Combinations of any of these are also possible, e.g., n is between 2 and 100. It should be noted that the hyaluronic glutamic units need not be identical, and can independently be the same or different, even within the same polyglutamic acid. Examples of such glutamic units include those discussed below. It should also be noted that glutamic units need not necessarily be a straight (linear) chain, and various branching arrangements are also possible.

Thus, polyglutamic acids with a wide range of molecular weights can be used. As non-limiting examples, the polyglutamic acid has different molecular weights, e.g., 4 kDa, 30 kDa, 95 kDa, etc. For example, the polyglutamic acid has a molecular weight of at least 1 kDa, at least 3 kDa, at least 5 kDa, at least 10 kDa, at least 20 kDa, at least 25 kDa, at least 30 kDa, at least 40 kDa, at least 50 kDa, at least 60 kDa, at least 70 kDa, at least 75 kDa, at least 80 kDa, at least 90 kDa, at least 100 kDa etc. In some cases, hyaluronic acid has a molecular weight of no more than 100 kDa, no more than 90 kDa, no more than 80 kDa, no more than 75 kDa, no more than 70 kDa, no more than 60 kDa, no more than 50 kDa, no more than 40 kDa, no more than 30 kDa, no more than 25 kDa, no more than 20 kDa, no more than 10 kDa, no more than 5 kDa, no more than 3 kDa, or no more than 1 kDa. Combinations of any these are also possible, e.g., the polyglutamic acid has a molecular weight between about 1 kDa and about 100 kDa, between about 5 kDa and about 80 kDa, or between about 10 kDa and about 50 kDa, etc.

As used herein, polyglutamic acids (or PGA) includes, but is not limited to, its conjugated base (glutamate), and/or water soluble salts of PGA, as the ammonium salt and metal salts of PGA, as the lithium salt, sodium salt, potassium salt, magnesium salt, etc. In one embodiment, PGA includes, for example, poly-D-glutamic acid, poly-L-glutamic L-glutamic acid, poly-D acid, poly-glutamic acid, poly-D-glutamic, glutamic poly- and poly-alpha-L-glutamic acid, poly-alpha-D acid, L-glutamic acid, poly-gamma-D-glutamic acid, poly-gamma-L-glutamic acid and poly-gamma-D, L-glutamic, and mixtures thereof. In another embodiment, PGA is present as poly-L-glutamic. In some cases, the PGA is present as the sodium salt of poly-L-glutamic acid. In another embodiment, the PGA is present as poly-alpha-glutamic acid. In still another embodiment, the PGA is present as the sodium salt of poly-a-glutamic acid. As mentioned, combinations of multiple types of polyglutamic acid are also used, e.g., as subunits of a polyglutamic acid chain, and/or as different molecules of polyglutamic acid.

Thus, the polyglutamic acids need not always be identical. For example, in some embodiments, the polyglutamic acids have different numbers of glutamate units (such as those described above), and/or there are different polyglutamic acids in different polyglutamic acid chains that are present. In some cases, one or more types of polyglutamic acid molecules are present, e.g., one or more forms comprise at least 30%, at least 40%, at least 50%, at least 60%, at least 70%, at least 80%, at least 90%, or more of the polyglutamic acids that are present, i.e., on a molar basis.

In some embodiments, at least some of the polyglutamic acid units are modified (however, it should be understood that in other embodiments, the polyglutamic acid is not necessarily modified). For instance, in some cases, one or more polyglutamic acid units are modified, for example, by attachment to polyethylene glycol, alkyl or other hydrophobic moieties, or the like. Hydrophobic moieties include hydrophobic molecules or portions thereof, e.g., an alkyl group, such as those discussed herein.

Polymers—Poly(Ethylene Glycol), PEG

In one set of embodiments, the polymer comprises poly(ethylene glycol) (PEG). In some cases, the PEG is conjugated to PGA, e.g., to form a polyglutamic-polyethyleneglycol acid copolymer (PGA-PEG). However, in other cases, PEG is present, i.e., not conjugated to PGA.

Polyethylene glycol (PEG), in its most common form, is a polymer having a formula:

H—(O—CH₂—CH₂)ₙ—OH,

where n is an integer representing the PEG polymerization degree. For the formation of the conjugate PGA-PEG, one or two of the two terminal hydroxyl groups are modified. The modified PEGs, e.g., as follows:

X¹—(O—CH₂—CH₂)ₙ—X²,

where X¹ is hydrogen or a hydroxyl protecting group blocking the OH radical function for subsequent reactions. For example, n is at least 2, at least 4, at least 6, at least 8, at least 10, at least 15, at least 20, at least 25, at least 30, at least 40, at least 50, at least 75, at least 100, at least 200, at least 300, at least 400, or at least 500. In some cases, n is no more than 1000, no more than 500, no more than 200, no more than 100, no more than 50, no more than 30, or no more than 10. Combinations of any of these are also possible, e.g., n is between 2 and 100.

The protecting groups of hydroxyl radicals are widely known in the art; Representative protecting groups (including oxygen) are, for example, silyl ethers such as trimethylsilyl ether, triethylsilyl ether, tert-butyldimethylsilyl ether, tert-butyldiphenylsilyl ether, triisopropylsilyl ether, diethylisopropylsilyl ether, triethyldimethylsilyl ether, triphenylsilyl ether, di-tert-butylmethylsilyl ether; alkyl ethers such as methyl ether, tert-butyl ether, benzyl ether, p-methoxybenzyl ether of 3,4-dimethoxybenzyl ether, triethyl ether, allyl ether; alkoxymethyl ethers such as methoxymethyl ether, 2-methoxyethoxymethyl, benzyloxymethyl ether, p-methoxybenzyloxymethyl ether, 2-(trimethylsilyl)ethoxymethyl ether; tetrahydropyranyl ethers and related ethers; methylthiomethyl ether; esters such as acetate ester, benzoate ester, pivalate ester, methoxyacetate ester, chloroacetate ester, levulinate ester; carbonates such as benzyl carbonate, p-nitrobenzyl carbonate, tert-butyl carbonate, 2,2,2-trichloroethyl carbonate, 2-(trimethylsilyl) ethyl allyl carbonate. As specific examples, the protecting group is an alkyl ether, such as methyl ether. X² is a bridge group allowing the anchoring to polyglutamic acid groups and groups derived therefrom. In some cases, X² can be a group allowing the anchoring with other PGA and derivatives thereof.

Polymers—PGA/PEG

In some cases, the PEGs are attached to PGA and their derivatives via amine groups and/or carboxylic acid of the latter. Pegylation of the polymers can be performed using any suitable method available in the art.

Such polymers are available in a variety of molecular weights. For example, a suitable molecular weight for PEG or PGA-PEG is between about 1 kDa and about 100 kDa, between about 5 kDa and about 80 kDa, between about 10 kDa and about 50 kDa, or about 10 kDa, about 15 kDa, about 20 kDa, about 25 kDa, about 30 kDa, and about 35 kDa.

A another example, a suitable molecular weight for PEG or PGA-PEG and water soluble derivatives thereof can be between about 1 kDa and about 50 kDa, between about 2 kDa and about 40 kDa, between about 3 kDa and about 30 kDa, or about 4 kDa, about 5 kDa, about 6 kDa, about 7 kDa, about 8 kDa, about 10 kDa, about 15 kDa, about 20 kDa, about 21 kDa, about 22 kDa, about 23 kDa, about 24 kDa, about 25 kDa, or about 30 kDa.

As additional non-limiting examples, the PEG or PGA-PEG has a molecular weight of at least 1 kDa, at least 3 kDa, at least 5 kDa, at least 10 kDa, at least 20 kDa, at least 25 kDa, at least 30 kDa, at least 40 kDa, at least 50 kDa, at least 60 kDa, at least 70 kDa, at least 75 kDa, at least 80 kDa, at least 90 kDa, at least 100 kDa etc. In some cases, the PEG or PGA-PEG has a molecular weight of no more than 100 kDa, no more than 90 kDa, no more than 80 kDa, no more than 75 kDa, no more than 70 kDa, no more than 60 kDa, no more than 50 kDa, no more than 40 kDa, no more than 30 kDa, no more than 25 kDa, no more than 20 kDa, no more than 10 kDa, no more than 5 kDa, no more than 3 kDa, or no more than 1 kDa. Combinations of any these are also possible, e.g., the PEG or PGA-PEG has a molecular weight between about 1 kDa and about 100 kDa, between about 5 kDa and about 80 kDa, or between about 10 kDa and about 50 kDa, etc.

In some cases, PGA-PEG polymers and water soluble derivatives thereof are available in a variety of degrees of pegylation. This pegylation degree is defined as the percentage of functional groups of PGA or functional groups PGA derivatives are functionalized with PEG. Therefore, appropriate degrees of pegylation PGA-PEG polymer and water soluble derivatives thereof can be, for example, between about 0.1% and about 10%, from about 0.2% to about 5%, about 0.5% to about 2%, or about 0.5%, about 0.6%, about 0.7%, about 0.8%, about 0.9%, about 1%, about 1, 1%, about 1.2%, about 1.3%, about 1.4%, about 1.5%, about 1.6%, about 1.7%, about 1.8%, about 1.9%, or about 2%.

In some embodiments, the proportion of PEG in the PGA-PEG and derivatives water-soluble polymers thereof can be between about 10% and 90% (w/w) relative to the total weight of the polymer, between about 15% and 80%, between about 20% and 70%, or about 20%, about 22%, about 24%, about 26%, about 28%, about 30%, about 32%, about 34%, about 36%, about 38%, about 40%, about 42%, about 44%, about 46%, about 48%, about 50%, about 52%, about 54%, about 56%, about 58%, or about 60%.

In some embodiments, the polymer comprises water-soluble derivatives of PGA or PGA-PEG, where PGA is substituted at one or more available positions, for example amine groups and/or carboxylic acid, with one or more groups, as appropriate. Suitable derivatives of PGA and PGA-PEG derivatives include poly (alquilglutamina) and derivatives PEG-poly (alquilglutamina), such as poly (N-2-(2′-hydroxyethoxy) ethyl-L-glutamine) (PEEG), PEG-PEEG, poly (N-3-(hydroxypropyl)-L-glutamine) (PHPG), PEG-PHPG, poly (N-2-(hydroxyethyl)-L-glutamine) (PHEG), PEG-PHEG, poly(alpha-benzyl-L-glutamate) (PBG), PEG-PBG, poly(gamma-trichloroethyl-L-glutamate) (pTCEG), PEG-pTCEG, poly (dimethylaminoethyl-L-glutamine) (pDMAEG), PEG-pDMAEG, poly(pyridinoethyl-L-glutamine) (pPyAEG), PEG-pPyAEG, poly (aminoethyl-L-glutamine) (PAEG), PEG-PAEG, poly (histamino-L-glutamine) (pHisG), PEG-pHisG, poly (agmatine-L-glutamine) (pAgmG), and PEG-pAgmG, and mixtures thereof.

Polymers—Poly(Aspartic Acid), PASP

In still another set of embodiments, the polymer comprises poly(aspartic acid) (PASP), which is a polymer of aspartic acid, an amino acid, e.g., (PAsp)ₙ. Any number of aspartic acid units are present within the polymer. For example, n is at least 2, at least 4, at least 6, at least 8, at least 10, at least 15, at least 20, at least 25, at least 30, at least 40, at least 50, at least 75, at least 100, at least 200, at least 300, at least 400, or at least 500. In some cases, n is no more than 1000, no more than 500, no more than 200, no more than 100, no more than 50, no more than 30, or no more than 10. Combinations of any of these are also possible, e.g., n is between 2 and 100. It should also be noted that other amino acids is present with in the PASP chain, and the polymer is straight or branched. In addition, as used herein, PASP includes water-soluble salts and water-soluble PASP and/or PASP derivatives.

The poly(aspartic acid) has any suitable molecular weight. For example, the poly(aspartic acid) has a molecular weight of at least 1 kDa, at least 3 kDa, at least 5 kDa, at least 10 kDa, at least 20 kDa, at least 25 kDa, at least 30 kDa, at least 40 kDa, at least 50 kDa, at least 60 kDa, at least 70 kDa, at least 75 kDa, at least 80 kDa, at least 90 kDa, at least 100 kDa etc. In some cases, the poly(aspartic acid) has a molecular weight of no more than 100 kDa, no more than 90 kDa, no more than 80 kDa, no more than 75 kDa, no more than 70 kDa, no more than 60 kDa, no more than 50 kDa, no more than 40 kDa, no more than 30 kDa, no more than 25 kDa, no more than 20 kDa, no more than 10 kDa, no more than 5 kDa, no more than 3 kDa, or no more than 1 kDa. Combinations of any these are also possible, e.g., the poly(aspartic acid) has a molecular weight between about 1 kDa and about 100 kDa, between about 5 kDa and about 80 kDa, or between about 10 kDa and about 50 kDa, etc.

In addition, in some cases, the poly(aspartic acid) is pegylated, e.g., with one or more PEG moieties. The PEG has any of the formulae described herein. For example, the PEG is modified to allow formation of a conjugate PASP-PEG. The modified PEGs are, e.g., as follows:

X¹—(O—CH₂—CH₂)ₙ—X²,

where X¹ is hydrogen or a hydroxyl protecting group blocking the OH radical function for subsequent reactions. For example, n is at least 2, at least 4, at least 6, at least 8, at least 10, at least 15, at least 20, at least 25, at least 30, at least 40, at least 50, at least 75, at least 100, at least 200, at least 300, at least 400, or at least 500. In some cases, n is no more than 1000, no more than 500, no more than 200, no more than 100, no more than 50, no more than 30, or no more than 10. Combinations of any of these are also possible, e.g., n is between 2 and 100.

Polymer Linked to a Hydrophobic Moiety

In some embodiments, the polymer (e.g., PSA) is linked to a hydrophobic moiety. In some cases, the nanoentity is a micelle. In some cases, the nanoentity has an exterior hydrophilic surface and a hydrophobic inner portion. The hydrophobic moiety comprises an alkyl group, for example a straight-chain alkyl group. In some embodiments, the hydrophobic moiety comprises at least 2 carbon atoms. In other embodiments, the hydrophobic moiety comprises at least 3 carbon atoms. In some embodiments, the hydrophobic moiety comprises a C₂-C₂₄ straight-chain alkyl group (e.g., C₂, C₃, C₄, C₅, C₆, C₇, C₈, C₉, C₁₀, Cn, C₁₂, C₁₃, C₁₄, C₁₅, C₁₆, C₁₇, C₁₈, C₁₉, C₂₀, C₂₁, C₂₂, C₂₃, and/or C₂₄). In a particular embodiment, the hydrophobic moiety comprises a straight-chain C₁₂ alkyl group. In some embodiments, the composition of the invention further comprises an aliphatic carbon chain covalently bonded to the polymer (e.g., PSA). In some embodiments, the aliphatic carbon chain comprises a C₂-C₂₄ aliphatic carbon chain (e.g., C₂, C₃, C₄, C₅, C₆, C₇, C₈, C₉, C₁₀, Cn, C₁₂, C₁₃, C₁₄, C₁₅, C₁₆, C₁₇, C₁₈, C₁₉, C₂₀, C₂₁, C₂₂, C₂₃, and/or C₂₄).

Non-limiting examples of hydrophobic moieties include C₃, C₄, C₅, C₆, C₇, C₈, C₉, C₁₀, Cn, C₁₂, C₁₃, C₁₄, C₁₅, C₁₆, C₁₇, C₁₈, C₁₉, C₂₀, C₂₁, C₂₂, C₂₃, C₂₄, or other alkyl group (e.g., a straight-chain or branched alkyl group, e.g., an isoalkyl group). In some cases, the hydrophobic moiety comprises at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, or at least 24 carbon atoms. The hydrophobic moieties are saturated or unsaturated, e.g., containing one or more carbon-carbon double or triple bonds. One technique for attaching a hydrophobic moieties is discussed in Example 7, using C₁₂ as a non-limiting example. In some cases, hydrophobic moieties are attached using activation by a quaternary ammonium salt (e.g., tetrabutylammonium hydroxide) and a tetrafluoroborate (e.g., 2-bromo-1-ethyl pyridinium tetrafluoroborate), prior to reaction with a hydrophobic moiety (e.g., an alkyl amine, such as dodecylamine for C₁₂). Other methods of attaching hydrophobic moieties are also used in other embodiments, for example, using click chemistry, Grignard reactions, or the like.

Additional non-limiting examples of hydrophobic moieties that are added include cycloalkanes (e.g., cyclopropane, cyclobutane, cyclopentane, cylcohexane, etc.), bile salts, terpenoids, terpenes, terpene-derived moieties, and lipophilic vitamins such as vitamins A, D, E, K, and derivatives thereof. Non-limiting examples of bile salts include non-derivatized bile salts such as cholate, deoxycholate, chenodeoxycholate, and ursodeoxycholate, etc. Non-limiting examples of derivatized bile salts include taurocholate, taurodeoxycholate, tauroursodeoxycholate, taurochenodeoxycholate, glycholate, glycodeoxycholate, glycoursodeoxycholate, glycochenodeoxycholate, taurolithocholate, and glycolithocholate, etc.

In another set of embodiments, at least some of the sialic acid, or other monomers of a polymer, are attached to polyethylene glycol (PEG), although it should be understood that PEG is not a requirement in all embodiments. Polyethylene glycol (PEG), in its most common form, is a polymer of the following formula:

H—(O—CH₂—CH₂)ₚ—OH,

where p is an integer representing the PEG polymerization degree. In some cases, the PEG is also modified, e.g., to include:

X³—(O—CH₂—CH₂)p-X⁴,

where X³ is hydrogen or a hydroxyl protecting group blocking the OH function for subsequent reactions. The protective groups of hydroxyl radicals are widely known in the art; representative protecting groups (already including the oxygen to be protected) include, but are not limited to, silyl ethers such as trimethylsilyl ether, triethylsilyl ether, tertbutyldimethylsilyl ether, tert-butyldiphenylsilyl ether, triisopropylsilyl ether, diethylisopropylsilyl ether, tetradimethylsilyl ether, triphenylsilyl ether, di-tert-butylmethylsilyl ether, alkyl ethers such as methyl ether, tert-butyl ether, benzyl ether, p-methoxybenzyl ether, 3,4-dimethoxybenzyl ether, trityl ether, allyl ether; alkoxymethyl ethers such as methoxymethyl ether, 2-methoxyethoxymethyl ether, benzyloxymethyl ether, p-methoxybenzyloxymethyl ether, 2-(trimethylsilyl) ethoxymethyl ether, tetrahydropyranyl ether and related ethers; methylthiomethyl ether, esters such as acetate ester, benzoate ester, ester pivalate, methoxyacetate, chloroacetate ester, levulinate ester, carbonates such as benzyl carbonate, p-nitrobenzyl carbonate, tert-butyl carbonate, 2,2,2-trichloroethyl carbonate, 2-(trimethylsilyl)ethyl, allyl carbonate. In one embodiment, the protecting group is an alkyl ether, such as methyl ether.

X⁴ indicates the anchoring to sialic acid or another monomer of a polymer, and is a covalent bond or a bridge moiety, such as N-hydroxy-succinimide (NHS), maleimide group, biotin, or the like (which may, for example, bind to amines such as primary amines, sulfhydryl moieties, or avidin or streptavidin, respectively, on a modified sialic acid, or other monomer). In some cases, X³ is also a group allowing anchoring, e.g., to sialic acid or another monomer. In addition, in some cases, X³ includes a hydrophobically modified PSA or other polymer, such as is discussed herein. For instance, in one set of embodiments, a hydrophobic moieties, such as C₃, C₄, C₅, C₆, C₇, C₈, C₉, C₁₀, Cn, C₁₂, C₁₃, C₁₄, C₁₅, C₁₆, C₁₇, C₁₈, C₁₉, C₂₀, C₂₁, C₂₂, C₂₃, C₂₄, or another alkyl group (e.g., a straight-chain or branched alkyl group, e.g., an isoalkyl group), attached to the polymer (e.g., PSA), are used. In some cases, the hydrophobic moiety comprises at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, or at least 24 carbon atoms. In some embodiments, the hydrophobic moiety is sufficiently hydrophobic that, compared to unmodified PSA without the X⁴ group, the PSA with X⁴ is more hydrophobic, e.g., partitions to a greater extent in octanol in an octanol/water partitioning system than without the X⁴ group.

In one set of embodiments, the PEGs are attached to the polymer via amine groups and/or carboxylic acid groups. Pegylation can be performed using any suitable method available in the art. See, e.g., Gonzalez and Vaillard, “Evolution of Reactive mPEG Polymers for the Conjugation of Peptides and Proteins,” Curr. Org. Chem., 17(9):975-998, 2013 and Giorgi, et al., “Carbohydrate PEGylation, an approach to improve pharmacological potency,” Beilstein J. Org. Chem., 10:1433-44, 2014. PEGs are available in a variety of molecular weights, and the appropriate molecular weight for a given use is readily determined by a skilled artisan. Thus, for example, a suitable molecular weight of PEG is between about 1 kDa and about 100 kDa, between about 5 kDa and about 80 kDa, or between about 10 kDa and about 50 kDa, e.g., about 10 kDa, about 15 kDa, about 20 kDa, about 25 kDa, about 30 kDa, or about 35 kDa.

In some embodiments, the degree of pegylation is defined as the percentage of functional groups or functional groups in the polymer that are functionalized with PEG. Examples of suitable pegylation grades can be between about 0.1% and about 10%, between about 0.2% and about 5%, or between about 0.5% and about 2%, e.g., about 0.5%, about 0.6%, about 0.7%, about 0.8%; about 0.9%, about 1%, about 1.1%; about 1.2%, about 1.3%, about 1.4%; about 1.5%, about 1.6%, about 1.7%; about 1.8%; about 1.9%; about 2%.

In some embodiments, the proportion of PEG in the final polymer can be between about 10% and 90% (w/w) with respect to the total weight of the polymer, between about 15% and 80%, between about 20% and 70%, or between about 20% and 60%, e.g., about 22%, about 24%, about 26%, about 28%, about 30%, about 32%, about 34%, about 36%, about 38%, about 40%, about 42%, about 44%, about 46%, about 48%, about 50%, about 52%, about 54%, about 56%, about 58%, or about 60%.

Targeting Moiety

In one aspect, the entity also comprises a targeting moiety, although it should be noted that in some embodiments, no targeting moiety is present. The targeting moiety (if present) is used to target delivery of entities, e.g., to certain cell populations within a subject. For instance, the targeting moiety facilitates the access of the nanoentities to one type of cell, e.g., a cancer cell, an endothelial cell, or an immune cell. In some embodiments, the targeting moiety allows targeting of the entity to a specific location within the subject, for example, a specific organ or a specific cell type (e.g., to a tumor or cancer cells). In some cases, the entities are internalized by the cells with no need for targeting moieties, and in some other cases, internalization is facilitated by the targeting moiety (for example, the targeting moiety is a cell-penetrating peptide and/or a tissue-penetrating peptide, for example, Lyp-1 or tLyp-1, or a CendR peptide or other peptides as discussed herein). However, it should be understood that in certain embodiments, the targeting moiety may not necessarily also facilitate internalization. In some embodiments, more than one type of targeting moiety is present. In some embodiments, a targeting moiety includes a cell- and/or tumor/tissue-penetrating peptide.

The subject may be a human or non-human animal. Examples of subjects include, but are not limited to, a mammal such as a cow, sheep, goat, horse, rabbit, pig, mouse, rat, dog, cat, a primate (e.g., a monkey, a chimpanzee, etc.), or the like. In some cases, the subject is a non-mammal such as a bird, an amphibian, or a fish.

A wide variety of targeting moieties may be used in various embodiments. For example, the targeting moieties include peptides, proteins, aptamers, antibodies (including monoclonal antibodies, nanobodies and antibody fragments), nucleic acids, organic molecules, ligands, or the like. Specific non-limiting examples include insulin or transferrin.

For example, in one set of embodiments, the targeting moiety is a peptide, e.g., having a length of no more than 50 amino acids, no more than 40 amino acids, no more than 30 amino acids, or no more than 10 amino acids. In certain embodiments, the targeting moiety comprises a cell-recognition sequence, such as a sequence comprising RGD (arginine-glycine-aspartic acid). In certain embodiments, the targeting moiety comprises a cell-recognition sequence, such as a sequence comprising NGR (asparagine-glycine-arginine).

An “amino acid” is given its ordinary meaning as used in the field of biochemistry. An isolated amino acid typically, but not always (for example, as in the case of proline) has a general structure:

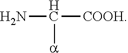

In this structure, alpha (a) is any suitable moiety; for example, alpha (a) is a hydrogen atom, a methyl group, or an isopropyl group. A series of isolated amino acids may be connected to form a peptide or a protein by reaction of the —NH₂ of one amino acid with the —COOH of another amino acid to form a peptide bond (—CO—NH—). In such cases, each of the R groups on the peptide or protein can be referred as an amino acid residue. The “natural amino acids,” as used herein, are the 20 amino acids commonly found in nature, typically in the L-isomer, i.e., alanine (“Ala” or “A”), arginine (“Arg” or “R”), asparagine (“Asn” or “N”), aspartic acid (“Asp” or “D”), cysteine (“Cys” or “C”), glutamine (“Gln” or “Q”), glutamic acid (“Glu” or “E”), glycine (“Gly” or “G”), histidine (“His” or “H”), isoleucine (“Ile” or “I”), leucine (“Leu” or “L”), lysine (“Lys” or “K”), methionine (“Met” or “M”), phenylalaine (“Phe” or “F”), proline (“Pro” or “P”), serine (“Ser” or “S”), threonine (“Thr” or “T”), tryptophan (“Trp” or “W”), tyrosine (“Tyr” or “Y”), and valine (“Val” or “V”).

In one set of embodiments, the targeting moiety is a cell-penetrating and/or a tissue-penetrating peptide. A variety of cell-penetrating peptides are available. For example, the peptide includes a C-terminal “C-end Rule” (CendR) sequence motif (R/K)XX(R/K). A cell-penetrating peptide has the capacity to penetrate a cell membrane. In some cases, the cell-penetrating and/or tissue-penetrating peptide also facilitates the targeting of the nanoentities to the cells. Each X in this sequence is independently an amino acid or no amino acid.

In some cases, the targeting moiety comprises a sequence Z¹X¹X²Z², where Z¹ is R or K, Z² is R or K, and X¹ and X² are each independently an amino acid residue or no amino acid residue. In some cases, one or both ends of the peptide comprise other amino acids, e.g., as in the structures J¹Z¹X¹X²Z², Z¹X¹X²Z²J², or J¹Z¹X¹X²Z²J², wherein each of J¹ and J² is independently an amino acid sequence (e.g., comprising 1, 2, 3, 4, 5, 6, or more amino acid residues) or an or an aliphatic carbon chain. The aliphatic carbon chain contains carbon and hydrogen atoms in any suitable sequence, e.g., straight-chained or branched, and is saturated or unsaturated. For instance, in one set of embodiments, the aliphatic carbon chain is a straight alkyl chain having a formula e.g., —(CH₂)ₙ—, n being 1, 2, 3, 4, 5, 6, 7, 8, 9, 10, 11, 12, or another positive integer. In addition, in some cases, the sequence ends with a cysteine residue, e.g., as in CJ¹Z¹X¹X²Z², CZ¹X¹X²Z²J², or CJ¹Z¹X¹X²Z²J².

Non-limiting examples of CendR peptides include Lyp-1, tLyp-1, iNGR, cLyp1, iRGD, RPARPAR, TT1, or linear TT1. Optionally, other amino acids are present in the peptide as well. Lyp-1 has a sequence CGNKRTRGC (SEQ ID NO: 1). In some embodiments, the two Cys residues are bonded to each other via a disulfide bridge, thereby forming a circular structure. In some cases, only a portion of the Lyp-1 sequence is present, e.g., as in the case of tLyp-1 (CGNKRTR) (SEQ ID NO: 2). cLyp1 has a sequence CGNKRTRGC (SEQ ID NO: 3), where the two cysteines are linked together. iNGR has a sequence CRNGRGPDC (SEQ ID NO: 4), where the two cysteines are linked together. iRGD has a sequence (CRGDKGPDC) (SEQ ID NO: 5) or a sequence CRGDRGPDC (SEQ ID NO:6), where the two cysteines are linked together. RPARPAR has a sequence RPARPAR (SEQ ID NO:7). TT1 has a sequence CKRGARSTC (SEQ ID NO:8), where the two cysteines are linked together. Linear TT1 has a sequence AKRGARSTA (SEQ ID NO:9).

In some embodiments, the targeting moiety comprises a sequence RGD. Optionally, other amino acids may be present in the peptide as well. Non limiting examples of RGD peptides include RGD, RGD-4C, cRGD, or Cilengitide. Optionally, other amino acids may be present in the peptide as well. RGD has a sequence RGD (SEQ ID NO: 10). RGD-4C has a sequence CDCRGDCFC (SEQ ID NO: 11). cRGD has a sequence cRGDf(NMeV) (SEQ ID NO: 12) or c(RGDyK) (SEQ ID NO: 13). Cilengitide has a sequence cyclic-(N-Me-VRGDf-NH) (SEQ ID NO: 14).

In some embodiments, the targeting moiety comprises a sequence NGR. Optionally, other amino acids may be present in the peptide as well.

Some targeting moieties may be seen, for example, in Bertrand N., et al., Cancer Nanotechnology: The impact of passive and active targeting in the era of modern cancer biology, Advanced Drug Delivery Reviews 66 (2014) 2-25, Gilad Y., et al., Recent innovations in peptide based targeted delivery to cancer cells, Biomedicines, 4 (2016) and Zhou G., et al. Aptamers: A promising chemical antibody for cancer therapy, Oncotarget, 7 (2016) 13446-13463. Targeting moieties are selected from, although they are not limited to, peptides, as for example, CendR peptides (e.g. Lyp1, cLyp1, tLyp1, iRGD, iNGR, TT1, linear TT1, RPARPA, F3, etc.), RGD peptides (e.g. 9-RGD, RGD4C, Delta 24-RGD, Delta 24-RGD4C, RGD-K₅, cilengitide, acyclic RGD4C, bicyclic RGD4C, c(RGDfK), c(RGDyK), E-[c(RGDfK)₂], E[c(RGDyK)]₂), NGR peptides, KLWVLPKGGGC (SEQ ID NO: 15), CDCRGDCFC (SEQ ID NO: 16), LABL, angiopeptin-2; proteins, as for example, transferrin, ankyrin repeat protein, affibodies; small molecules, as for example, folic acid, triphenylphosphonium, ACUPA, PSMA, carbohydrate moieties (e.g. mannose, glucose, galactose and their derivatives); and aptamers.

Peptides including any of the sequences disclosed above exhibit, in some embodiments, cell- or tissue-penetrating activity, and particularly in tumor tissue. One set of embodiments is generally directed to the association of cell-penetrating peptides with no targeting properties, e.g., to provide at least some of the nanoentities with cell- or tissue-penetrating activity when non-systemically administered to a subject (e.g. intra-tumoral, nasal, topical, intra-peritoneal, vaginal, rectal, oral, pulmonary, ocular, etc.), or when administered in vitro or ex vivo, e.g., to living cells or tissues. In some cases, some of the polymer (e.g., PSA) is linked to cell-penetrating peptides, e.g. by non-covalent association.

Some cell-penetrating peptides may be found, for example, in Zhang D. et al., Cell-penetrating peptides as noninvasive transmembrane vectors for the development of novel multifunctional drug-delivery systems, Journal of Controlled Release, Volume 229 (2016) Pages 130-139, and Regberg J., et al. Applications of cell-penetrating peptides for tumor targeting and future cancer therapies, Pharmaceuticals, 5 (2012) 991-1007. Cell-penetrating peptides useful for certain embodiments of the present invention are selected from, although they are not limited to, TAT, mTAT (C-5H-TAT-5H-C), G3R6TAT, TAT(49-57), TAT(48-60), MPS, VP22, Antp, gH625, arginine-rich CPPs (e.g. octarginine, polyarginine, stearyl-polyarginine, HIV-1 Rev34-50, FHV coat35-49) penetratin, penetratin-Arg, penetratin-Lys, SR9, HR9, PR9, H(7)K(R(2)), Pep-1, Pep-3, transportan, transportan10, pepFect, pVEC, JB577, TD-1, MPG8, CADY, YTA2, YTA4, SynBl, SynB3, PTD-4, GALA, SPACE, or the like. Cell-penetrating peptides which are coupled with targeting moieties are selected from, although they are not limited to, PEGA (CPGPEGAGC) (SEQ ID NO: 18), CREKA (SEQ ID NO: 19), RVG (YTIWMPENPRPGTPCDIFTNSRGKRASNG) (SEQ ID NO: 20), DV3 (LGASWHRPDKG) (SEQ ID NO: 21), DEVDG (SEQ ID NO: 22), ACPP-MMP-2/9 (PLGLAG) (SEQ ID NO: 23), ACPP-MMP-2 (IAGEDGDEFG) (SEQ ID NO: 24), R8-GRGD (SEQ ID NO: 25), penetratin-RGD, or the like.

Some tumor/tissue-penetrating peptides may be found, for example, in Ruoslahti E., Tumor penetrating peptides for improved drug delivery, Advanced Drug Delivery Reviews, Volumes 110-111 (2017) Pages 3-12. Tumor/tissue-penetrating peptides useful for certain embodiments of the present invention are selected from, although they are not limited to CendR peptides, e.g., iRGD (CRGDKGPDC) (SEQ ID NO: 26), Lyp-1 (CGNKRTRGC) (SEQ ID NO: 27), tLyp-1 (CGNKRTR) (SEQ ID NO: 28), TT1 (CKRGARSTC) (SEQ ID NO: 29), Linear TT1 (AKRGARSTA) (SEQ ID NO: 30), iNGR (CRNGRGPDC) (SEQ ID NO: 31), RPARPAR, F3 (KDEPORRSARLSAKPAPPKPEPKPKKAPAKK) (SEQ ID NO: 32), etc. In one embodiment the tumor/tissue-penetrating peptide comprises a sequence selected from the group consisting of SEQ ID NO: 1 to SEQ ID NO: 22. In a further embodiment the tumor/tissue-penetrating peptide consists of a sequence selected from the group consisting of SEQ ID NO: 1 to SEQ ID NO: 22.

In some cases, antibodies (including nanobodies, antibody fragments, monoclonal antibodies or other antibodies) are attached to a surface or outer shell of an entity, such as a nanocapsule or other entities described herein.

Bond Between the Polymer and the Targeting Moiety

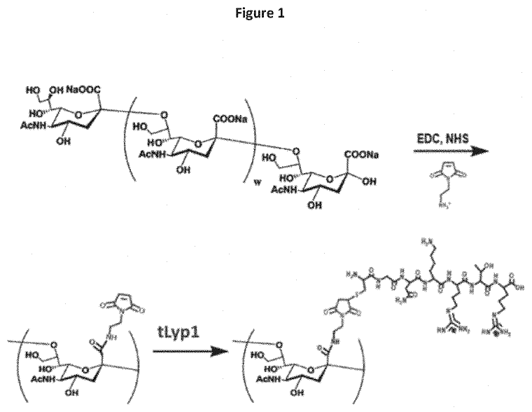

In certain aspects, some of the polymer (e.g., PSA) is bonded to a targeting moiety, e.g., covalently. The polymer is bonded to a targeting moiety directly or indirectly e.g., via a linker, such as an aminoalkyl (C₁-C₄) succinimide linker (including C₁, C₂, C₃, and C₄), or aminoalkyl (C₁-C₄) amido-isopropyl linker (including C₁, C₂, C₃, and C₄). In some cases, other aminoalkylsuccinimide or aminoalkylamido-iso-propyl linkers is used. In some embodiments, the targeting moiety comprises a C terminus, e.g., for binding. In some cases, the aminoalkyl (C₁-C₄) succinimide linker is an aminoethylsuccinimide linker, an aminopropylsuccinimide, an aminobutylsuccinimide, or the like. The aminoalkyl (C₁-C₄) succinimide linker can be created, for example, using an EDC/NHS (l-ethyl-3-(3-dimethylaminopropyl)carbodiimide hydrochloride/A-hydroxysuccinimide) coupling reaction to attach a maleimide moiety to a carboxylic acid moiety on a monomer unit (e.g., a sialic acid unit). In some cases, an N-aminoalkyl (C₁-C₄) maleimide moiety, such as an N-aminoethyl maleimide moiety is reacted with a carboxylic acid moiety on a monomer unit to produce an amide bond, thereby joining the maleimide moiety to the polymer (e.g., PSA). The aminoalkyl (C₁-C₄) amido-iso-propyl linker can be created, for example, using aminoethylmethacrylamide or N-(3-aminopropyl) methacrylamide in the presence of BOP/TBA (benzotriazol-1-yloxy-tris(dimethyl-amino) phosphonium hexafluorophosphate/tetra-n-butylammonium hydroxide). The maleimide moiety or the methacryloyl moiety then can react, e.g., via Michael-type addition, with a cysteine, a thiol group or other sulfur-containing moiety within the peptide to bond the peptide to the polymer (e.g., PSA) via an aminoalkyl (C₁-C₄) succinimide, such as aminoethylsuccinimide linker (see, e.g., FIG. 1), or via an aminoalkyl (C₁-C₄) amido-iso-propyl linker.

In some embodiments, the polymer (e.g., PSA) is bonded to a targeting moiety directly through an amide group. See, e.g., Mojarradi, “Coupling of substances containing a primary amine to hyaluronan via carbodiimide-mediated amidation,” Master's Thesis, Uppsala University, March, 2011. The amide group can be created, for example, by reacting a carboxylic acid moiety on a monomer unit (e.g., a sialic acid unit) and a lysine, arginine or other primary amine-containing moiety within the peptide, in particular the primary amine group is in a lysine or arginine amino acid moiety on the targeting. In some embodiments an activator is present in the reaction to form an intermediate, as for example, a carbodiimide, N-hydroxysuccinimide or DMTMM (4-(4,6-dimethoxy-1,3,5-triazin-2-yl)-4-methylmorpholinium chloride) (Carbohydrate Polymers, 108, (2014), 239-246).

In addition, in certain embodiments of the invention, an entity includes a penetration enhancer able to facilitate cell internalization or tissue-penetration.

Thus, one set of embodiments is generally directed to a method of reacting a carboxylate moiety on a polymer (e.g., PSA) with an aminoalkyl (C₁-C₄) maleimide and/or an aminoalkyl (C₁-C₄) methacrylamide, and reacting the resulting aminoalkyl (C₁-C₄) maleimide and/or the aminoalkyl (C₁-C₄) methacrylamide to a cysteine group on a peptide to produce polymer-aminoalkyl (C₁-C₄) succinimide-peptide and/or a polymer-aminoalkyl (C₁-C₄) aminoisopropyl-peptide composition.

Another set of embodiments is directed to a method of reacting a carboxylate moiety on a polymer (e.g., PSA) with a N-hydroxysuccinimide or a carbodiimide, and reacting the intermediate formed with a lysine or arginine group on a peptide to produce a polymer-amide-peptide.

Pharmaceutical Agents/Drugs

The nanoentities include any of a variety of pharmaceutical agents or drugs, in various embodiments, which may be located internally and/or on the surface of the nanoentities, depending on the embodiment. One, two, three, or more pharmaceutical agents or drugs may be present, e.g., within an inner portion of the nanoentities. For example, the pharmaceutical agent or drug has a size or molecular weight that allows it to be contained within an inner portion of the nanoentity. For example, the pharmaceutical agent or drug is a small molecule, e.g., having a molecular weight of less than 2000 Da. In some cases, the small molecular has a molecular weight of less than 1000 Da. In some embodiments, the molecular weight is less than 500 or 200 Da.

In some cases, the pharmaceutical agents include any substance or mixture of substances intended to be used in the manufacture of a drug product and that, when used in the production of a drug, becomes an active ingredient in the drug product. Such substances furnish pharmacological activity and/or other direct effect in the diagnosis, cure, mitigation, treatment or prevention of disease or to affect the structure and function of the body.

Examples of pharmaceutical agents include any pharmaceutically active chemical or biological compound and any pharmaceutically acceptable salt thereof and any mixture thereof, that provides some pharmacologic effect and is used for treating or preventing a condition. Examples of pharmaceutically acceptable salts include, but are not limited to, hydrochloric, sulfuric, nitric, phosphoric, hydrobromic, maleric, malic, ascorbic, citric, tartaric, pamoic, lauric, stearic, palmitic, oleic, myristic, lauryl sulfuric, naphthalene sulfonic, linoleic, linolenic, and the like. In some cases, the pharmaceutically acceptable salt is a sodium salt, a potassium salt, a lithium salt, a calcium salt, a magnesium salt, an ammonium salt, or the like.

Pharmaceutical agents or drugs can be considered liposoluble, water soluble or amphiphilic (containing both non-polar groups and polar groups simultaneously and tending to form micelles in aqueous media). Given the complexity of classifying pharmaceutical agents or drugs solely on the basis of their solubility, in order to simplify and in no way limit, two classes of drugs are referred to below: liposoluble (compounds with a certain degree of solubility in media containing oils, and/or lipids and/or organic solvents and log P>1.5) and hydrosoluble (compounds with a certain degree of solubility in aqueous medium and log P<1.5), where log P is defined as the octanol-water partition coefficient.

In certain embodiments, the pharmaceutical agents or drugs are liposoluble, e.g., that can be contained within a nonaqueous inner portion of a nanocapsule or other entity, e.g., within an oil, a lipid, and/or organic solvent, for example, an organic solvent mixed with an oil. In addition, in some cases, a liposoluble pharmaceutical agent or drug is present on the outer surface or shell of the entity. Non-limiting examples of organic solvents include, but are not limited to, ethanol, butanol, 2-ethylhexanol, isobutanol, isopropanol, methanol, propanol, propylene glycol, acetone, methyl ethyl ketone, methyl isobutyl ketone, methyl isopropyl ketone, mesityl oxide, trichloroethylene, ethylene bromide, chloroform, ethylene chloride, dichloromethane, tetrachloroethylene, carbon tetrachloride, dimethylformamide, 1,4-dioxane, butyl ether, dimethylformamide ethyl ether, diisopropyl ether, tetrahydrofuran, tert-butyl methyl ether, dimethyl sulfoxide, pyridine, cyclohexane, hexane, acetonitrile, ethyl acetate, toluene, xylene, as well as combinations of these and/or other organic solvents. In some cases, the liposoluble drug is generally hydrophobic in nature, e.g., having a log P greater than 1.5, where P is the intrinsic octanol-water partition coefficient.

Non-limiting examples of liposoluble pharmaceutical agents or drugs which can be used include, but are not limited to, the following: chemotherapeutic or anticancer agents such as taxoids (e.g. docetaxel, paclitaxel, cabazitaxel), tomudex, daunomycin, aclarubicin, bleomycin, dactinomycin, daunorubicin, rapamycin, epirubicin, valrubicin, idarubicin, mitomycin C, mitoxantrone, elesclomol, ingenol mebutate, plicamycin, calicheamicin, esperamicin, degarelix, emtansine, maytansine, maytansinoids (e.g. maytansinoid DM1, maytansinoid 2, maytansinoid DM4), mitomycin, auristatins, vinorelbine, vinblastine, vincristine, vindesine, estramustine, cisplatin hydrophobic derivatives, chlorambucil, bendamustine, carmustine, amantadine, rimantadine, lomustine, semustine, amsacrine, ladribine, cytarabine, (C₁₂-C₁₈)-gemcitabine, tegafur, trimetrexate, epothilones A-E (e.g. sagopilone, ixapebilone, patupilone), eribulin, camptothecins, aminoglutethimide, diaziquone, levamisole, methyl-GAG, mitotane, mitozantrone, testolactone, michellamine B, bryostatin-1, halomon, didemnins (e.g. plitidepsin), trabectedin, lurbinectedin, vorinostat, romidepsin, irinotecan, bortezomib, erlotinib, getifinib, imatinib, vemurafenib, crizotinib, vismodegib, tretinoin, alitretinoin, bexarotene, and the like; or immunomodulators/immunosupressants such as imiquimod, cyclosporin, tacrolimus, pimecrolimus, everolimus, sirolimus, tensirolimus, azathioprine, leflunomide, mycophenolate, and the like; or steroid drugs such as enzalutamide, abiterone, exemestane, fulvestrant, 2-methoxyestradiol, formestane, atamestane, gymnesterol, methyl protodioscin, physalin B, physalin D, physalin F, withaferin A, ginsenosides, azasteroids, cinobufagin, bufalin, dienogest, and the like; or steroidal conjugates with cytotoxic drugs (e.g. nucleosides, paclitaxel, chlorambucil, and metal complexes) such as paclitaxel-estradiol, and the like.

Other illustrative, non-limiting examples of biologically active molecules with liposoluble nature include the following: analgesics and anti-inflammatory agents (e.g. aloxiprin, auranofin, azapropazone, benorylate, diflunisal, etodolac, fenbufen, fenoprofen calcium, flurbiprofen, ibuprofen, indomethacin, ketoprofen, meclofenamic acid, mefenamic acid, nabumetone, naproxen, oxyphenbutazone, phenylbutazone, piroxicam, sulindac, etc.); antihelmintics (e.g., albendazole, bephenium hydroxynaphthoate, cambendazole, dichlorophen, ivermectin, mebendazole, oxamniquine, oxfendazole, oxantel embonate, praziquantel, pyrantel embonate, thiabendazole, etc.); anti-diabetics (e.g., acetohexamide, chlorpropamide, glibenclamide, gliclazide, glipizide, tolazamide, tolbutamide, etc.); anti-depressants (e.g., amoxapine, maprotiline, mianserin, nortriptyline, trazodone, trimipramine, etc.); anti-fungal agents (e.g., amphotericin, butoconazole nitrate, clotrimazole, econazole nitrate, fluconazole, flucytosine, griseofulvin, itraconazole, ketoconazole, miconazole, natamycin, nystatin, sulconazole nitrate, terbinafine, terconazole, tioconazole, undecenoic acid, etc.); anti-malarials (e.g., amodiaquine, chloroquine, chlorproguanil, halofantrine, mefloquine, proguanil, pyrimethamine, quinine sulphate, etc.); anti-migraine agents (e.g., dihydroergotamine, ergotamine, methysergide, pizotifen, sumatriptan, etc.); anti-protozoal agents (e.g., benznidazole, clioquinol, decoquinate, diiodohydroxyquinoline, diloxanide furoate, dinitolmide, furzolidone, metronidazole, nimorazole, nitrofurazone, omidazole, tinidazole, etc.); anti-thyroid agents (e.g., carbimazole, propylthiouracil, etc.); anti-arrhythmic agents (e.g., amiodarone, disopyramide, flecainide acetate, quinidine sulphate, etc.); anti-bacterial agents (e.g., benethamine penicillin, cinoxacin, ciprofloxacin, clarithromycin, clofazimine, cloxacillin, demeclocycline, doxycycline, erythromycin, ethionamide, imipenem, nalidixic acid, nitrofurantoin, rifampicin, spiramycin, sulphabenzamide, sulphadoxine, sulphamerazine, sulphacetamide, sulphadiazine, sulphafurazole, sulphamethoxazole, sulphapyridine, tetracycline, trimethoprim, etc.); anti-coagulants (e.g., dicoumarol, dipyridamole, nicoumalone, phenindione, etc.); anxiolytic, neuroleptics, sedatives, and hypnotics (e.g., alprazolam, amylobarbitone, barbitone, bentazepam, bromazepam, bromperidol, brotizolam, butobarbitone, carbromal, chlordiazepoxide, chlormethiazole, chlorpromazine, clobazam, clotiazepam, clozapine, diazepam, droperidol, ethinamate, flunanisone, flunitrazepam, fluopromazine, flupenthixol decanoate, fluphenazine decanoate, flurazepam, haloperidol, lorazepam, lormetazepam, medazepam, meprobamate, methaqualone, midazolam, nitrazepam, oxazepam, pentobarbitone, perphenazine pimozide, prochlorperazine, sulpiride, temazepam, thioridazine, triazolam, zopiclone, etc.); corticosteroids (e.g., beclomethasone, betamethasone, budesonide, cortisone acetate, desoxymethasone, dexamethasone, fludrocortisone acetate, flunisolide, flucortolone, fluticasone propionate, hydrocortisone, methylprednisolone, prednisolone, prednisone, triamcinolone, etc.); anti-gout agents (e.g., allopurinol, probenecid, sulphinpyrazone, etc.); diuretics (e.g., acetazolamide, amiloride, bendrofluazide, bumetanide, chlorothiazide, chlorthalidone, ethacrynic acid, furosemide, metolazone, spironolactone, triamterene, etc.); beta-blockers (e.g., acebutolol, alprenolol, atenolol, labetalol, metoprolol, nadolol, oxprenolol, pindolol, propranolol, etc.); cardiac inotropic agents (e.g., amrinone, digitoxin, digoxin, enoximone, lanatoside C, medigoxin, etc.); anti-parkinsonian agents (e.g. bromocriptine, lysuride, etc.); histamine-receptor antagonists (e.g., acrivastine, astemizole, cinnarizine, cyclizine, cyproheptadine, dimenhydrinate, flunarizine, loratadine, meclozine, oxatomide, terfenadine, etc.); lipid regulating agents (e.g., bezafibrate, clofibrate, fenofibrate, gemfibrozil, probucol, etc.); nitrates and other anti-anginal agents (e.g., amyl nitrate, glyceryl trinitrate, isosorbide dinitrate, isosorbide mononitrate, pentaerythritol tetranitrate, etc.); nutritional agents (e.g., betacarotene, vitamin A, vitamin B₂, vitamin D, vitamin E, vitamin K, etc.); opioid analgesics (e.g., codeine, dextropropyoxyphene, diamorphine, dihydrocodeine, meptazinol, methadone, morphine, nalbuphine, pentazocine, etc.); sex hormones (e.g., clomiphene citrate, danazol, ethinyl estradiol, medroxyprogesterone acetate, mestranol, methyltestosterone, norethisterone, norgestrel, estradiol, conjugated oestrogens, progesterone, stanozolol, stibestrol, testosterone, tibolone, etc.); and the like. Mixtures of liposoluble drugs may, of course, be used in certain embodiments where therapeutically effective.

In other embodiments, however, the pharmaceutical agents or drugs are hydrosoluble, e.g., that can be contained within an aqueous inner portion of a nanocapsule or associated to the surface of said nanocapsule. In some cases, the hydrosoluble drug exhibits a certain degree of solubility in aqueous medium (e.g., having a log P lower than 1.5, where P is the intrinsic octanol-water partition coefficient). Examples include, but are not limited to, all pharmaceutical acceptable salts of the aforementioned liposoluble drugs, e.g. docetaxel or docetaxel trihydrate; for example, the salt is chloride salt, a sulfate salt, a bromide salt, a mesylate salt, a maleate salt, a citrate salt, a phosphate salt, a hydrochloride salt; a sodium salt, a calcium salt, a potassium salt, a magnesium salt, a meglumine salt, an ammonia salt, etc. In various embodiments, any suitable agent or drug that can be contained within an appropriate solvent within a nanocapsule as discussed herein is used.

Other examples of hydrosoluble pharmaceutical agents or drugs which can be used include, but are not limited to, the following: chemotherapeutic agent (e.g., topotecan, teniposide, etoposide, pralatrexate, omacetaxine, doxorubicin, dacarbazine, procarbazine, hydroxidaunorubicin, hydroxiurea, 6-mercaptopurine, 6-thioguanine, floxuridine or 5-fluorodeoxyuridine, fludarabine, 5-fluorouracil, methotrexate, thiotepa, gemcitabine, pentostatin, mechlorethamine, pibobroman, cyclophosphamide, ifosphamide, busulfan, carboplatin, picoplatin, tetraplatin, satrapalin, platinum-DACH, ormaplatin, oxaplatin, melphalan, aminoglutethimide, etc.); antimicrobial agents (e.g., triclosan, cetylpyridium chloride, domiphen bromide, quaternary ammonium salts, zinc compounds, sanguinarine, fluorides, alexidine, octonidine, EDTA, etc.); non-steroidal anti-inflammatory and pain reducing agents (e.g., aspirin, acetaminophen, ibuprofen, ketoprofen, diflunisal, fenoprofen calcium, flurbiprofen sodium, naproxen, tolmetin sodium, indomethacin, celecoxib, valdecoxib, parecoxib, rofecoxib, etc.); antitussives (e.g., benzonatate, caramiphen edisylate, menthol, dextromethorphan hydrobromide, chlophedianol hydrochloride, etc.); antihistamines (e.g. brompheniramine maleate, chlorpheniramine maleate, carbinoxamine maleate, clemastine fumarate, dexchlorpheniramine maleate, diphenylhydramine hydrochloride, azatadine maleate, diphenhydramine citrate, diphenhydramine hydrochloride, diphenylpyraline hydrochloride, doxylamine succinate, promethazine hydrochloride, pyrilamine maleate, tripelennamine citrate, triprolidine hydrochloride, acrivastine, loratadine, desloratadine, brompheniramine, dexbropheniramine, fexofenadine, cetirizine, montelukast sodium, etc.); expectorants (e.g., guaifenesin, ipecac, potassium iodide, terpin hydrate, etc.); analgesic-antipyretics (e.g., salicylates, phenylbutazone, indomethacin, phenacetin, etc.); anti-migraine drugs (e.g. sumitriptan succinate, zolmitriptan, valproic acid eletriptan hydrobromide, etc.); H₂-antagonists and/or proton pump inhibitors (e.g., ranitidine, famotidine, omeprazole, etc.); or the like.

In some cases, the inner portion can include a peptide, a protein or a nucleotide, many of which are hydrophilic in nature. In addition, in some cases, a hydrosoluble pharmaceutical agent or drug is present on the outer surface or shell of the entity. The peptide, protein or nucleotide has any kind of activity, such as anti-neoplastic, anti-angiogenic, immunomodulatory/immunosuppressive, antigenic, anti-inflammatory, anti-pain, anti-migraine, anti-obesity, anti-diabetic, anti-microbial, wound-healer, anti-helminthic, anti-arrhythmic, anti-viral agents, anti-coagulants, anti-depressant, anti-epileptic, anti-fungal, anti-gout, anti-hypertensive, anti-malarial, anti-muscarinic, anti-protozoal, anti-thyroid, anxiolytic, sedative, hypnotic, neuroleptic, beta-blockers, cardiac inotropic, cell adhesion inhibition, corticosteroid, cytokine receptor activity modulation, diuretic, anti-Parkinson, histamine H-receptor antagonist, keratolytic, lipid regulating, muscle relaxant, anti-anginal, nutritional, stimulant, anti-erectile dysfunction. etc.

Examples of peptides and proteins include, but are not limited to IL-27 interleukin, interferons (e.g. interferon alpha II, interferon alfacon-1, interferon alpha-n3, interferon gamma), Parasporin2, endostatin fragment, macromomycin, actinoxanthin, histidine-rich glycoprotein, carboxypeptidase G2, ribonuclease pancreatic, mitomalcin, arginine deiminase, protein P-30 or onconase, metalloproteinase inhibitor, guanylate kinase, beclin-1, alloferon, ribonuclease mitogillin, aureins, CD276 antigen, dermaseptin-B2, lactoferricin B, plantaricin A, maximins, cecropins, human neutrophil peptides, caerins, nisins, maculatins, mCRAMP, BMAP-27, BMAP-28, citropins, human insulin, recombinant insulin, insulin analogs (e.g., insulin lispro, insulin aspart, insulin glulisine, insulin detemir, insulin degludec, insulin glargine, NPH insulin, etc.), GLP-1 analogs (e.g., exenatide, liraglutide, lixisenatide, albiglutide, dulaglutide, taspoglutide, semaglutide, etc.), GLP-2 analogs (e.g., teduglutide), somatropin, anakinra, domase alpha, whey acidic proteins, SPARC or osteonectin proteins, Protein C, keratin subfamily A, human growth hormone or somatotropin, gonadotropin, angiopoietin, colony-stimulating factors (e.g., macrophage colony-stimulating factor, granulocyte colony-stimulating factor, granulocyte macrophage colony-stimulating factor, etc.), epidermal growth factor, erythropoietin, fibroblast growth factor, GDNF family of ligands, growth differentiation factor-9, hepatocyte growth factor, hepatoma-derived growth factor, insulin-like growth factors, keratinocyte growth factor, macrophage-stimulating protein, neurotrophins, placental growth factor, platelet-derived growth factor, thrombopoietin, transforming growth factors, vascular endothelial growth factor, chemokines, interleukins, lymphokines, tumour necrosis factors (e.g. tumor necrosis factor-alpha), Fc fusion proteins, contulakin-G peptides and derivatives, antiflammins, opioid peptides, lipopeptides (e.g. surotomycin), antigens, such as tetanus and diphtheria toxoids, hepatitis B, and antibodies such as monoclonal antibodies (mAb). Accordingly, as a non-limiting example, a nanoentity such as a nanocapsule contains a monoclonal antibody or a small molecule, e.g., within an inner portion of the entity, within the external portion or in both. Mixtures of hydrosoluble drugs may, of course, be used in certain embodiments, where therapeutically effective.

As used herein, an “antibody” refers to a protein or glycoprotein having one or more polypeptides substantially encoded by immunoglobulin genes or fragments of immunoglobulin genes. The recognized immunoglobulin genes include the kappa, lambda, alpha, gamma, delta, epsilon, and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. A typical immunoglobulin (antibody) structural unit is known to comprise a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (VL) and variable heavy chain (VH) refer to these light and heavy chains respectively. Antibodies exist as intact immunoglobulins or as a number of well characterized fragments produced by digestion with various peptidases.

Thus, for example, pepsin digests an antibody below (i.e. toward the Fc domain) the disulfide linkages in the hinge region to produce F(ab)′2, a dimer of Fab which itself is a light chain joined to VH-CH1 by a disulfide bond. The F(ab)′2 is reduced under mild conditions to break the disulfide linkage in the hinge region thereby converting the (Fab′)2 dimer into an Fab′ monomer. The Fab′ monomer is essentially a Fab with part of the hinge region. While various antibody fragments are defined in terms of the digestion of an intact antibody, these and other fragments are also synthesized de novo, for example, chemically by utilizing recombinant DNA methodology, by “phage display” methods, or the like. Examples of antibodies include single chain antibodies, e.g., single chain Fv (scFv) antibodies in which a variable heavy and a variable light chain are joined together (directly or through a peptide linker) to form a continuous polypeptide. Additional non-limiting examples of antibodies include nanobodies, antibody fragments, monoclonal antibodies, chimeric antibodies, reverse chimeric antibodies, etc. Antigen binding fragments include Fab, Fab′, F(ab)2, dsFv, sFv, unibodies, minibodies, diabodies, tribodies, tetrabodies, nanobodies, probodies, domain bodies, unibodies, bi-specific single-chain variable fragment (bi-scFv), and the like.

Examples of antibodies include, but are not limited to, trastuzumab, bevazizumab, durvalumab, nivolumab, inotuzumab, avelumab, pembrolizumab, olaratumab, atezolizumab, daratumumab, elotuzumab, necitumumab, dinutuximab, blinatumomab, ramucirumab, obinutuzumab, denosumab, ipilimumab, brentuximab, ofatumumab and combinations thereof.

Examples of nucleotides include, but are not limited to, DNA, RNA, siRNA, mRNA, miRNA, PNA, or the like. The nucleotides are sense or antisense in various embodiments.

The pharmaceutical agents are present at up to approximately 50 wt % relative to the total dry weight of the components of the system. However, the appropriate proportion will depend on a variety of factors such as the pharmaceutical agents that is to be incorporated, the indication for which it is used, the efficiency of administration, etc. For example, in some cases, the pharmaceutical agents are present at up to approximately 10 wt %, or up to approximately 5 wt %. In certain embodiments, more than one pharmaceutical agent are present, which can be dissolved in the same solution or separately, depending on the nature of the active pharmaceutical ingredient to be incorporated.

In some embodiments, the nanoentity comprises one or more surfactants. In some embodiments, the nanoentity shell comprises one or more surfactants. In other embodiments, the nanoentity inner portion comprises one or more surfactants. The surfactants (if present) include any of a variety of components that possesses structures and/or functional groups that allow them to interact simultaneously with the lipophilic and hydrophilic part of the formulation. Examples of surfactants include, but are not limited to, the following: polyoxyethylene sorbitan monooleate (polysorbate 80; Tween 80®; HLB 15), polyoxyethylene sorbitan monostearate (Tween® 60, HLB 14.9 and Tween 61®; HLB 9.6), polyoxyethylene sorbitan monooleate (Tween 81®; HLB 10), polyoxyethylene sorbitan tristearate (Tween 65®; HLB 10.5), polyoxyethylene sorbitan trioleate (Tween 85®; HLB 11), polyoxyethylene sorbitan monolaurate (Tween® 20, HLB 16.7 and Tween 21®; HLB 13.3), polyoxyethylene sorbitan monopalmitate (Tween® 40, HLB 15.6); PEGylated fatty acid esters and mixtures with PEG, polyethylene glycol monostearate (HLB 11.6), polyethylene glycol stearate, polyethylene glycol stearate 40 (HLB 17), polyethylene glycol stearate 100 (HLB 18.8), polyethylene glycol dilaurate 400 (HLB 9.7), polyethylene glycol dilaurate 200 (HLB 5.9), polyethylene glycol monopalmitate (HLB 11.6), Kolliphor HS15® (HLB 15), polyethylene glycol-15-hydroxystearate (HLB 14-16), D-alpha-tocopheryl polyethylene glycol succinate (TPGS; HLB 13.2), triethanolammonium oleate (HLB 12), sodium oleate (HLB 18), sodium cholate (HLB 18), sodium deoxycholate (HLB 16), sodium lauryl sulphate (HLB 40), sodium glycocholate (HLB 16-18), triethanolamine oleate (HLB 12), gum tragacanth (HLB 11.9) and sodium dodecyl sulphate (HLB 40); Poloxamer 124 (HLB 16), Poloxamer 188 (HLB 29), Poloxamer 237 (HLB 29), Poloxamer 238 (HLB 28), Poloxamer 278 (HLB 28), Poloxamer 338 (HLB 27), and Poloxamer 407 (HLB 22), sorbitan monooleate (Span® 80, HLB 4.3), sorbitan monolaurate (Span® 20, HLB 8.6), sorbitan monostearate (Span® 60, HLB 4.7), sorbitan trioleate (Span® 85, HLB 1.8), sorbitan sesquioleate (Span® 83, HLB 3.7), sorbitan monopalmitate (Span® 40, HLB 6.7), sorbitan isostearate (Span® 120, HLB 4.7), Lauroyl macrogolglycerides (e.g. Gelucire® 44/14, HLB 14 and Labrafil® M2130CS, HLB 4), Stearoyl macrogolglycerides (e.g. Gelucire® 50/13, HLB 13), Linoleoyl macrogolglycerides (e.g. Labrafil® M2125CS, HLB 4), Oleoyl macrogolglycerides (Labrafil® M1944CS, HLB 4), Caprylocaproyl macrogolglycerides (Labrasol®, HLB 14), lecithins (e.g. egg lecithin, soybean lecithin, non-GMO lecithin, rapeseed lecithin, sunflower lecithin, lysolecithin, etc), phospholipids (e.g. egg phospholipids, soybean phospholipids, synthetic phospholipids, hydrogenated phospholipids, PEGylated phospholipids, phosphatidylcholine, lysophosphaditylcholine, phosphadidylethanolamine, phosphatidylserine, etc.), Phosal®, Phospholipon®, or any combination of any of these and/or other surfactants. In some cases, the surfactant is cationic, e.g., benzethonium choride, benzalkonium chloride, CTAB (hexadecyltrimethylammonium bromide), cetrimide, tetradecyltrimethylammonium bromide, dodecyltrimethylammonium bromide, or the like. In some cases, the cationic surfactant contains an ammonium salt, e.g., as a head group. For example, the head group comprises a primary, secondary, tertiary, or quaternary ammonium salt. In addition, it should be understood that such surfactants are not required in all embodiments.

In some embodiments, the entities comprise at least a cationic surfactant, such as those described above. For instance, certain embodiments of the invention generally directed to nanocapsules may, in some cases, contain surfactants such as cationic surfactants. For instance, certain embodiments of the invention generally directed to nanocapsules that have a targeting moiety may further comprise cationic surfactants.

Methods for Producing Compositions of Entities

Various aspects of the invention are also generally directed to systems and methods for producing compositions such as those described herein, for example, nanoparticles, nanocapsules, micelles, or other nanoentities. In some cases, the composition is a pharmaceutical composition.

As an example, in one set of embodiments, a 1-step solvent diffusion method is used to produce the nanoentities, e.g., nanocapsules. In some cases, this includes preparing an aqueous solution that comprises a polymer (e.g., PSA) and optionally one or more water-soluble surfactants, preparing an oily solution (e.g., comprising an oil and one or more surfactants, and an organic solvent, etc.), and mixing the solutions together. In some cases, the organic solvents are completely or partially evaporated.

In another set of embodiments, a 2-step solvent diffusion method can be used. For instance, in some cases, the method includes preparing an oily solution (e.g., comprising an oil and one or more surfactants and an organic solvent, etc.), and adding it to an aqueous phase (or adding the aqueous phase over the oily phase). The aqueous phase optionally contains one or more water-soluble surfactants. The solutions are stirred to form a nanoemulsion. In some cases, the organic solvent is completely or partially evaporated. Once the nanoemulsion is formed, an aqueous solution that comprises a polymer (e.g., PSA) is added under stirring to produce the nanocapsules.

In yet another set of embodiments, a sonication method is used. For instance, in some cases, the method includes preparing an oily solution, comprising an oil and one or more surfactants and, optionally, an organic solvent, and adding it to an aqueous phase (or adding the aqueous phase over the oily phase). The aqueous phase optionally contains one or more water-soluble surfactants. The solutions are combined while exposed to sonication to form a nanoemulsion. In some cases, the organic solvents are completely or partially evaporated. As previously described for the solvent diffusion method, the polymer (e.g., PSA) is dissolved in the aqueous phase before sonication (1-step nanocapsules formation) or after obtaining the nanoemulsion by sonication (2-step process).

In another embodiment, the present invention relates to method to encapsulate the pharmaceutical agent. In an embodiment, the pharmaceutical agent maybe dissolved in the aqueous phase before preparing the nanoentities. In another embodiment, the pharmaceutical agent maybe incubated with the nanoentities.

In another embodiment, the pharmaceutical agent is a monoclonal antibody which is encapsulated by dissolving it in the aqueous phase before preparing the nanocapsules.

In yet another set of embodiments, a homogenization method is used. For instance, in some cases, the method includes preparing an oily solution, comprising an oil and one or more surfactants, and optionally an organic solvent, and adding it to an aqueous phase (or adding the aqueous phase over the oily phase). The aqueous phase optionally contains one or more water-soluble surfactants. The solutions are combined while homogenizing to form a nanoemulsion. In some cases, the organic solvents are completely or partially evaporated. As previously described for both solvent diffusion and sonication methods, the polymer (e.g., PSA) is dissolved in the aqueous phase before homogenization (1-step nanocapsules formation) or after obtaining the nanoemulsion by homogenization (2-step process).

In another embodiment, a self-emulsifying method is used to produce an emulsion, e.g., as discussed herein. For instance, in some cases, the method includes preparing an oily solution, comprising an oil and one or more surfactants (and optionally a co-solvent) and adding it to an aqueous phase (or adding the aqueous phase over the oily phase). The aqueous phase optionally contains one or more water-soluble surfactants. In one set of embodiments, the emulsion is prepared without the use of co-solvents (e.g., ethanol, PEG, glycerin, propyleneglycol, etc). As previously described, the polymer (e.g., PSA) is dissolved in the aqueous phase before self-emulsification (1-step nanocapsules formation) or after obtaining the nanoemulsion (2-step process).

In another embodiment, the present invention relates to a method for producing nanoentities, comprising an additional step of lyophilization, which may preserve them during storage. In some cases, it is not necessary to use cryoprotectants during lyophilization. In some embodiments, it is not necessary to dilute the colloidal system before lyophilization, since the nanoentities do not form aggregates during reconstitution of the lyophilizate. In some cases, it is possible to add one or more sugars, for example, sugars that exert a cryoprotectant effect. Examples of cryoprotectants include, but are not limited to, the following: trehalose, glucose, sucrose, mannitol, maltose, polyvinyl pyrrolidone (PVP), glycerol, polyethylene glycol (PEG), propylene glycol, 2-methyl-2,4-pentanediol (MPD), raffinose, dextran, fructose, stachyose, or the like. In some cases, scryoprotectants or other additives have other effects, e.g., as buffers to control pH. In lyophilized form, the nanoentities are stored for long periods of time, and can be regenerated, for example, by adding water.

Administration of the Compositions

Another aspect provides a method of administering any composition discussed herein to a living organism. When administered, the compositions of the invention are applied in a therapeutically effective amount as a pharmaceutically acceptable formulation. As used herein, the term “pharmaceutically acceptable” means that the formulation contains agents or excipients compatible with the form required for administration to a living organism, without causing deleterious effects. Any of the compositions of the present invention are administered to the living organism in a therapeutically effective dose. A “therapeutically effective” or an “effective” as used herein means that amount necessary to delay the onset of, inhibit the progression of, halt altogether the onset or progression of, diagnose a particular condition being treated, or otherwise achieve a medically desirable result. The terms “treat,” “treated,” “treating” and the like, generally refer to administration of the inventive compositions to a living organism. When administered to a living organism, effective amounts will depend on the particular condition being treated and the desired outcome. A therapeutically effective dose is determined by those of ordinary skill in the art, for instance, employing factors such as those further described below and using no more than routine experimentation. For example, in one embodiment, the compositions are used herein to treat cancer, e.g., through administration of docetaxel to the living organism, e.g., intravenously.

Some embodiments of the invention are generally directed to the use of a composition as disclosed herein for the preparation of a medicament. For instance, certain embodiments refer to the compositions disclosed herein for use in the treatment of cancer.

In administering the compositions of the invention to a living organism, dosing amounts, dosing schedules, routes of administration, and the like are selected so as to affect known activities of these compositions. Dosages are estimated based on the results of experimental models, optionally in combination with the results of assays of compositions of the present invention. Dosage are adjusted appropriately to achieve desired drug levels, local or systemic, depending upon the mode of administration. The doses are given in one or several administrations per day, week, or month.

The dose of the composition to the living organism is such that a therapeutically effective amount of the composition reaches the active site of the composition within the living organism. The dosage is given in some cases at the maximum amount while avoiding or minimizing any potentially detrimental side effects within the living organism. The dosage of the composition that is administered is dependent upon factors such as the final concentration desired at the active site, the method of administration to the living organism, the efficacy of the composition, the permanence of the composition within the living organism, the timing of administration, the effect of concurrent treatments. The dose delivered may also depend on conditions associated with the living organism, and can vary from organism to organism in some cases. For example, the age, sex, weight, size, environment, physical conditions, or current state of health of the living organism may also influence the dose required and/or the concentration of the composition at the active site. Variations in dosing may occur between different individuals or even within the same individual on different days. In some cases, a maximum dose is used, that is, the highest safe dose according to sound medical judgment. In some cases, the dosage form is such that it does not substantially deleteriously affect the living organism.

In certain embodiments, a composition of the invention is administered to a living organism who has cancer. Administration of a composition of the invention is accomplished by any medically acceptable method which allows the composition to reach its target. The particular mode selected will depend of course, upon factors such as those previously described, for example, the particular composition, the severity of the state of the living organism being treated, the dosage required for therapeutic efficacy, etc. As used herein, a “medically acceptable” mode of treatment is a mode able to produce effective levels of the composition within the living organism without causing clinically unacceptable adverse effects.

Any medically acceptable method is used to administer the composition to the living organism. The administration is localized (i.e., to a particular region, physiological system, tissue, organ, or cell type) or systemic, depending on the condition to be treated. For example, the composition is administered orally, or through other techniques such as vaginally, rectally, buccally, pulmonary, topically, nasally, transdermally, intratumorally, through parenteral injection or implantation, via surgical administration, or any other method of administration where access to the target by the composition of the invention is achieved. Compositions suitable for oral administration are presented as discrete units such as hard or soft capsules, pills, sachets, tablets, troches, or lozenges, each containing a predetermined amount of the active compound. Other oral compositions suitable for use with the invention include solutions or suspensions in aqueous or non-aqueous liquids such as a syrup, an elixir, or an emulsion. In another set of embodiments, the composition is used to fortify a food or a beverage. Rectal administration can be used in some embodiments, for example, in the form of an enema, suppository, or foam.

In one set of embodiments, the administration of the composition is parenteral, intratumoral, or oral. In some embodiments, the composition is administered by injection or infusion. In one embodiment, the injection is selected from intratumoral, subcutaneous, intramuscular, or intravenous injection. In another embodiment, the composition is administered through intrathecal injection or infusion.

In certain embodiments of the invention, the administration of a composition of the invention is designed so as to result in sequential exposures to a composition over a certain time period, for example, hours, days, weeks, months, or years. This is accomplished, for example, by repeated administrations of a composition of the invention by one of the methods described above. Administration of a composition can be alone, or in combination with other therapeutic agents and/or compositions.

In certain embodiments of the invention, a composition can be combined with a suitable pharmaceutically acceptable carrier, for example, as incorporated into a polymer release system, or suspended in a liquid, e.g., in a dissolved form or a colloidal form. In general, pharmaceutically acceptable carriers suitable for use in the invention are well-known to those of ordinary skill in the art. As used herein, a “pharmaceutically acceptable carrier” refers to a non-toxic material that does not significantly interfere with the effectiveness of the biological activity of the active compound(s) to be administered, but is used as a formulation ingredient, for example, to stabilize or protect the active compound(s) within the composition before use. The term “carrier” denotes an organic or inorganic ingredient, which is natural or synthetic, with which one or more active compounds of the invention are combined to facilitate the application of a composition as discussed herein. The carrier is co-mingled or otherwise mixed with one or more compositions of the present invention, and with each other, in a manner such that there is no interaction which would substantially impair the desired pharmaceutical efficacy. The carrier is either soluble or insoluble, depending on the application. Examples of well-known carriers include glass, polystyrene, polypropylene, polyethylene, dextran, nylon, amylase, natural and modified cellulose, polyacrylamide, agarose and magnetite. The nature of the carrier can be either soluble or insoluble. Those skilled in the art will know of other suitable carriers, or will be able to ascertain such, using only routine experimentation.

In some embodiments, a composition of the invention can include pharmaceutically acceptable carriers with formulation ingredients such as salts, carriers, buffering agents, emulsifiers, diluents, excipients, chelating agents, fillers, drying agents, antioxidants, antimicrobials, preservatives, binding agents, bulking agents, silicas, solubilizers, or stabilizers that are used with the active compound. For example, if the formulation is a liquid, the carrier may be a solvent, partial solvent, or non-solvent, and may be aqueous or organically based. Examples of suitable formulation ingredients include diluents such as calcium carbonate, sodium carbonate, lactose, kaolin, calcium phosphate, or sodium phosphate; granulating and disintegrating agents such as corn starch or alginic acid; binding agents such as starch, gelatin or acacia; lubricating agents such as magnesium stearate, stearic acid, or talc; time-delay materials such as glycerol monostearate or glycerol distearate; suspending agents such as sodium carboxymethylcellulose, methylcellulose, hydroxypropylmethylcellulose, sodium alginate, polyvinylpyrrolidone; dispersing or wetting agents such as lecithin or other naturally-occurring phosphatides; thickening agents such as cetyl alcohol or beeswax; buffering agents such as acetic acid and salts thereof, citric acid and salts thereof, boric acid and salts thereof, or phosphoric acid and salts thereof; or preservatives such as benzalkonium chloride, chlorobutanol, parabens, or thimerosal. Suitable carrier concentrations can be determined by those of ordinary skill in the art, using no more than routine experimentation. A composition as discussed herein can be formulated into preparations in solid, semi-solid, liquid or gaseous forms such as tablets, capsules, elixirs, powders, granules, ointments, solutions, depositories, inhalants or injectables. Those of ordinary skill in the art will know of other suitable formulation ingredients, or will be able to ascertain such, using only routine experimentation.

Preparations include sterile aqueous or non-aqueous solutions, suspensions and emulsions, which can be isotonic with the blood of the living organism in certain embodiments. Examples of non-aqueous solvents are polypropylene glycol, polyethylene glycol, vegetable oil such as olive oil, sesame oil, coconut oil, peanut oil, mineral oil, injectable organic esters such as ethyl oleate, or fixed oils including synthetic mono or di-glycerides. Aqueous carriers include water, alcoholic/aqueous solutions, emulsions or suspensions, including saline and buffered media. Parenteral vehicles include sodium chloride solution, 1,3-butandiol, Ringer's dextrose, dextrose and sodium chloride, lactated Ringer's or fixed oils. Intravenous vehicles include fluid and nutrient replenishers, electrolyte replenishers (such as those based on Ringer's dextrose), and the like. Preservatives and other additives are also present such as, for example, antimicrobials, antioxidants, chelating agents and inert gases and the like. Those of skill in the art can readily determine the various parameters for preparing and formulating a composition as discussed herein without resort to undue experimentation.

The present invention also provides any of the above-mentioned compositions in kits, optionally including instructions for use of the composition for the treatment of cancer or other diseases. Instructions also may be provided for administering a composition by any suitable technique as previously described, for example, orally or intravenously.

The compositions of the invention may be in the form of a kit. The kit typically defines a package including any one or a combination of compositions of the invention and other ingredients as previously described. The kits also can include other containers with one or more solvents, surfactants, preservative and/or diluents (e.g., normal saline (0.9% NaCl), or 5% dextrose) as well as containers for mixing, diluting or administering the composition to a living organism.

The compositions of the kit may be provided as liquid solutions or as dried powders. When a composition provided is a dry powder, the composition may be reconstituted by the addition of a suitable solvent. In embodiments where liquid forms of a composition are used, the liquid form may be concentrated or ready to use. The solvent will depend on a composition and the mode of use or administration.

Spanish Application Serial No. P201731277, filed on 2 Nov. 2017, entitled “Sistemas de Liberation de Farmacos de Acido Polisialico y Metodos” is incorporated herein by reference in its entirety in the U.S. and other countries where applicable.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of”.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

When the word “about” is used herein in reference to a number, it should be understood that still another embodiment of the invention includes that number not modified by the presence of the word “about”.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising”, “including”, “carrying”, “having”, “containing,” “involving”, “holding”, “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

### Aspects/Embodiments of the Invention in so-Called Claim Format

1. (Aspect 1): A composition, comprising: a plurality of nanoentities comprising an inner portion surrounded by an outer shell, the outer shell comprising polysialic acid, the inner portion comprising at least one hydrophobic compound.
2. The composition of claim 1, wherein at least some of the plurality of nanoentities further comprises a targeting moiety and/or a cell-penetrating peptide and/or a tumor/tis sue-penetrating peptide.
3. The composition of claim 2, wherein the targeting moiety is bonded to the polysialic acid electrostatically.
4. The composition of claim 2, wherein the targeting moiety is bonded to the polysialic acid via a linker.
5. The composition of claim 2, wherein the targeting moiety is bonded to the polysialic acid via an aminoalkyl (C₁-C₄) maleimide linker, an aminoalkyl (C₁-C₄) methacrylamide linker, or directly through an amide group.
6. The composition of claim 5, wherein the aminoalkyl (C₁-C₄) maleimide linker is created via an EDC/NHS (1-ethyl-3-(3-dimethylaminopropyl)carbodiimide hydrochloride/A-hydroxysuccinimide) or via a DMTMM (4-(4,6-dimethoxy-1,3,5-triazin-2-yl)-4-methylmorpholinium chloride) coupling reaction.
7. The composition of claim 5, wherein the targeting moiety is bonded to the polysialic acid via an aminoethylmaleimide linker.
8. The composition of any one of claims 2-7, wherein the targeting moiety comprises a peptide or a protein.
9 The composition of any one of claims 2-8, wherein the targeting moiety comprises an aptamer.
10. The composition of any one of claims 2-9, wherein the targeting moiety comprises a nucleic acid.
11. The composition of any one of claims 2-10, wherein the targeting moiety comprises an antibody or a fragment thereof.
12. The composition of any one of claims 2-11, wherein the targeting moiety comprises a nanobody, a unibody, a minibody, a diabody, a tribody, and/or a tetrabody.
13. The composition of any one of claims 2-12, wherein the targeting moiety comprises an organic molecule.
14. The composition of any one of claims 2-13, wherein the targeting moiety comprises a ligand.
15. The composition of any one of claims 2-14, wherein the targeting moiety comprises a cell-penetrating peptide.
16. The composition of claim 15, wherein the cell-penetrating peptide is chemically linked to polysialic acid.
17. The composition of any one of claims 2-16, wherein the targeting moiety comprises a CendR peptide.
18. The composition of any one of claims 2-17, wherein the targeting moiety comprises an amino acid sequence Z¹X¹X²Z², wherein Z¹ is R or K, Z² is R or K, and X¹ and X² are each an amino acid residue.
19. The composition of any one of claims 2-18, wherein the targeting moiety comprises an amino acid sequence RGD.
20. The composition of any one of claims 2-19, wherein the targeting moiety comprises an amino acid sequence NGR.
21. The composition of any one of claims 2-20, wherein the targeting moiety comprises an amino acid sequence CJ¹Z¹X¹X²Z², wherein J¹ is an amino acid sequence.
22. The composition of any one of claims 2-21, wherein the targeting moiety comprises an amino acid sequence J¹RGD, wherein J¹ is an amino acid sequence.
23. The composition of any one of claims 2-22, wherein the targeting moiety comprises an amino acid sequence J¹Z¹X¹X²Z²J², wherein each of J¹ and J² is independently an amino acid sequence.
24. The composition of any one of claims 2-23, wherein the targeting moiety comprises an amino acid sequence J¹RGDJ², wherein each of J¹ and J² is independently an amino acid sequence.
25. The composition of any one of claims 2-24, wherein the targeting moiety comprises an amino acid sequence CJ¹Z¹X¹X²Z²J², wherein each of J¹ and J² is independently an amino acid sequence.
26. The composition of any one of claims 2-25, wherein the targeting moiety comprises Lyp-1.
27. The composition of any one of claims 2-26, wherein the targeting moiety comprises tLyp-1.
28. The composition of any one of claims 2-27, wherein the targeting moiety comprises cLyp1.
29. The composition of any one of claims 2-28, wherein the targeting moiety comprises iNGR.
30. The composition of any one of claims 2-29, wherein the targeting moiety comprises iRGD.
31. The composition of any one of claims 2-30, wherein the targeting moiety comprises RPARPAR.
32. The composition of any one of claims 2-31, wherein the targeting moiety comprises TT1.
33. The composition of any one of claims 2-32, wherein the targeting moiety comprises linear TT1.
34. The composition of any one of claims 2-33, wherein the targeting moiety comprises RGD-4C.
35. The composition of any one of claims 2-34, wherein the targeting moiety comprises cRGD.
36. The composition of any one of claims 2-35, wherein the targeting moiety comprises Cilengitide.
37. The composition of any one of claims 2-36, wherein the targeting moiety is selected from the group consisting of Lyp1, tLyp1, cLyp1, iNGR, iRGD, RPARPAR, TT1, linear TT1, RGD-4C, cRGD, Cilengitide, F3, 9-RGD, RGD4C, Delta 24-RGD, Delta 24-RGD4C, RGD-K5, acyclic RGD4C, bicyclic RGD4C, c(RGDfK), c(RGDyK), E-[c(RGDfK)2], E[c(RGDyK)]2, KLWVLPKGGGC, CDCRGDCFC, LABL, angiopeptin-2, antibodies, nanobodies, transferrin, ankyrin repeat protein, affibodies, folic acid, triphenylphosphonium, ACUPA, PSMA, carbohydrate moieties and aptamers.
38. The composition of any one of claims 1-37, wherein the outer shell further comprises a penetration enhancer.
39. The composition of any one of claims 1-38, wherein at least some of the polysialic acid is linked to a hydrophobic moiety.
40. The composition of claim 39, wherein the hydrophobic moiety is selected from an alkyl group, cycloalkanes, bile salts and derivatives, terpenoids, terpenes, terpene-derived moieties and lipophilic vitamins.
41. The composition of any one of claim 39 or 40, wherein the hydrophobic moiety comprises a straight-chain alkyl group.
42. The composition of any one of claims 39-41, wherein the hydrophobic moiety comprises at least 2 carbon atoms.
43. The composition of any one of claims 39-42, wherein the hydrophobic moiety comprises at least 3 carbon atoms.
44. The composition of any one of claims 39-43, wherein the hydrophobic moiety comprises a C₂-C₂₄ straight-chain alkyl group.
45. The composition of any one of claims 39-44, wherein the hydrophobic moiety comprises a straight-chain C₁₂ alkyl group.
46. The composition of any one of claims 1-45, further comprising an aliphatic carbon chain covalently bonded to the polysialic acid.
47. The composition of claim 46, wherein the aliphatic carbon chain comprises a C₂-C₂₄ aliphatic carbon chain.
48. The composition of any one of claims 1-47, wherein at least about 90 wt % of the outer shell comprises polysialic acid.
49. The composition of any one of claims 1-48, wherein at least some of the plurality of nanoentities are substantially solid.
50. The composition of any one of claims 1-49, wherein at least some of the plurality of nanoentities are nanocapsules.
51. The composition of any one of claims 1-50, wherein at least some of the polysialic acid comprises N-acetylneuraminic acid.
52. The composition of any one of claims 1-51, wherein at least some of the polysialic acid comprises 2-keto-3-deoxynonic acid.
53. The composition of any one of claims 1-52, wherein at least some of the polysialic acid comprises lactaminic acid.
54. The composition of any one of claims 1-53, wherein at least some of the polysialic acid comprises N-sialic acid.
55. The composition of any one of claims 1-54, wherein at least some of the polysialic acid comprises O-sialic acid.
56. The composition of any one of claims 1-55, wherein at least some of the polysialic acid comprises at least 2 sialic acid units.
57. The composition of any one of claims 1-56, wherein at least some of the polysialic acid comprises at least 4 sialic acid units.
58. The composition of any one of claims 1-57, wherein at least some of the polysialic acid comprises at least 8 sialic acid units.
59. The composition of any one of claims 1-58, wherein at least some of the polysialic acid comprises sialic acid units bonded via 2->8 bonding.
60. The composition of any one of claims 1-59, wherein at least some of the polysialic acid comprises sialic acid units bonded via 2->9 bonding.
61. The composition of any one of claims 1-60, wherein the inner portion is nonaqueous.
62. The composition of any one of claims 1-61, wherein the inner portion comprises a pharmaceutical agent.
63. The composition of claim 62, wherein the pharmaceutical agent is liposoluble.
64. The composition of claim 62, wherein the pharmaceutical agent is amphiphilic.
65. The composition of claim 62, wherein the pharmaceutical agent is hydrosoluble.
66. The composition of claim 62, wherein the pharmaceutical agent is a monoclonal antibody.
67. The composition of claim 62, wherein the pharmaceutical agent is a polynucleotide.
68. The composition of claim 62, wherein the pharmaceutical agent is docetaxel.
69. The composition of claim 62, wherein the pharmaceutical agent is an anticancer drug.
70. The composition of claim 69, wherein the pharmaceutical agent is selected from the group consisting of gemcitabine, paclitaxel, cabazitaxel, tomudex, daunomycin, aclarubicin, bleomycin, dactinomycin, daunorubicin, rapamycin, epirubicin, valrubicin, idarubicin, mitomycin C, mitoxantrone, elesclomol, ingenol mebutate, plicamycin, calicheamicin, esperamicin, degarelix, emtansine, maytansine, maytansinoid DM1, maytansinoid 2, maytansinoid DM4, mitomycin, auristatins, vinorelbine, vinblastine, vincristine, vindesine, estramustine, cisplatin hydrophobic derivatives, chlorambucil, bendamustine, carmustine, amantadine, rimantadine, lomustine, semustine, amsacrine, ladribine, cytarabine, (C₁₂-C₁₈)-gemcitabine, tegafur, trimetrexate, sagopilone, ixapebilone, patupilone, eribulin, camptothecin, aminoglutethimide, diaziquone, levamisole, methyl-GAG, mitotane, mitozantrone, testolactone, michellamine B, bryostatin-1, halomon, didemnins, plitidepsin, trabectedin, lurbinectedin, vorinostat, romidepsin, irinotecan, bortezomib, erlotinib, getifinib, imatinib, vemurafenib, crizotinib, vismodegib, tretinoin, alitretinoin, bexarotene, tacrolimus, everolimus, topotecan, teniposide, etoposide, pralatrexate, omacetaxine, doxorubicin, dacarbazine, procarbazine, hydroxidaunorubicin, hydroxiurea, 6-mercaptopurine, 6-thioguanine, floxuridine or 5-fluorodeoxyuridine, fludarabine, 5-fluorouracil, methotrexate, thiotepa, pentostatin, mechlorethamine, pibobroman, cyclophosphamide, ifosphamide, busulfan, carboplatin, picoplatin, tetraplatin, satrapalin, platinum-DACH, ormaplatin, oxaplatin, melphalan, aminoglutethimide, trastuzumab, bevazizumab, durvalumab, nivolumab, inotuzumab, avelumab, pembrolizumab, olaratumab, atezolizumab, daratumumab, elotuzumab, necitumumab, dinutuximab, blinatumomab, ramucirumab, obinutuzumab, denosumab, ipilimumab, brentuximab, ofatumumab and combinations thereof.
71. The composition of any one of claims 62-70, wherein the inner portion comprises at least two pharmaceutical agents.
72. The composition of any one of claims 1-71, wherein the outer shell comprises a pharmaceutical agent.
73. The composition of claim 72, wherein the pharmaceutical agent of the outer shell is lipo soluble.
74. The composition of claim 72, wherein the pharmaceutical agent of the outer shell is amphiphilic.
75. The composition of claim 72, wherein the pharmaceutical agent of the outer shell is hydro soluble.
76. The composition of claim 72, wherein the pharmaceutical agent of the outer shell is a polynucleotide.
77. The composition of any one of claims 1-76, wherein the plurality of nanoentities have an average diameter of less than 1 micrometer.
78. The composition of any one of claims 1-77, wherein the plurality of nanoentities have an average diameter of less than 250 nm.
79. The composition of any one of claims 1-78, wherein the plurality of nanoentities have an average diameter of less than 150 nm.
80. The composition of any one of claims 1-79, wherein the plurality of nanoentity comprises a micelle.
81. The composition of any one of claims 1-80, with the proviso that the plurality of nanoentities are not liposomes.
82. The composition of any one of claims 1-81, with the proviso that the plurality of nanoentities does not include protamine.
83. The composition of any one of claims 1-82, with the provision that the plurality of nanoentities does not include polyarginine.
84. The composition of any one of claims 1-83, wherein the plurality of nanoentities does not comprise more than one outer shell.
85. (Aspect 2): Composition according to any one of claims 1-84 for use as a medicament.
86. A method, comprising administering the composition of any one of claims 1-85 to a living organism.
87. The method of claim 86, wherein the living organism is a human.
88. (Aspect 3): A method, comprising: reacting a carboxylate moiety on a polysialic acid with an aminoalkyl (C₁-C₄) maleimide and/or an aminoalkyl (C₁-C₄) methacrylamide; and reacting the resulting aminoalkyl (C₁-C₄) maleimide and/or the aminoalkyl (C₁-C₄) methacrylamide to a thiol group on a targeting moiety to produce a polysialic acid-aminoalkyl (C₁-C₄) succinimide-peptide and/or a polysialic acid-aminoalkyl (C₁-C₄) amidoisopropyl-peptide composition.
89. The method of claim 88, further comprising forming an emulsion comprising the polysialic acid-aminoalkyl (C₁-C₄) succinimide-peptide composition and/or the polysialic acid-aminoalkyl (C₁-C₄) amidoisopropyl-peptide composition; and forming a plurality of nanoparticles from the emulsion.
90. The method of claim 89, wherein at least some of the plurality of nanoparticles comprise an inner portion surrounded by an exposed outer shell.
91. (Aspect 4): A method, comprising: reacting a carboxylate moiety on a polysialic acid with a N-hydroxysuccinimide and/or a carbodiimide to form an intermediate; and reacting the intermediate with a lysine or arginine group on a targeting moiety to produce a polysialic acid-amide-peptide.
92. The method of claim 91, further comprising forming an emulsion comprising the polysialic acid-amide-peptide; and forming a plurality of nanoparticles from the emulsion.
93. The method of claim 92, wherein at least some of the plurality of nanocapsules comprise an inner portion surrounded by an exposed outer shell.
94. (Aspect 5): A composition, comprising: a plurality of nanoentities comprising an inner portion surrounded by an outer shell, the outer shell comprising polysialic acid, at least some of the nanoentities further comprising a monoclonal antibody contained within the inner portion.
95. The composition of claim 94, wherein the monoclonal antibody is not exposed externally of the nanoentities.
96. The composition of any one of claim 94 or 95, wherein at least some of the plurality of nanoentities further comprises one or more surfactants.
97. The composition of any one of claims 94-96, wherein at least some of the plurality of nanoentities further comprises a targeting moiety and/or a cell penetrating peptide and/or a tumor/tissue penetrating peptide.
98. The composition of claim 97, wherein the targeting moiety is bonded to the polysialic acid electrostatically.
99. The composition of claim 97, wherein the targeting moiety is bonded to the polysialic acid via a linker.
100. The composition of claim 97, wherein the targeting moiety is bonded to the polysialic acid via an aminoalkyl (C₁-C₄) succinimide linker, an aminoalkyl (C₁-C₄) amide-iso-propyl linker, or directly through an amide group.
101. The composition of claim 100, wherein the aminoalkyl (C₁-C₄) succinimide linker is created via an EDC/NHS (l-ethyl-3-(3-dimethylaminopropyl)carbodiimide hydrochloride/A-hydroxysuccinimide) coupling reaction.
102. The composition of claim 99, wherein the targeting moiety is bonded to the polysialic acid via an aminoethylsuccinimide linker.
103. The composition of any one of claims 97-102, wherein the targeting moiety comprises a cell-penetrating peptide.
104. The composition of claim 103, wherein the cell-penetrating peptide is chemically linked to polysialic acid.
105. The composition of any one of claims 97-104, wherein the targeting moiety comprises an amino acid sequence RGD.
106. The composition of any one of claims 97-105, wherein the targeting moiety comprises an amino acid sequence NGR.
107. The composition of any one of claims 97-106, wherein the targeting moiety comprises Lyp-1.
108. The composition of any one of claims 97-107, wherein the targeting moiety comprises tLyp-1.
109. The composition of any one of claims 97-108, wherein the targeting moiety comprises cLyp1.
110. The composition of any one of claims 94-109, wherein the outer shell further comprises a penetration enhancer.
111. The composition of any one of claims 94-109, wherein at least some of the polysialic acid is linked to a hydrophobic moiety.
112. The composition of any one of claims 94-111, wherein at least about 93 wt % of the outer shell comprises polysialic acid.
113. The composition of any one of claims 94-112, wherein at least some of the plurality of nanoentities are nanocapsules.
114. The composition of any one of claims 94-113, wherein the plurality of nanoentities have an average diameter of less than 1 micrometer.
115. The composition of any one of claims 94-114, wherein the plurality of nanoentities have an average diameter of less than 250 nm.
116. The composition of any one of claims 94-115, wherein the plurality of nanoentities have an average diameter of less than 150 nm.
117. The composition of any one of claims 94-116, with the provision that the plurality of nanoentities are not liposomes.
118. The composition of any one of claims 94-117, with the provision that the plurality of nanoentities does not include protamine.
119. The composition of any one of claims 94-118, with the provision that the plurality of nanoentities does not include polyarginine.
120. The composition of any one of claims 94-119, wherein the plurality of nanoentities do not comprise more than one outer shell.
121. (Aspect 6): Composition according to any one of claims 94-120 for use as a medicament.
122. A method, comprising administering the composition of any one of claims 94-120 to a living organism.
123. (Aspect 7): A composition, comprising: a plurality of nanoentities comprising an inner portion surrounded by an outer shell, the outer shell consisting essentially of polysialic acid, the inner portion comprising at least one hydrophobic compound.
124. The composition of claim 123, wherein the outer shell is at least 90 wt % polysialic acid.
125. The composition of any one of claim 123 or 124, wherein at least some of the plurality of nanoentities further comprise a surfactant positioned between the inner portion and the outer shell.
126. The composition of any one of claims 123-125, wherein at least some of the plurality of nanoentities further comprise a targeting moiety comprising a cell-penetrating peptide chemically linked to the polysialic acid.
127. The composition of claim 126, wherein the targeting moiety comprises a CendR peptide.
128. The composition of any one of claim 126 or 127, wherein the targeting moiety comprises tLyp-1.
129. The composition of any one of claims 126-128, wherein the targeting moiety is bonded to the polysialic acid via a linker.
130. The composition of any one of claims 126-129, wherein the targeting moiety is bonded to the polysialic acid via an aminoalkyl (C₁-C₄) succinimide linker.
131. The composition of any one of claims 123-130, with the proviso that the plurality of nanoentities are not liposomes.
132. The composition of any one of claims 123-131, with the provision that the plurality of nanoentities does not include protamine.
133. The composition of any one of claims 123-132, with the provision that the plurality of nanoentities does not include polyarginine.
134. The composition of any one of claims 123-133, wherein the plurality of nanoentities do not comprise more than one outer shell.
135. (Aspect 8): Composition according to any one of claims 123-134 for use as a medicament.
136. A method, comprising administering the composition of any one of claims 123-134 to a living organism.
137. (Aspect 9): A composition, comprising: a plurality of nanoentities comprising an inner portion surrounded by an outer shell, the outer shell comprising polysialic acid and a targeting moiety comprising a cell-penetrating peptide chemically linked to the polysialic acid.
138. The composition of claim 137, with the proviso that the plurality of nanoentities are not liposomes.
139. (Aspect 10): Composition according to any one of claim 137 or 138 for use as a medicament.
140. A method, comprising administering the composition of any one of claim 137 or 138 to a living organism.
141. (Aspect 11): A composition, comprising: a plurality of nanocapsules comprising an inner portion surrounded by an outer shell, the outer shell comprising polysialic acid and a targeting moiety chemically linked to the polysialic acid, wherein the targeting moiety comprises a peptide having a sequence Z¹X¹X²Z² and/or a sequence RGD and/or a sequence NGR, wherein Z¹ is R or K, Z² is R or K, and X¹ and X² are each an amino acid residue.
142. The composition of claim 141, with the proviso that the plurality of nanoentities are not liposomes.
143. (Aspect 12): Composition according to any one of claim 141 or 142 for use as a medicament.
144. A method, comprising administering the composition of any one of claim 141 or 142 to a living organism.
145. (Aspect 13): A composition, comprising: a plurality of entities, having a maximum average diameter of less than about 1 micrometer, the entities having a surface comprising polysialic acid and a targeting moiety, with the proviso that the entities are not liposomes.
146. The composition of claim 145, wherein at least some of the plurality of nanoentities are nanocapsules.
147. The composition of any one of claim 145 or 146, wherein at least some of the plurality of nanoentities are micelles.
148. The composition of any one of claims 145-147, wherein the targeting moiety comprises a cell-penetrating peptide.
149. The composition of any one of claims 145-148, with the proviso that the plurality of entities are not liposomes.
150. (Aspect 14): Composition according to any one of claims 145-149 for use as a medicament.
151. A method, comprising administering the composition of any one of claims 145-149 to a living organism.
152. (Aspect 15): A kit comprising the composition as described in any one of claims 1-84, 94-120, 123-134, 137, 138, 141, 142, or 145-149.
153. (Aspect 16): A composition, comprising: a plurality of nanoentities comprising an inner portion surrounded by an outer shell, the outer shell comprising hyaluronic acid, at least some of the nanoentities further comprising a monoclonal antibody
154. The composition of claim 153, wherein at least about 90 wt % of the outer shell comprises hyaluronic acid.
155. The composition of any one of claim 153 or 154, wherein at least some of the plurality of nanoentities are nanocapsules.
156. The composition of any one of claims 153-155, wherein the inner portion is nonaqueous.
157. The composition of any one of claims 153-156, wherein the monoclonal antibody is contained within the inner portion.
158. The composition of any one of claims 153-157, wherein the plurality of nanoentities have an average diameter of less than 1 micrometer.
159. The composition of any one of claims 153-158, wherein the nanoentity is a micelle.
160. The composition of any one of claims 153-159, wherein the plurality of nanoentities does not comprise more than one outer shell.
161. (Aspect 17): Composition according to any one of claims 153-160 for use as a medicament.
162. (Aspect 18): A composition, comprising: a plurality of nanoentities comprising an inner portion surrounded by an outer shell, the outer shell comprising PGA and/or PASP and a targeting moiety.
163. The composition of claim 162, wherein the targeting moiety is bonded to the PGA and/or PASP electrostatically.
164. The composition of any one of claim 162 or 163, wherein the targeting moiety is bonded to the PGA and/or PASP via a linker.
165. The composition of any one of claim 162 or 163, wherein the targeting moiety is bonded to the PGA and/or PASP via an aminoalkyl (C₁-C₄) maleimide linker, an aminoalkyl (C₁-C₄) methacrylamide linker, or directly through an amide group.
166. The composition of claim 165, wherein the targeting moiety is bonded to the PGA and/or PASP via an aminoethylmaleimide linker.
167. The composition of any one of claims 162-166, wherein the targeting moiety comprising a cell-penetrating peptide.
168. The composition of any one of claims 162-167, wherein the cell-penetrating peptide is chemically linked to the PGA and/or PASP.
169. The composition of any one of claims 162-168, wherein the targeting moiety comprises a CendR peptide.
170. The composition of any one of claims 162-169, wherein the targeting moiety comprises Lyp-1.
171. The composition of any one of claims 162-170, wherein the targeting moiety comprises tLyp-1.
172. The composition of any one of claims 162-171, wherein the targeting moiety comprises cLyp1.
173. The composition of any one of claims 162-172, wherein at least about 90 wt % of the outer shell comprises PGA and/or PASP.
174. The composition of any one of claims 162-173, wherein at least some of the plurality of nanoentities are nanocapsules.
175. The composition of any one of claims 162-174, wherein the inner portion is nonaqueous.
176. The composition of any one of claims 162-175, wherein the inner portion comprises a pharmaceutical agent.
177. The composition of claim 176, wherein the pharmaceutical agent is a monoclonal antibody.
178. The composition of any one of claims 162-177, wherein the plurality of nanoentities have an average diameter of less than 1 micrometer.
179. The composition of any one of claims 162-178, wherein the nanoentity is a micelle.
180. The composition of any one of claims 162-179, wherein the plurality of nanoentities does not comprise more than one outer shell.
181. The composition of any of claims 162-180, wherein at least some of the PGA and/or PASP is linked to a hydrophobic moiety.
182. (Aspect 19): Composition according to any one of claims 162-181 for use as a medicament.
183. (Aspect 20): A composition, comprising: a plurality of nanocapsules comprising an inner portion surrounded by an outer shell, the outer shell comprising PGA and/or PASP and a targeting moiety, wherein the targeting moiety comprises a peptide having a sequence Z¹X¹X²Z² and/or a sequence RGD and/or a sequence NGR, wherein Z¹ is R or K, Z² is R or K, and X¹ and X² are each an amino acid residue.
184. The composition of claim 183, wherein the targeting moiety is bonded to the PGA and/or PASP electrostatically.
185. The composition of any one of claim 183 or 184, wherein the targeting moiety is bonded to the PGA and/or PASP via a linker.
186. The composition of any one of claim 183 or 185, wherein the targeting moiety is bonded to the PGA and/or PASP via an aminoalkyl (C₁-C₄) maleimide linker, an aminoalkyl (C₁-C₄) methacrylamide linker, or directly through an amide group.
187. The composition of claim 186, wherein the targeting moiety is bonded to the PGA and/or PASP via an aminoethylmaleimide linker.
188. The composition of any one of claims 183-187, wherein at least about 90 wt % of the outer shell comprises PGA and/or PASP.
189. The composition of any one of claims 183-188, wherein at least some of the plurality of nanoentities are nanocapsules.
190. The composition of any one of claims 183-189, wherein the inner portion is nonaqueous.
191. The composition of any one of claims 183-190, wherein the inner portion comprises a pharmaceutical agent.
192. The composition of claim 191, wherein the pharmaceutical agent is a monoclonal antibody.
193. The composition of any one of claims 183-192, wherein the plurality of nanoentities have an average diameter of less than 1 micrometer.
194. The composition of any one of claims 183-193, wherein the nanoentity is a micelle.
195. The composition of any one of claims 183-194, wherein the plurality of nanoentities does not comprise more than one outer shell.
196. The composition of any one of claims 183-195, wherein the targeting moiety comprises an amino acid sequence CJ¹Z¹X¹X²Z², wherein J¹ is an amino acid sequence.
197. The composition of any one of claims 183-196, wherein the targeting moiety comprises an amino acid sequence J¹RGD, wherein J¹ is an amino acid sequence.
198. The composition of any one of claims 183-197, wherein the targeting moiety comprises an amino acid sequence J¹Z¹X¹X²Z²J², wherein each of J¹ and J² is independently an amino acid sequence.
199. The composition of any one of claims 183-198, wherein the targeting moiety comprises an amino acid sequence J¹RGDJ², wherein each of J¹ and J² is independently an amino acid sequence.
200. The composition of any one of claims 183-199, wherein the targeting moiety comprises an amino acid sequence CJ¹Z¹X¹X²Z²J², wherein each of J¹ and J² is independently an amino acid sequence.
201. (Aspect 21): Composition according to any one of claims 183-200 for use as a medicament.
202. (Aspect 22): A composition, comprising: a plurality of nanoentities comprising an inner portion surrounded by an outer shell, the outer shell comprising PGA and/or PASP, at least some of the nanoentities further comprising a monoclonal antibody contained within the inner portion.
203. The composition of claim 202, wherein at least about 90 wt % of the outer shell comprises PGA and/or PASP.
204. The composition of any one of claim 202 or 203, wherein at least some of the plurality of nanoentities are nanocapsules.
205. The composition of any one of claims 202-204, wherein the inner portion is nonaqueous.
206. The composition of any one of claims 202-205, wherein the plurality of nanoentities have an average diameter of less than 1 micrometer.
207. The composition of any one of claims 202-206, wherein the nanoentity is a micelle.
208. The composition of any one of claims 202-207, wherein the plurality of nanoentities does not comprise more than one outer shell.
209. (Aspect 23): Composition according to any one of claims 202-208 for use as a medicament.
210. (Aspect 24): A composition comprising: a plurality of nanoentities comprising an inner portion surrounded by an outer shell, the outer shell comprising hyaluronic acid linked to a hydrophobic moiety.
211. The composition of claim 210, wherein at least some of the nanoentities further comprise a monoclonal antibody contained within the inner portion.
212. The composition of any one of claim 210 or 211, wherein at least some of the nanoentities further comprise a pharmaceutical agent contained within the inner portion.
213. The composition of any one of claims 210-212, wherein at least some of the nanoentities further comprise a small molecule contained within the inner portion.
214. The composition of any one of claims 210-213, wherein the hydrophobic moiety is selected from an alkyl group, cycloalkanes, bile salts and derivatives, terpenoids, terpenes, terpene-derived moieties and lipophilic vitamins.
215. The composition of any one of claims 210-214, wherein the hydrophobic moiety comprises a straight-chain alkyl group.
216. The composition of any one of claims 210-215, wherein the hydrophobic moiety comprises a C₂-C₂₄ straight-chain alkyl group.
217. The composition of any one of claims 210-216, wherein the hydrophobic moiety comprises a straight-chain C₁₆ alkyl group.
218. The composition of any one of claims 210-217, wherein at least some of the plurality of nanoentities further comprises a targeting moiety.
219. The composition of claim 218, wherein the targeting moiety comprises Lyp-1.
220. The composition of any one of claim 218 or 219, wherein the targeting moiety comprises tLyp-1.
221. The composition of any one of claims 218-220, wherein the targeting moiety comprises cLyp1.
222. The composition of any one of claims 218-221, wherein the targeting moiety comprises a cell-penetrating peptide.
223. The composition of any one of claims 210-222, wherein at least about 90 wt % of the outer shell comprises hyaluronic acid.
224. The composition of any one of claims 210-223, wherein at least some of the plurality of nanoentities are nanocapsules.
225. The composition of any one of claims 210-224, wherein the inner portion is nonaqueous.
226. The composition of any one of claims 210-225, wherein the plurality of nanoentities have an average diameter of less than 1 micrometer.
227. The composition of any one of claims 210-226, wherein the nanoentity is a micelle.
228. The composition of any one of claims 210-227, wherein the plurality of nanoentities does not comprise more than one outer shell.
229. (Aspect 25): Composition according to any one of claims 210-228 for use as a medicament.
230. (Aspect 26): A composition, comprising: a plurality of nanoentities comprising an inner portion surrounded by an outer shell, the outer shell comprising a polymer selected from the group consisting of polyacids, polyesters, polyamides, or mixtures thereof, at least some of the nanoentities further containing a monoclonal antibody.
231. The composition of claim 230, wherein the monoclonal antibody is contained within the inner portion.
232. The composition of any one of claim 230 or 231, wherein at least about 90 wt % of the outer shell comprises polymer.
233. The composition of any one of claims 230-232, wherein the polymer comprises polysialic acid.
234. The composition of any one of claims 230-233, wherein the polymer comprises hyaluronic acid.
235. The composition of any one of claims 230-234, wherein the polymer comprises polyglutamic acid and/or PGA-PEG.
236. The composition of any one of claims 230-235, wherein the polymer comprises PASP and/or PASP-PEG.
237. The composition of any one of claims 230-236, wherein the polymer comprises polylactic-polyethyleneglycol (PLA-PEG).
238. The composition of any one of claims 230-237, wherein the polymer comprises poly(lactic-co-glycolic acid) and/or pegylated poly (lactic-co-glycolic acid).
239. The composition of any one of claims 230-238, wherein the polymer comprises polylactic acid and/or pegylated polylactic acid.
240. The composition of any one of claims 230-239, wherein the polymer comprises polyasparaginic acid and/or pegylated polyasparaginic acid.
241. The composition of any one of claims 230-240, wherein the polymer comprises alginic acid and/or pegylated alginic acid.
242. The composition of any one of claims 230-241, wherein the polymer comprises polymalic acid and/or pegylated polymalic acid.
243. The composition of any one of claims 230-242, wherein the polymer is linked to a hydrophobic moiety.
244. The composition of any one of claims 230-243, wherein at least some of the nanoentities further comprise a targeting moiety.
245. The composition of any one of claims 230-244, wherein at least some of the plurality of nanoentities are nanocapsules.
246. The composition of any one of claims 230-245, wherein the inner portion is nonaqueous.
247. The composition of any one of claims 230-246, wherein the plurality of nanoentities have an average diameter of less than 1 micrometer.
248. The composition of any one of claims 230-247, wherein the nanoentity is a micelle.
249. The composition of any one of claims 230-248, wherein the plurality of nanoentities does not comprise more than one outer shell.
250. (Aspect 27): Composition according to any one of claims 230-249 for use as a medicament.
251. (Aspect 28): A composition, comprising: a plurality of nanoentities comprising an inner portion surrounded by an outer shell, the outer shell comprising hyaluronic acid linked to a hydrophobic moiety, at least some of the nanoentities further comprising a small molecule have a molecular weight of less than 1000 Da.
252. The composition of claim 251, wherein the small molecule is contained within the inner portion.
253. The composition of any one of claim 251 or 252, wherein the small molecule is a pharmaceutical agent.
254. The composition of any one of claims 251-153, wherein the small molecule is docetaxel.
255. The composition of any one of claims 251-254, wherein the hydrophobic moiety is selected from an alkyl group, cycloalkanes, bile salts and derivatives, terpenoids, terpenes, terpene-derived moieties and lipophilic vitamins.
256. The composition of any one of claims 251-255, wherein the hydrophobic moiety comprises a straight-chain alkyl group.
257. The composition of any one of claims 251-256, wherein the hydrophobic moiety comprises a C₂-C₂₄ straight-chain alkyl group.
258. The composition of any one of claims 251-257, wherein the hydrophobic moiety comprises a straight-chain C₁₆ alkyl group.
259. The composition of any one of claims 251-258, wherein at least about 90 wt % of the outer shell comprises hyaluronic acid.
260. The composition of any one of claims 251-259, wherein at least some of the plurality of nanoentities are nanocapsules.
261. The composition of any one of claims 251-260, wherein at least some of the nanoentities further comprise a targeting moiety.
262. The composition of any one of claims 251-261, wherein the targeting moiety comprises Lyp-1.
263. The composition of any one of claims 251-262, wherein the targeting moiety comprises tLyp-1.
264. The composition of any one of claims 251-263, wherein the targeting moiety comprises cLyp1.
265. The composition of any one of claims 251-264, wherein the targeting moiety comprises a cell-penetrating peptide.
266. The composition of any one of claims 251-265, wherein the targeting moiety is bonded to the hyaluronic acid.
267. The composition of any one of claims 251-266, wherein the inner portion is nonaqueous.
268. The composition of any one of claims 251-267, wherein the plurality of nanoentities have an average diameter of less than 1 micrometer.
269. The composition of any one of claims 251-268, wherein the nanoentity is a micelle.
270. The composition of any one of claims 251-269, wherein the plurality of nanoentities does not comprise more than one outer shell.
271. (Aspect 29): Composition according to any one of claims 251-270 for use as a medicament.
272. (Aspect 30): A kit comprising the composition as described in any one of claims 153-160, 162-181, 183-200, 202-208, 210-2298, 230-249, or 251-270.

The following examples are intended to illustrate certain embodiments of the present invention, but do not exemplify the full scope of the invention.

### Example 1

This example illustrates polysialic acid (PSA) nanocapsules functionalized or not functionalized with the tumor penetrating peptide tLyp1.

The composition of the nanocapsules was as follows. The nanocapsules were formed of an oily core surrounded by a polymer shell of PSA or PSA functionalized with tLyp1 peptide and stabilized by surfactants. The nanocapsules were formed due to the interaction of PSA with a positively charged surfactant at the interphase of an oil-in-water emulsion. Unless otherwise stated, the PSA used was around 30 kDa molecular weight (26-30 kDa, Serum Institute of India).

The covalent linking of PSA used allows a selective covalent binding between thiol groups of the peptide tLyp1 and carboxylate groups of PSA. This synthetic approach used the heterobifuncional linker aminoethyl maleimide which allows, first, its incorporation through the amine group of the linker to carboxylate groups of PSA (using carbodiimide chemistry) and second, peptide binding through the addition of the thiol group of the peptide (cysteine residue) to the maleimide group of the linker (Michael type addition), following a 2-step process (FIG. 1). This strategy allowed the preservation of the biologically active groups of tLyp1 peptide. Furthermore, the substitution degree can be easily controlled.

Polymeric nanocapsules, for example, PSA nanocapsules, can be produced by a variety of techniques. The number of tLyp1 molecules on the surface of the nanocapsules could be modified according to the different molar ratios used for the chemical reaction (see Table 1 shows the feed molar ratio of carboxylic acid (COOH) of PSA:EDC:NHS:AEM). One of them is a solvent displacement technique, involving the mixing of a polar solvent in a water phase. Another technique is a self-emulsification technique, which does not require the use of organic solvents.

Polymeric nanocapsules, for example, PSA nanocapsules, could be functionalized with tLyp1. The number of tLyp1 molecules on the surface of the nanocapsules could be controlled. The tLyp1-functionalized nanocapsules that were formed had a size around 130 nm and a negative zeta potential of (−44 mV). The tLyp1-functionalized nanocapsules were found to be stable upon incubation in plasma at 37° C. Moreover, the tLyp1-functionalized nanocapsules could be loaded with any suitable hydrophobic drug and also with hydrosoluble molecules. In one experiment, the tLyp1-functionalized nanocapsules were loaded with docetaxel, e.g., docetaxel anhydrous (Mw 807.289 g/mol; Log P 2.6). In addition to tLyp1, other tissue penetrating peptides, such as CendR peptides (e.g., Lyp1 and iRGD), may be linked to the PSA chain.

PSA was modified with N-(2-aminoethyl) maleimide trifluoroacetate salt. Different molar ratios between carboxylic acid groups of PSA and the EDC, NHS, AEM and tLyp1 were tested (Table 1). For this purpose, PSA was dissolved in 0.1 M MES buffer at pH 6 at a final concentration of 2 mg/mL, and the corresponding amount of EDC, NHS, and AEM were also dissolved in 0.1 M MES buffer, added to PSA solution, and maintained under magnetic stirring for 4 h at room temperature. The maleimide functionalized PSA (PSA-Mal) was purified by dialysis (regenerated cellulose, SnakeSkin 7 KDa MWCO, Thermo Scientific), first against NaCl 50 mM, and then against MilliQ water. For the second reaction, PSA-Mal was dissolved in a solution of 0.1 M MES buffer and NaCl 50 mM at a final PSA concentration of 1 mg/mL. The peptide was added to this solution and the reaction mixture was maintained for 4 h under magnetic stirring at room temperature, and the final PSA-tLyp1 product was purified by dialysis as described previously, freeze-dried (Pilot Lyophilizer VirTis Genesis 25 ES), and stored at 4° C.

TABLE 1

| | COOH (PSA) | EDC | NHS | AEM | tLyp1 |
|---|---|---|---|---|---|
| Ratio 0 | 1 | 0.29 | 0.05 | 0.01 | 0.022 |
| Ratio 1 | 1 | 0.58 | 0.1 | 0.02 | 0.044 |
| Ratio 2 | 1 | 1.16 | 0.2 | 0.04 | 0.069 |
| Ratio 3 | 1 | 1.8 | 0.3 | 0.06 | 0.106 |
| Ratio 4 | 1 | 2.16 | 0.36 | 0.07 | 0.177 |
| Ratio 5 | 1 | 2.4 | 0.40 | 0.08 | 0.142 |
| Ratio 6 | 1 | 3 | 0.50 | 0.10 | 0.177 |
| Ratio 7 | 1 | 4.5 | 0.75 | 0.15 | 0.266 |
| Ratio 10 | 1 | 5.8 | 1 | 0.20 | 0.0103 |
| Ratio 20 | 1 | 11.6 | 2 | 0.40 | 0.0283 |
| Ratio 30 | 1 | 17.4 | 3 | 0.60 | 0.0298 |
| Ratio 40 | 1 | 23.2 | 4 | 0.80 | 0.0647 |
| Ratio 50 | 1 | 29 | 5 | 1 | 0.06984 |
| Ratio 60 | 1 | 34.8 | 6 | 1.2 | 0.0841 |

EDC: N-(3-dimethylaminopropy1)-N′-ethylcarbodiimide hydrochloride; NHS: N-hydroxysuccinimide; AEM: N-(2-aminoethyl) maleimide trifluoroacetate salt

PSA nanocapsules were prepared as follows. Nanocapsules with a polymer coating of PSA (e.g., with different Mw of 8 kDa, 26-30 kDa and 94 kDa) or PSA-tLyp1 of different ratios were prepared by a solvent displacement technique. The organic phase was composed of 4.75 mL of acetone and 0.25 mL of ethanol containing 0.75 mg/mL of lecithin (Epikuron 145V, Cargill), 0.15 mg/mL of Cetyl Trimethyl Ammonium Bromide (CTAB, Sigma-Aldrich), 2.96 mg/mL of Caprylic/capric triglycerides (Miglyol® 812, IOI Eleo GmbH), and 150 micrograms/mL of docetaxel (Hao Rui Enterprises Ltd.) in the case of docetaxel-loaded nanocapsules. The aqueous phase was composed of 10 mL of PSA or PSA-tLyp1 solution at 0.25 mg/mL. The organic phase was added dropwise into the aqueous phase under magnetic stirring, leading to the immediate formation of the nanodroplets and the deposition of the polymer around them. After nanocapsule formation organic solvents were removed by rotavaporation. Results are presented as mean+/−SD of 3 replicates (Table 2).

Nanocapsules with a polymer coating of PSA or PSA-tLyp1 of different ratios were prepared by using a Nanoassemblr® Benchtop microfluidics instrument (Precision Nanosystems) as follows. The aqueous phase was composed of 10 mL of PSA or PSA-tLyp1 solution at 0.25 mg/mL. The organic phase was composed of 1 mL of ethanol containing 3.75 mg of Lipoid S100 (Lipoid GmbH), 0.75 mg of benzethonium chloride (Spectrum Chemical), 15.3 mg of Labrafac lipophile WL 1349 (Gattefosse) and 0.75 mg of docetaxel anhydrous (Hao Rui Enterprises Limited) in the case of docetaxel-loaded nanocapsules. Briefly, both aqueous and organic phase were injected into each inlet of the NanoAssemblr cartridge at an adjustable flow rate, where microscopic features engineered into the channel control the mixing of the two streams rapidly and homogeneously to produce the nanocapsules. After nanocapsule formation ethanol was removed by rotaevaporation. Increase of the operating flow rate was directly related with a decrease in the nanocapsules size (Table 3, PSA NCs-A to C). Results are presented as mean+/−SD of 3 replicates (Table 3).

Isolation/concentration of the nanocapsules. The nanocapsules were isolated by ultracentrifugation (Optima™ L-90K Ultracentrifuge, Beckman Coulter; Fullerton, Calif.) at 84035 g for 0.5 h at 15° C. Then infranatant was removed from the media. The nanocapsules (supernatant) were collected and diluted up to a known concentration.

Physico-chemical characterization of the nanocapsules. The nanocapsules were characterized in terms of mean particle size and polydispersity index (PI) by photon correlation spectroscopy (PCS). Samples were diluted in MilliQ Water and the analysis was carried out at 25° C. with an angle detection of 173°. Zeta potential measurements were performed by laser Doppler anemometry (LDA) and the samples were diluted in ultrapure MilliQ water. PCS and LDA analysis were performed in triplicate using a NanoZS® (Malvern Instruments, Malvern, UK).

Docetaxel association efficiency (AE %). The association efficiency of docetaxel was expressed as the percentage of drug encapsulated with respect to the total amount of docetaxel. Accordingly, the encapsulated drug was determined in an aliquot of isolated nanocapsules and the total amount of drug was estimated in an aliquot of non-isolated nanocapsules. The quantification of the drug was performed either by UPLC or by a liquid chromatography/tandem mass spectrometry method (LC-MS) using paclitaxel as the internal standard. The UPLC system included an Acquity UPLC® H-class system (Waters Corp) and a column compartment (BEH C18 column 2.1×100 mm, 1.7 micrometer, Waters). The experimental analytical conditions were as follows: the mobile phase included of MilliQ water (A) and acetonitrile (B). An isocratic program 55% A and 45% B was used. The flow rate was 0.4 mL/min, and the run time was 3.5 min. The temperature of the column was maintained at 40° C. and the autosampler was thermostatized at 4° C. The injected volume was 10 microliters. Under these conditions, DCX was eluted at 1.8+/−0.02 min. The LC-MS system included a UPLC system (Acquity UPLC® H-class system, Waters Corp; column compartment BEH C18 column 2.1×100 mm, 1.7 micrometers, Waters) coupled to a Xevo® Triple Quadrupole Detector (TQD) (Waters Corp, Milford, USA) with an electrospray ionization (ESI) interface. Mass spectrometric detection was operated in positive mode and set up for multiple reaction monitoring (MRM) to monitor the transitions of m/z 830.4 to 304.1 and 830.4 to 549.2. A temperature of 525° C. was selected as source temperature and 150° C. as desolvation temperature, the capillary voltage was 3.1 kV and the cone voltage was 40 V. Nitrogen was used for desolvation and as cone gas at a flow rate of 600 L/h and 80 L/h respectively. Argon was used as the collision gas. The optimized collision energy was 30 eV. The experimental analytical conditions were as follows: the mobile phase included 0.1% formic acid aqueous solution (A) and acetonitrile (B). A linear gradient program was used, starting with a 80% to 20% mobile phase A from 0 to 5 min, followed by a return to 80% of A from 5 to 5.5 min, and keeping it constant up to 6 min to reach the initial conditions. The flow rate was 0.6 mL/min, the total run time was 6 min. The temperature of the column was maintained at 40° C. and the autosampler was thermo statized at 4° C. The injected volume was 10 microliters. Under these conditions, DCX was eluted at 4.11+/−0.02 min. Data acquisition and analysis were performed using TargetLynx v4.1 software (Waters Corp.).

TABLE 2

| | | | Z potential | |
|---|---|---|---|---|
| | Size (nm) | PI | (mV) | AE % |
| Low Mw (8 kDa) | 147 | 0.07 | −46 | 33.7 |
| PSA NCs* | | | | |
| PSA NCs | 153 +/− 4.2 | 0.07 | −29.7 +/− 3.6 | 33.7 +/− 1.9 |
| High Mw (94 kDa) | 162 | 0.11 | −55 | 33.4 |
| PSA NCs* | | | | |
| PSA-tLyp1 NCs ratio 1* | 125.0 | 0.12 | −57.2 | n.d. |
| PSA-tLyp1 NCs ratio 2 | 140.5 +/− 2.0 | 0.05 | −45.6 +/− 1.0 | 26.1 +/− 4.5 |
| PSA-tLyp1 NCs ratio 3 | 143.5 +/− 4.1 | 0.06 | −28.0 +/− 2.2 | 20.4 +/− 2.2 |

| | |
|---|---|
| PSA-tLyp1 NCs ratio 4 | Aggregation |
| PSA-tLyp1 NCs ratio 5 | Aggregation |
| PSA-tLyp1 NCs ratio 6 | Aggregation |
| PSA-tLyp1 NCs ratio 7 | Aggregation |

*n = 1; PSA: polysialic acid; PSA-tlyp1: polysialic acid functionalized with the tlyp1 peptide; NCs: nanocapsules

TABLE 3

| | Total flow | | | Zpotential | |
|---|---|---|---|---|---|
| | rate (mL/min) | Size (nm) | PI | (mV) | AE % |
| PSA NCs-A* | 4 | 170.2 | 0.15 | −61.5 | — |
| PSA NCs-B* | 8 | 114.6 | 0.146 | −59 | — |
| PSA NCs-C* | 18 | 76.25 | 0.09 | −44.8 | — |
| PSA NCs-D | 7.5 | 118.7 +/− 2.1 | 0.15 | −52.3 +/− 3.7 | 36.5 +/− 1.4 |
| PSA-tLyp1 ratio 0 NCs | 7.5 | 136.0 +/− 4.6 | 0.10 | −49.8 +/− 4.8 | 34.6 +/− 4.2 |
| PSA-tLyp1 ratio 2 NCs | 7.5 | 124.3 +/− 2.5 | 0.12 | −51.0 +/− 1.5 | 34.4 +/− 2.4 |
| PSA-tLyp1 ratio 3 NCs | 7.5 | 135.3 +/− 5.0 | 0.13 | −41.5 +/− 4.1 | 31.8 +/− 2.5 |
| PSA-tLyp1 ratio 10 NCs | 6 | 146.7 +/− 10.7 | 0.13 | −44.5 +/− 2.3 | 30.1* |
| PSA-tLyp1ratio 30 NCs | 6 | 146.3 +/− 8.1 | 0.12 | −43.1 +/− 2.4 | 39.8* |
| PSA-tLyp1 ratio 40 NCs | 6 | 148.0 +/− 15.8 | 0.11 | −38.7 +/− 6.7 | 37.9* |
| PSA-tLyp1 ratio 60 NCs | 6 | 152 +/− 14.7 | 0.12 | −36.8 +/− 8.2 | 35.0* |

*n = 1; PSA: polysialic acid; PSA-tLyp1: polysialic acid functionalized with the tLyp1 peptide; NCs: nanocapsules.

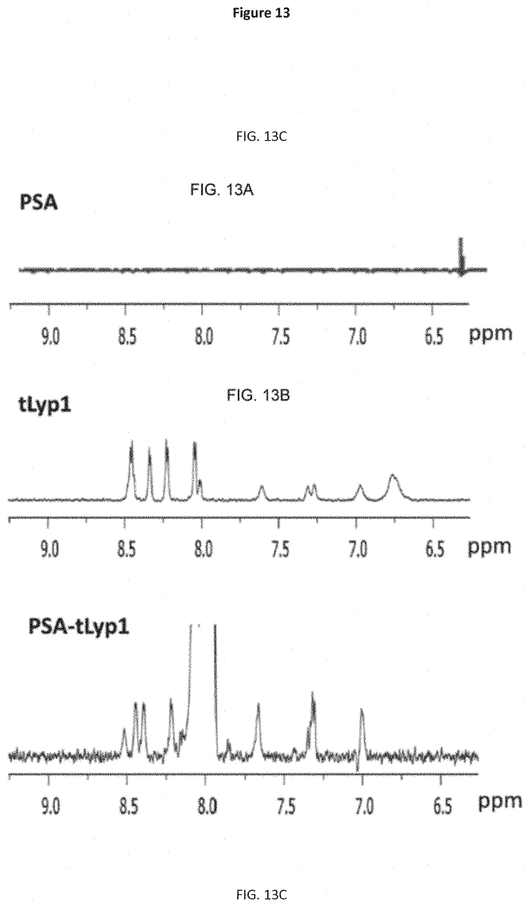

Characterization of the PSA-tlyp1 conjugate. Some NMR experiments were acquired on Varian Inova 750 spectrometers. The chemical shifts are reported in ppm. The spectra were recorded in a mixture of deuterium oxide: MilliQ water 10:90 at a polymer concentration between 0.4-0.8 mg/mL. ¹H-NMR analysis was performed at 750 MHz with 256 scans and 10 s of delay between each scan. MestreNova Software (Mestrelab Research) was used for spectral processing. The formation of the PSA-tLyp1 conjugate was confirmed by verifying the presence of characteristic ¹H-NMR signals from the peptide in PSA-tLyp1 spectra. Moreover, the presence of characteristic signals of amine protons from the amino acids of tLyp1 peptide were observed in the ¹H-NMR spectrum of PSA-tLyp1 between 6.5 and 8.5 ppm (FIG. 13), thus confirming the covalent linking between PSA and tLyp1.

### Example 2

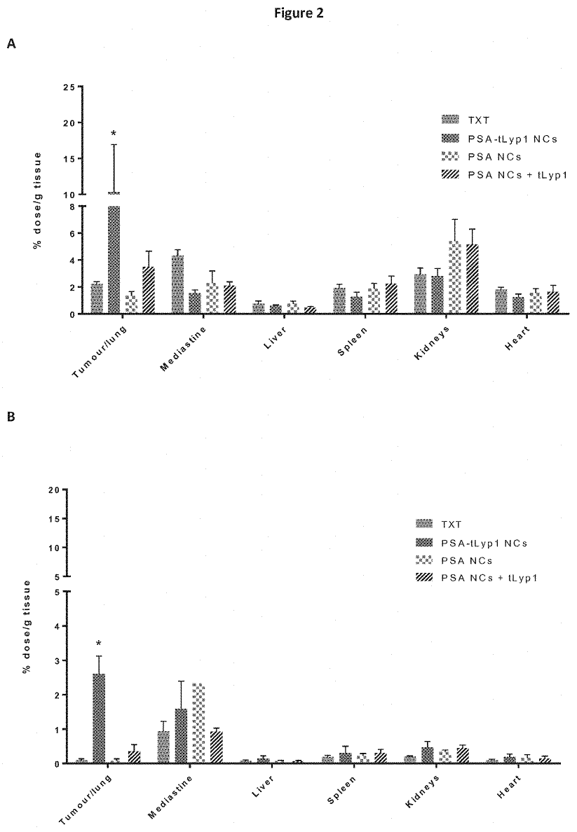

This example illustrates in vivo data using particles as described in Example 1. The functionalization of PSA with tLyp1 has resulted in a positive targeting effect in an orthotopic lung tumor model (high accumulation of the anti-tumor drug docetaxel in the lung). The biodistribution data presented in FIG. 2A indicate that this targeting effect is markedly pronounced when the peptide is attached (e.g., covalently linked) to PSA compared with the administration of unbound tLyp1 (PSA nanocapsules and tLyp1, e.g., separately) and non-modified PSA nanocapsules (without tLyp1). The biodistribution data presented in FIG. 2B indicate that the amount of docetaxel accumulated in the tumor (lung) after 24 h for those functionalized nanocapsules (PSA-tLyp1 NCs) was around 26-fold higher than that obtained for the marketed docetaxel Taxotere®.

The quantification of docetaxel in tissue and plasma samples was performed using a liquid chromatography/tandem mass spectrometry method (LC-MS) as described in Example 1. Tissue samples were weighed and homogenized in 8 mL of PBS 0.01 M per g of tissue using a gentleMACS™ Dissociator (Miltenyi Biotec). Drug extraction was performed by protein precipitation methodology using acetonitrile. To do this, 900 microliters of acetonitrile containing 9 ng of the internal standard paclitaxel were added to 100 microliters of plasma or homogenized tissue sample. Then, this mixture was vortexed for 20 min, centrifuged at 20817 g for 5 min, and 800 microliters of the resulting supernatant were collected and dried by evaporation (MiVac Duo Concentrator, Genevac) at 40° C. Finally, the resulting dried samples were dissolved in 100 microliters of mobile phase, filtered through 0.22 micrometers pore size (Millex-GV 4 mm, Millipore), and transferred to a LC vial. Calibration standards were generated in the same way by spiking blank plasma or tissues with docetaxel standard solutions. Under these conditions, the internal standard paclitaxel was eluted at 4.17+/−0.01 min, and the transitions 854.6 to 286 and 854.6 to 569 monitored. Data acquisition and analysis were performed using TargetLynx v4.1 software (Waters Corp).

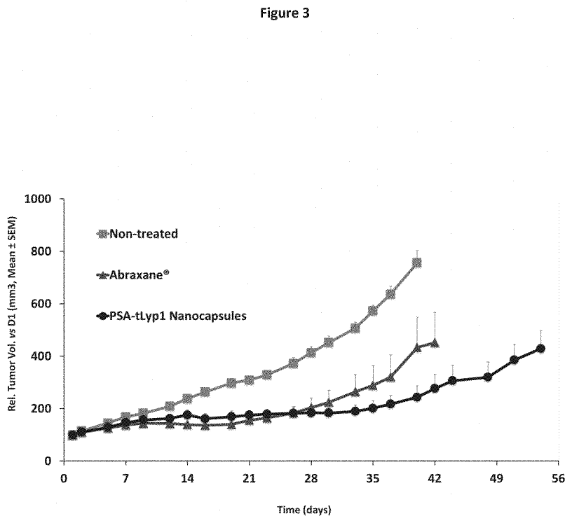

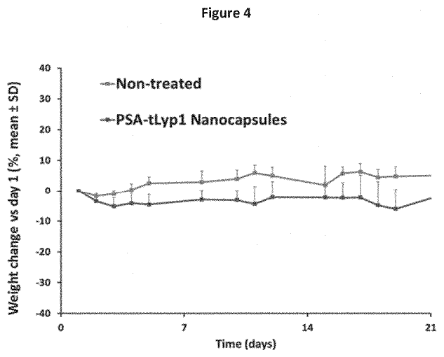

In this example, the efficacy of tLyp1 functionalized PSA nanocapsules was compared to that of the commercial formulation Abraxane® (paclitaxel) in a PDX (Patient Derived Xenograph) pancreatic cancer mice model. The results in FIG. 3 show tLyp1 functionalized PSA nanocapsules (Ratio 2) were more efficacious than Abraxane®. The growth of the tumor was significantly reduced and the survival of mice was significantly prolonged (42 vs. 56 days). Moreover, the nanocapsules showed low in vivo toxicity in terms of weight loss in healthy mice (FIG. 4) and blood toxicity.

FIG. 2 shows docetaxel accumulation at 1 h (FIG. 2A) and 24 h (FIG. 2B) after IV administration of Taxotere® (marketed docetaxel), PSA and PSA-tLyp1 NCs and tLyp1+PSA NCs, at an equivalent docetaxel dose of 7.5 mg/kg. Data are shown as mean+/−standard deviation (SD) of 5 replicates. Significant differences between the treatments (*) p<0.01.

FIG. 3 shows the relative tumor volume after IV administration of Abraxane® (paclitaxel dose of 150 mg/Kg) and docetaxel-loaded tLyp1-PSA nanocapsules (docetaxel dose of 60 mg/kg). All the data are given as mean+/−standard error (SEM) of 5 replicates. Mice died or were sacrificed at day 42 (control and treated with Abraxane®) or day 56 because of the advanced disease status.

FIG. 4 shows the evolution of body weight of mice treated with tLyp1-PSA nanocapsules at an equivalent total docetaxel dose of 75 mg/kg. All the data are given as mean+/−standard deviation (SD) of 5 replicates.

### Example 3

In addition to tLyp1, other targeting and/or tissue penetrating peptides, such as CendR peptides (e.g., cLyp1 and iRGD), may be linked to the polymeric chain, for example, to PSA.

cLyp1 was covalently linked to PSA using a similar chemical strategy to that used for PSA-tLyp1. First, PSA was modified with N-(2-aminoethyl) maleimide trifluoroacetate salt. Different molar ratios between carboxylic acid groups of PSA and the EDC, NHS, AEM and peptide (cLyp1) were tested (Table 4). For this purpose, PSA was dissolved in 0.1 M MES buffer at pH 6 at a final concentration of 2 mg/mL, and the corresponding amount of EDC, NHS, and AEM were also dissolved in 0.1 M MES buffer, added to PSA solution, and maintained under magnetic stirring for 4 h at room temperature. The maleimide functionalized PSA (PSA-Mal) was purified by dialysis as described in Example 1, first against NaCl 50 mM, and then against MilliQ water. In a second step, PSA-Mal was dissolved in a solution of 0.1 M MES buffer and NaCl 50 mM at a PSA concentration of 1 mg/mL. A linear form of the peptide with acetamidomethyl protecting groups in the cysteines 2 and 10 and without protective group in the cysteine 1, (H—CC(Acm)GNKRTRGC(Acm)-OH), was added to this solution and the reaction mixture was maintained for 24 h under magnetic stirring at room temperature. The PSA modified with the protected lineal form of the peptide was purified by dialysis with the same previous mentioned conditions. In order to obtain the final cyclic form of the peptide, deprotection of cysteine 2 and 10 of the peptide was carried out by adding 1 mL of HCl 1 M to PSA-peptide solution, second, cysteine oxidation reaction was performed adding a methanol solution of iodine (Sigma-Aldrich) containing 1 molar equivalent of I₂ respect to the peptide (5.10⁻³ M in methanol) over the conjugate under magnetic stirring for 1 h, then a drop of ascorbic acid (Panreac) 1M in water was added to this solution for neutralizing a possible I₂ excess from the medium. The final PSA-cLyp1 product was purified by dialysis, freeze-dried, and stored at 4° C. as previously described in Example 1.

The characterization of the PSA-cLyp1 conjugate was performed by ¹H-NMR.

TABLE 4
Feed molar ratio

| Ratio | PSA monomer | EDC | NHS | AEM | cLyp1 |
|---|---|---|---|---|---|
| 4 | 1 | 2.32 | 0.4 | 0.08 | 0.00066 |
| 5 | 1 | 2.9 | 0.5 | 0.10 | 0.0027 |
| 7.5 | 1 | 4.35 | 0.75 | 0.15 | 0.0066 |
| 10 | 1 | 5.8 | 1.0 | 0.20 | 0.0099 |
| 20 | 1 | 11.6 | 2.0 | 0.40 | 0.0241 |

Preparation of nanocapsules with PSA-cLyp1 and PSA-tLyp1 by a self-emulsifying technique. Briefly, 1.75 mL of an aqueous phase containing 5.95 mg of PSA-cLyp1 was added over an organic phase under magnetic stirring containing 118 mg of Labrafac lipophile WL1349 (Gattefosse), 116 mg of Polysorbate 80 (Tween 80, Merck), 5 mg of Macrogol 15 Hydroxystearate (Kolliphor HS15®, BASF), 0.4 mg of benzethonium chloride (Spectrum Chemical), 2 mg of Docetaxel anhydrous (Hao Rui Enterprises Limited), and 50 microliters of ethanol.

The nanocapsules were characterized in terms of mean particle size, polydispersity index (PI), and Zeta potential according to the methods described above Example 1. Total docetaxel content was estimated in an aliquot of non-isolated nanocapsules. The quantification of the drug was performed by UPLC according to the method previously described in Example 1. Results are presented as mean+/−SD of 3 replicates (Table 5).

TABLE 5

| | | | Zpotential | Total |
|---|---|---|---|---|
| | Size (nm) | PI | (mV) | docetaxel % |
| PSA-cLyp1 | 172.4 +/− 14.7 | 0.3 | 3.8 +/− 1.6 | 110.9 +/− 11.8 |
| (Ratio 20) NCs | | | | |
| PSA-tLyp1 | 117.2 +/− 14.7 | 0.3 | −0.1 +/− 2.9 | 118.7 +/− 21.6 |
| (Ratio 30) NCs | | | | |

PSA: polysialic acid;
PSA-tLyp-1: polysialic acid functionalized with the tLyp1 peptide;
NCs: nanocapsules;
PI: polydispersity index

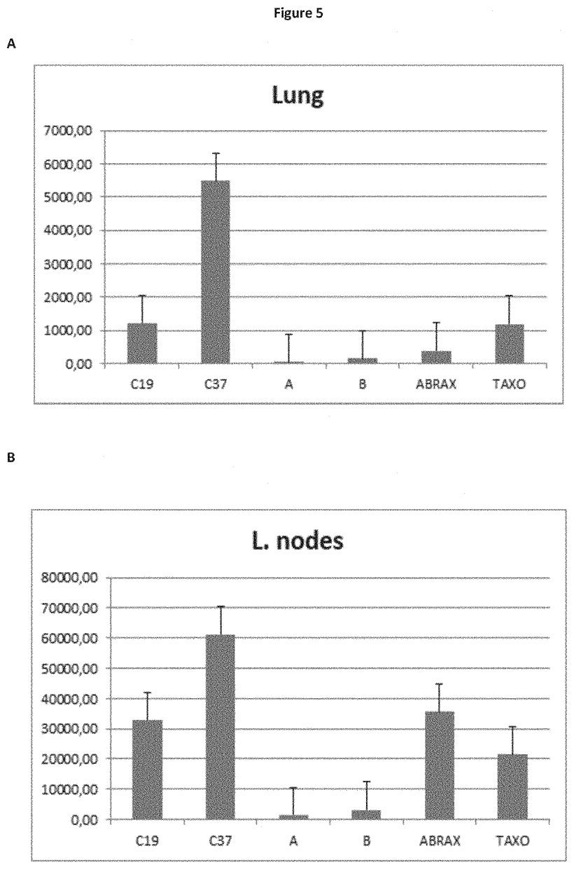

Preliminary in vivo efficacy studies. The efficacy of cLyp1 and tLyp1 functionalized PSA nanocapsules (5 mg/kg docetaxel) was compared to that of the commercial formulations Abraxane® (paclitaxel, 15 mg/kg) and Taxotere® (docetaxel, 5 mg/kg) in a metastatic orthotopic lung cancer model (A549 cells) in mice (n=3-4 animals/group). Quantification of luciferase activity ex vivo is depicted in FIG. 5: (i) in lungs (FIG. 5A), and (ii) in mediastinal lymph nodes (FIG. 5B) after the different treatments (TAXO, taxotere; ABRAX, Abraxane®; A, PSA-tLyp1 nanocapsules ratio 30; B, PSA-cLyp1 Ratio 20; C19, non-treated control at day 19; C37, non-treated control at day 37).

The results in FIG. 5 show a similar response in terms of reduction of tumor cells in the lung and in the lymph nodes of the mediastinum (metastasis) for both functionalized formulations. Interestingly, the functionalized nanocapsules were more efficacious than Abraxane® and Taxotere® in eliminating the metastasis. Moreover, no sign of toxicity was found for the nanocapsules in the analysis of weight loss, hemograms, and histopathology of vital organs (data not shown).

### Example 4

This example illustrates the possibility of increasing the batch production of nanocapsules and establish a scalable technology. Thus, for example, larger batches of PSA nanocapsules were prepared by solvent-displacement at a 10× scale (110 mL-batches).

The organic phase included 10 mL of ethanol containing 37.5 mg of phosphatidylcholine (Lipoid S100, Lipoid GmbH), 7.5 mg of benzethonium chloride (Spectrum Chemical), 152.8 mg of Caprylic/capric triglycerides (Labrafac Lipophile WL 1349, Gattefosse) and 7.5 mg of docetaxel anhydrous (Hao Rui Enterprises Limited). The aqueous phase was composed of 100 mL of a PSA solution at 0.25 mg/mL. The aqueous phase was maintained under an overhead propeller stirrer (Ika RW 20 digital) using a 4-bladded propeller (10M/M-P15) at 700 rpm and the organic phase was pumped into the aqueous phase throughout a peristaltic pump tubing (1.6×4.8×1.6 platinum-cured silicone, Freudemberg) using a peristaltic pump (Minipuls 3, Gilson) at 25 rpm. After nanocapsule formation organic solvents were removed by rotavaporation.

The nanocapsules were characterized in terms of mean particle size, polydispersity index (PI), and Zeta potential after isolation/concentration by ultracentrifugation according to the methods described above. The quantification of the drug was performed by UPLC according to the method previously described for AE % in Example 1. Results corresponding to 3 independent replicates are shown in Table 6.

TABLE 6

| | | | Zpotential | |
|---|---|---|---|---|
| | Size (nm) | PI | (mV) | AE % |
| PSA NCs | 160.9 +/− 7.0 | 0.1 | −52.7 +/− 2.1 | 44.7 +/− 14.5 |
| PSA-tLyp1 NCs R10 | 153.3 +/− 7.4 | 0.1 | −46.3 +/− 3.2 | 28.4 +/− 5.0 |
| PSA-tLyp1 NCs R20 | 156.0 +/− 3.6 | 0.05 | −42.7 +/− 5.0 | 46.5 +/− 3.6 |

PSA: polysialic acid;
PSA-tLyp-1: polysialic acid functionalized with the tLyp1 peptide;
NCs: nanocapsules;
R10: ratio 10;
R20: ratio 20;
PI: Polydispersity index

Moreover, isolation/concentration by tangencial flow filtration was evaluated as an alternative method to ultracentrifugation. Tangencial flow filtration is a scalable method which ideally allows to eliminate the rotavaporation step. Therefore, cross flow trials were conducted with a Sartoflow Smart Crossflow System (Sartorius). In these trials, a volume of 1 L of docetaxel-containing PSA nanocapsules (pool of 10 individual batches of 110 mL, no rotavaporated) was successfully concentrated at least 20× with the cassette Hydrosart 100 kDa. The average flow rate (LMH) was 120.6 L/hm². Results of 3 replicates in terms of isolation time, concentration factor, final docetaxel concentration and docetaxel association efficiency (indirect AE %, measured in the filtrate) are presented in Table 7.

TABLE 7

| | | Concentration | Final Docetaxel | AE (%) |
|---|---|---|---|---|
| | Isolation time | factor | concentration (ppm) | (indirect) |
| n1 | 22 min | 19.4-fold | 433 | 49.1 |
| n2 | 24 min | 23.9-fold | 540 | 48.7 |
| n3 | 25 min | 24-fold | 573 | 51.4 |

| | | |
|---|---|---|
| Average: | 515 +/− 73 | 50 +/− 1.5 |

Increasing of the batch production was also evaluated by using a self-emulsifying technique (100 mL batch size). For this, an aqueous phase containing 297.5 g of polymer and 87.5 g of water was added over an organic phase included 5900 mg of Labrafac Lipophile WL1349 (Gattefosse), 5800 mg of Polysorbate 80 (Tween 80, Merck), 250 mg of Macrogol 15 Hydroxystearate (Kolliphor HS15®, BASF), 20 mg of benzethonium chloride (Spectrum Chemical), 100 mg of Docetaxel anhydrous (Hao Rui Enterprises Limited) and 500 uL of ethanol, under an overhead propeller stirrer (IKA RW 20 digital) using a 4-bladded propeller (10M/M-P15) at 1000 rpm.

The nanocapsules were characterized in terms of mean particle size, polydispersity index (PI), and Zeta potential according to the methods described above in Example 1. Total docetaxel content/concentration was estimated in an aliquot of non-isolated nanocapsules. The quantification of the drug was performed by UPLC according to the method previously described in Example 1. Results corresponding to 3 replicates (PSA nanocapsules) and 1 replicate (PSA-tLyp1 NCs Ratio 10 and Ratio 20) are shown in Table 8.

TABLE 8

| | | | Zpotential | Total |
|---|---|---|---|---|
| | Size (nm) | PI | (mV) | docetaxel % |
| PSA NCs | 117.8 +/− 5.9 | 0.24 | −3.53 +/− 1.0 | 101.2 +/− 5.8 |
| PSA-tLyp1 NCs | 130.3 | 0.25 | −5.59 | 106.8 |
| Ratio 10* | | | | |
| PSA-tLyp1 NCs | 125.1 | 0.23 | −4.58 | 111.6 |
| Ratio 20* | | | | |

*n = 1;
PSA: polysialic acid;
PSA-tLyp-1: polysialic acid functionalized with the tLyp1 peptide;
NCs: nanocapsules;
PI: polydispersity index

A preliminary test for isolation/concentration of one pool of 10 batches of 100 mL by tangencial flow filtration (Sartoflow Smart Crossflow System, Sartorius) was performed according to the previously described conditions. Total docetaxel content/concentration was estimated in an aliquot of isolated nanocapsules (retentate), whereas AE % was determined in two different ways: (i) directly (retentate analysis) and (ii) indirectly (filtrate analysis). The quantification of the drug was performed by UPLC according to the method previously described in the Example 1. Results are shown in Table 9.

TABLE 9

| | Isolation | Concentration | Final Docetaxel | AE % | AE (%) |
|---|---|---|---|---|---|
| | time | factor | concentration (ppm) | (direct) | (indirect) |
| n1 | 22 min | 2.7-fold | 2346 | 82 | 98.5 |

### Example 5

This example illustrates the formulation of PSA nanocapsules associating other liposoluble small molecules, for example the anticancer drugs paclitaxel (856.903 g/mol; Log P 3.2; Teva) and patupilone (507.686 g/mol; Log P 3.7; Sigma-Aldrich).

Paclitaxel-loaded PSA nanocapsules were prepared by using a Nanoassemblr® Benchtop microfluidics instrument (Precision Nanosystems) as follows. The aqueous phase was composed of 10 mL of PSA solution at 0.25 mg/mL. The organic phase was composed of 1 mL of ethanol containing 3.75 mg of Lipoid S100 (Lipoid GmbH), 0.75 mg of Benzethonium chloride (Spectrum Chemical), 15.3 mg of Labrafac Lipophile WL 1349 (Gattefosse) and 0.75 mg of paclitaxel (Teva). Briefly, both aqueous and organic phase were injected into each inlet of the NanoAssemblr cartridge, at a total flow rate of 8 mL/min. After nanocapsule formation ethanol was removed by rotavaporation.

Patupilone-loaded PSA nanocapsules were also prepared by using a Nanoassemblr® Benchtop microfluidics instrument (Precision Nanosystems) using the conditions above, and just replacing paclitaxel by 0.75 mg of patupilone.

The nanocapsules were characterized in terms of mean particle size, polydispersity index (PI), and Zeta potential after isolation/concentration by ultracentrifugation according to the methods described above in Example 1. The quantification of the drug to determine AE % was performed by two different analytical methods, briefly:

(i) Paclitaxel: The quantification of the drug was performed by UPLC. The UPLC system included an Acquity UPLC® H-class system (Waters Corp) and a column compartment (BEH C18 column, 2.1×100 mm, 1.7 micrometers, Waters Corp.). The experimental analytical conditions were as follows: the mobile phase included MilliQ water (A) and acetonitrile (B). An isocratic program 55% A and 45% B was used. The flow rate was 0.6 mL/min, the run time was 4 min. The temperature of the column was maintained at 40° C. and the autosampler was thermostatized at 4° C. The injected volume was 10 microliters. Under these conditions, paclitaxel was eluted at 1.3 min.

(ii) Patupilone; the quantification of the drug was performed by HPLC. The HPLC system included a VWR Hitachi ELITE LaChrom (Hitachi) and a column compartment ACE Equivalence reversed-phase C18 (5 micrometers×250 mm×4.6 mm). The experimental analytical conditions were as follows: the mobile phase included MilliQ water acidified with 0.1% of formic acid (A) and acetonitrile (B). An isocratic program 80% A and 20% B was used. The flow rate was lml/min and the run time was 7.0 min. The temperature of the column was maintained at 30° C. The injected volume was 50 microliters. The detection wavelength was set at 248 nm. Under these conditions, patupilone was eluted at 3.55 min.

Results corresponding to 3 replicates of both paclitaxel and patupilone formulations are shown in Table 10.

TABLE 10

| | | | Zpotencial | |
|---|---|---|---|---|
| | Size (nm) | IP | (mV) | AE % |
| NCs PSA | 128.3 +/− 4.2 | 0.2 | −40.4 +/− 5.5 | 56.2 +/− 7.6 |
| (paclitaxel) | | | | |
| NCs PSA | 124.7 +/− 10.8 | 0.14 | −54.5 +/− 2.5 | 35.3 +/− 2.35 |
| (patupilone) | | | | |

### Example 6

One example for preparing C₁₂-functionalized PSA is as follows. C₁₂ (dodecyl) is used here, although other alkyl groups may similarly be used in other experiments. Referring to FIG. 5, a PSA sodium salt (30 kDa) was treated with Dowex and then tetrabutylammonium hydroxide. After concentration/purification by ultrafiltration and lyophilization of the concentrate, PSA tetrabutylammonium salt readily soluble in DMF was obtained.

The acid was then activated with 2-bromo-1-ethyl pyridinium tetrafluoroborate and subsequently reacted with dodecylamine. After isolation of the product by precipitation, tetrabutylammonium cation was replaced by a sodium cation. Concentration/purification by ultrafiltration and lyophilization of the concentrate gave target dodecylamide functionalized PSA sodium salt. Analysis by ¹H-NMR confirmed structure and degree of substitution in the range of 4%.

A few test reactions were performed to better optimize the amount of 2-bromo-1-ethyl pyridinium tetrafluoroborate. An initial test with 5% of 2-bromo-1-ethyl pyridinium tetrafluoroborate gave very low incorporation (<1%) of dodecylamine into the polymer. An experiment with 1 equivalent of 2-bromo-1-ethyl pyridinium tetrafluoroborate gave a product which was less soluble in water. With 30% of 2-bromo-1-ethyl pyridinium tetrafluoroborate, a degree of substitution in the range of 4% was obtained. The reaction was then scaled up to 1 gram of functionalized polymer.

Ultrafiltration. Ultrafiltration was used to concentrate (and desalt) the PSA tetrabutyl ammonium salt and the derivatized-PSA. Ultrafiltration was carried out using a Minim II Tangential Flow Filtration (TFF) System from Pall using a Cassette (Pall) 10K Omega centramate T-series 0.019 m² (part number OS010T02, serial number 36049076R, membrane lot number H5257E). Upon diafiltration, water was fed continuously into the reservoir and a permeate flow gets out. Salts and low molecular weight impurities permeated the membrane and were thus removed from the PSA solution.

PSA tetrabutyl ammonium salt (2). PSA sodium salt (1 g) was dissolved in pure water (100 mL) and was stirred 30 minutes with Dowex 50WX8 (200-400, H⁺ form; freshly washed with water followed by methanol and then by water) (20 mL) and the resin was filtered off and washed with deionized water. The pH of the solution was less than 4. The solution was treated with tetrabutylammonium hydroxide (40 wt % solution in water) until the pH was about 12. The whole procedure was repeated twice and the final pH was subsequently adjusted to 7.5-8 by bubbling CO₂ followed by bubbling N₂.

Ultrafiltration. The solution of PSA tetrabutyl ammonium salt was placed in the reservoir (400 mL) and the solution was concentrated to a volume of 100 mL. Upon diafiltration, water was fed continuously into the reservoir (300 mL). The permeate flow was 12 mL/min. When the diafiltration was finished, the solution was further concentrated to a minimum volume and removed from the reservoir. The transmembrane pressure during diafiltration was 0.6 bar, P₁=1.2 bar. The concentrate was lyophilized to give the title compound (1.6 g) as a white solid.

Dodecylamide functionalized PSA tetrabutyl ammonium salt (3). To a solution of PSA tetrabutyl ammonium salt 2 (1.3 g, 2.43 mmol eq.) in DMF (30 mL) under N₂ at room temperature was added 2-bromo-1-ethylpyridinium tetrafluoroborate (233 mg, 0.85 mmol, 0.35 eq.) in DMF (1 mL) and the solution was stirred for 1 h. A solution of 1-aminododecane (270 mg, 1.46 mmol) and Et₃N (0.576 mL, 4.13 mmol) in DMF (1 mL) was added to the reaction and the mixture was stirred for 40 h. The reaction mixture was added dropwise to a solution of Et₂O (150 mL) and acetone (15 mL). The precipitate was collected by filtration, washed with Et₂O and dried under reduced pressure.

Dodecylamide functionalized PSA sodium salt. The white precipitate was dissolved in deionized water (100 mL) and the solution was stirred 30 minutes with Dowex 50WX8 (200-400, H⁺ form; freshly washed with water followed by methanol and then water) (20 mL) and the resin was filtered off and washed with deionized water. The pH of the solution was less than 4. The solution was treated with aqueous sodium hydroxide (1 M) until the pH was 12. The whole procedure was repeated twice and the final pH was subsequently adjusted to 7.5-8 by bubbling CO₂ followed by bubbling N₂.

Ultrafiltration. The solution of derivatized-PSA sodium salt was placed in the reservoir (500 mL) and the solution was concentrated to a volume of 100 mL. Upon diafiltration water was fed continuously into the reservoir (500 mL). The permeate flow was 10.4 mL/min. When the diafiltration was finished, the solution was further concentrated to a minimum volume and removed from the reservoir. The transmembrane pressure during diafiltration was 0.6-0.7 bar (P₁=1.2-1.3 bar).

The concentrate was lyophilized to give the dodecylamide functionalized PSA sodium salt 4 (800 mg) as a white solid. ¹H-NMR indicated a degree of substitution around 4%.

### Example 7

Double functionalized polymers were prepared as follows, although other alkyl groups and targeting peptides may similarly be used in other experiments. For the preparation of C16-HA-tLyp-1, commercial C16-HA was used as starting material (Mw 55 kDa, substitution degree S.D. 7%, Contipro) and tLyp1 was chemically linked to the carboxylate groups of the HA backbone. Using a molar ratio respect to carboxilate groups from HA, EDC:NHS:AEM:tLyp1 of 1:2.16:0.36:0.072:0.0326 (Ratio 4). First, C16-HA was modified with N-(2-Aminoethyl) maleimide trifluoroacetate salt. For this purpose, C16-HA was dissolved in 0.1 M MES buffer at pH 6 at a final concentration of 2 mg/mL, and the corresponding amount of EDC, NHS, and AEM were also dissolved in 0.1 M MES buffer, added to C₁₋₆-HA solution, and maintained under magnetic stirring for 4 h at room temperature. The resulted product was purified by dialysis as described in Example 1 for PSA-tLyp1. In a second step, C16-HA-Mal was dissolved in a solution of 0.1 M MES buffer and NaCl 50 mM at a concentration of 1 mg/mL. Then tLyp1 was added to this solution and the reaction mixture was maintained for 24 h under magnetic stirring at room temperature. The final product was purified by dialysis and freeze-dried as described previously.

The characterization of this conjugate was performed by ¹H-NMR.

### Example 8

This example illustrates the formulation of different polymeric nanocapsules for the efficient association and delivery of monoclonal antibodies (mAbs). As non-limiting examples, the polymer-forming shells can be composed by biodegradable polyacids or polyamides, which can be further functionalized with targeting and/or tumor/tissue-penetrating ligands as, for example, tLyp-1. Nanocapsules with a polymer coating of PSA (8 kDa, 30 kDa or 94 kDa, Serum Institute of India), or PSA-tLyp1 Ratio 20, or C12-PSA (Example 6), or HA (330 kDa, Lehvoss Iberica), or C16-HA (different Mw and alkyl substitution degree: 55 kDa-S.D. 7%; 216 kDa-S.D 5%; 216 kDa-S.D. 11%, Contipro), or C16-HA-tLyp1 (Example 7), or polyglutamic acid (PGA, 11.9 KDa, Polypeptide Therapeutic Solutions), or PGA-PEG (PGA 6.68 KDa/PEG 5 KDa, Polypeptide Therapeutic Solutions), or the polyamide polyaspartic acid (PASP, Poly-L-aspartic acid, 200 units, average Mw 27 kDa, Alamanda Polymers), or PASP-PEG (Methoxy-poly(ethylene glycol)-block-poly(L-aspartic acid sodium salt, mPEG5K-b-PLD200, average MW 32 kDa, Alamanda Polymers) were prepared by a self-emulsifying technique.

Preparation of blank nanocapsules (without antibody). First, 59 mg Polysorbate 80 (Tween 80®, Merck) and 58 mg caprylic/capric triglycerides (Mygliol® 812N, IOI Oleochemical GmbH) were weighted in a glass vial of 2 mL capacity (oily phase). Then, for those formulations containing non-hydrophobically modified or non-amphiphilic polymers as shells, a cationic surfactant was added to the oily phase (4 microliters of benzethonium previously solubilized in ethanol, 50 mg/mL). All components of the oily phase were kept under magnetic stirring (500 rpm). In parallel, the aqueous phase was prepared by solubilizing, separately, each polymer in PBS pH 7.3 25 mM at variable concentrations (for example, for PSA-based formulations at 3 mg/mL, for HA-based formulations at 0.25 mg/mL, for PGA at 3 mg/mL, for PGA-PEG at 6 mg/mL, for PASP at 3 mg/mL and for PASP-PEG at 6 mg/mL), and Macrogol 15 Hydroxystearate (Kolliphor HS15®, BASF) also solubilized in PBS pH 7.3 25 mM at a concentration of 20 mg/mL. After that, 0.75 mL of the polymer solution were conveniently mixed with 125 microliters of the Kolliphor solution and this aqueous phase was added over the oily phase under magnetic stirring (1100 rpm).

Association of mAbs. Two different methods were used:

(i) 1-step method: The required volume of mAb in solution with the required concentration to get a desired final mAb concentration (in one instance 0.5 mg/mL), was added to the aqueous phase before being mixed with the oily phase. The mAbs associated to the nanocapsules by the 1-step method were anti-PD-L1 mAb (rat anti-mouse IgG2a, BioXcell®) and bevacizumab (humanized IgG1, Selleck Chemicals LLC).

(ii) 2-steps method: a solution containing the mAb at the desired concentration (in one instance 1 mg/mL) was added to the pre-formed nanocapsules under orbital stirring (550 rpm) to get a final mAb concentration of, for example, 0.5 mg/mL. The mAb was incubated with the nanocapsules during 4 hours at room temperature. A non-limiting example of mAbs associated to the nanocapsules by 2-steps method is the anti-PD-L1 mAb (rat anti-mouse IgG2a, BioXcell®) (Table 14).

The nanocapsules were characterized in terms of mean particle size, polydispersity index (PI), and Zeta potential according to the methods described above. Results corresponding to 3 replicates are shown in Table 13 (1-step method, anti-PD-L1), Table 14 (2-steps method, anti-PD-L1) and Table 16 (1-step method, bevacizumab).

Association efficiency of monoclonal antibodies. To determine the association of mAbs to the nanocapsules, a 1 mL aliquot of each different formulation was filtered in Amicon Stirred Cells® (polyethersulfone Biomax® 500 KDa Ultrafiltration Discs, Merck) at 4° C. under 1 bar nitrogen pressure. After this isolation process, the filtrate containing the free mAb was taken and analyzed by the corresponding ELISA assay. The association efficiency was indirectly calculated as: (total mAb−free mAb)/Total mAb*100. Results are showed in Table 11 (1-step method, anti-PD-L1), Table 14 (2-steps method, anti-PD-L1) and Table 16 (1-step method, bevacizumab).

Study of susceptibility to leakage upon dilution at room temperature (RT). To evaluate if mAbs were strongly entrapped in the nanoestructures, mAb association upon dilution (1:2->1:16) in PBS pH 7.3 (25 mM) at RT was evaluated according to the method described above for the mAb association efficiency. The results obtained for the different mAb association methods are shown in Table 12 and Table 16, for the 1-step formulations, and in Table 15 for the 2-steps formulations.

In vitro release study. mAb-loaded nanocapsules were incubated in PBS pH 7.3 (25 mM) at 37° C. (1:10 dilution). At predetermined times (1 h and 2 h), samples were taken and filtered according to the method described above to quantify by ELISA the released mAb (Table 13, 1-step method, anti-PD-L1).

Tables 11-16 demonstrate the possibility of formulating different polymeric nanocapsules with adequate physico-chemical properties and high association efficiencies for different mAbs. The association of mAbs can be done by, for example, the 1-step and 2-steps methods explained above, however, 1-step method provided a better entrapment of the mAb into the nanostructure, as it can be concluded from the study of susceptibility to mAb leakage upon dilution performed (Table 12 for 1 step; Table 15 for 2-steps).

TABLE 11
1-step method (Anti-PD-L1, 0.5 mg/mL)

| | | | Zeta potential | Association |
|---|---|---|---|---|
| Formulation | Size (nm) | PI | (mV) | efficiency (%) |
| PSA | 142 +/− 6 | 0.21 | −6 +/− 1 | 79 +/− 3 |
| PSA 94 KDa | 171 +/− 5 | 0.25 | −5 +/− 1 | 73 +/− 7 |
| C12-PSA | 138 +/− 8 | 0.29 | −3 +/− 1 | 75 +/− 3 |
| HA | 163 +/− 3 | 0.23 | −6 +/− 1 | 61 +/− 5 |
| [PGA]₅₀ | 167 +/− 7 | 0.23 | −6 +/− 1 | 69 +/− 10 |

TABLE 12

| | 1-step method (% Anti-PD-Ll (0.5 mg/mL) remaining entrapped) | | | | |
|---|---|---|---|---|---|
| Dilution | PSA | PSA 94 Kda | C12-PSA | HA | [PGA]₅₀ |
| 1:2 | 63 +/− 3 | 65 +/− 18 | 76 +/− 1 | 70 +/− 4 | 72 +/− 1 |
| 1:4 | 64 +/− 12 | 58 +/− 2 | 61 +/− 1 | — | 61 +/− 11 |
| 1:8 | 66 +/− 15 | 68 +/− 6 | 43 +/− 5 | 71 +/− 7 | 73 +/− 11 |
| 1:16 | 74 +/− 4 | 55 +/− 3 | 63 +/− 5 | — | 62 +/− 6 |

TABLE 13

| | 1-step method (Percent of mAb released upon 1:10 | | | | |
|---|---|---|---|---|---|
| | dilution at 37° C.) (Anti-PD-L1, 0.5 mg/mL) | | | | |
| Time | PSA | PSA 94 KDa | C12-PSA | HA | [PGA]₅₀ |
| 1 hour | 27 +/− 7 | 24 +/− 6 | 26 +/− 5 | 21 +/− 2 | 31 +/− 2 |
| 2 hours | 25 +/− 4 | 29 +/− 1 | 23 +/− 3 | 30 +/− 6 | 45 +/− 15 |

TABLE 14
2-steps method (Anti-PD-L1, 0.5 mg/mL)

| | | | Zeta potential | Association |
|---|---|---|---|---|
| Formulation | Size (nm) | PI | (mV) | efficiency (%) |
| PSA | 132 +/− 2 | 0.21 | −4 +/− 1 | 57 +/− 8 |
| PSA 94 KDa | 132 +/− 2 | 0.23 | −5 +/− 1 | 59 +/− 15 |
| C12-PSA | 113 +/− 2 | 0.22 | −9 +/− 1 | 67 +/− 5 |
| HA | 143 +/− 6 | 0.22 | −4 +/− 1 | 57 +/− 7 |
| [PGA]₅₀ | 178 +/− 3 | 0.18 | −5 +/− 1 | 59 +/− 1 |

TABLE 15

| | 2-steps method (% Anti-PD-L1 (0.5 mg/mL) | | | | |
|---|---|---|---|---|---|
| | remaining entrapped) | | | | |
| Dilution | PSA | PSA 94 Kda | C12-PSA | HA | [PGA]₅₀ |
| 1:2 | 27 +/− 11 | 20 +/− 8 | 61 +/− 8 | 27 +/− 10 | 53 +/− 8 |
| 1:4 | 31 +/− 3 | n.d | 33 +/− 2 | 28 +/− 13 | 16 +/− 8 |
| 1:8 | 2 +/− 8 | 0 +/− 17 | 4 +/− 1 | 6 +/− 7 | 0 +/− 2 |

TABLE 16
1-step method (Bevacizumab, 0.5 mg/mL)

| | | | | | % mAb |
|---|---|---|---|---|---|
| | | | | | remaining |
| | | | | Association | entrapped |
| | | | | efficiency | (1:16 |
| Formulation | Size | PDI | ZPotential | (%) | dilution) |
| PSA | 170 +/− 4 | 0.25 | −2.4 +/− 1 | 73 +/− 7 | 73 +/− 2 |
| PSA 94 kDa | 157 +/− 13 | 0.23 | −2.2 +/− 1 | 69 +/− 6 | 73 +/− 5 |
| C12-PSA | 127 +/− 6 | 0.26 | −2.6 +/− 3 | 69 +/− 8 | 70 +/− 5 |
| PSA-tLyp1 | 177 +/− 5 | 0.27 | −5 +/− 1 | — | — |
| Ratio 20 | | | | | |
| [PGA]₅₀ | 160 +/− 5 | 0.25 | −3.4 +/− 1 | 76 +/− 2 | 71 +/− 6 |
| [PGA]₁₀ | 151 +/− 9 | 0.25 | −0.3 +/− 2 | 74 +/− 5 | 75 +/− 5 |
| PEG_{2.5K} | | | | | |
| C16-HA 55 | 131 +/− 6 | 0.28 | −7 +/− 1 | 71 +/− 7 | 74 +/− 4 |
| DS 7% | | | | | |
| C16-HA- | 133 +/− 2 | 0.31 | −7 +/− 1 | — | — |
| tLyp1 | | | | | |
| Ratio 4 | | | | | |
| C16-HA 216 | 117 +/− 6 | 0.28 | −6 +/− 3 | 83 +/− 7 | 68 +/− 10 |
| DS 5% | | | | | |
| C16-HA 216 | 131 +/− 1 | 0.28 | −11.6 +/− 1 | 71 +/− 2 | 77 +/− 1 |
| DS 11% | | | | | |
| PASP (*) | 126 +/− 8 | 0.30 | −8 +/− 1 | — | 58 +/− 10 |
| PASP | 131 +/− 4 | 0.27 | −4 +/− 1 | — | 70 +/− 2 |
| PEG (*) | | | | | |

(*) 1 mg/mL bevacizumab

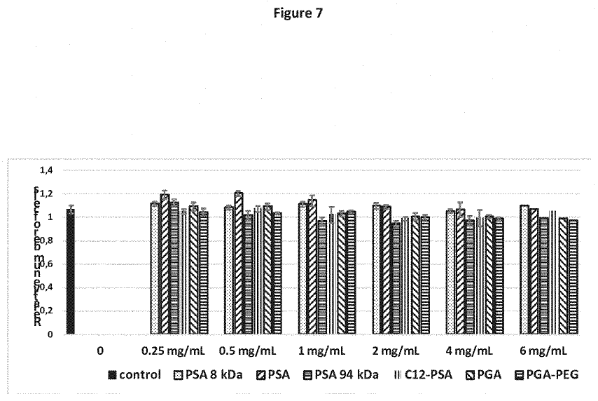

Cytotoxicity of different blank polymeric nanocapsules (without mAb). Cytotoxicity was determined using a crystal violet assay as an indicator of cell viability. Cell viability was assessed after the co-incubation of MDA-MB-231 cells seeded on a 96-well tissue cultured plate with the aforementioned formulations in dispersion (at different concentrations) in cell culture medium during 2 h. As can be observed in FIG. 7, the viability of the cells was higher than 80% in all cases at concentrations up to 6 mg/mL.

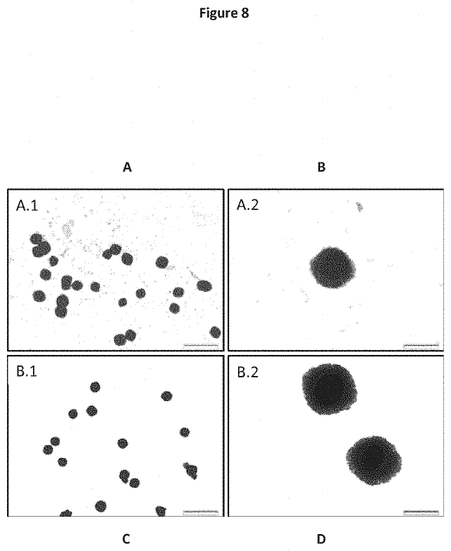

Morphological analysis of mAb-loaded polymeric nanocapsules. The morphological analysis of mAb (bevacizumab)-loaded nanocapsules was carried out with transmission electron microscopy (TEM, CM12, Philips, Netherlands). The samples were stained with phosphotungstic acid (2%, w/v) solution and placed on cupper grids with Formvard® for TEM observation. TEM photographs of PSA nanocapsules (A) and HA 216 SD 5% (B) containing Bevacizumab (final concentration of 3 mg/mL) are shown in FIG. 8 (1 micrometer size bar for FIGS. 8A and 8C, 200 nm size bar for FIGS. 8B and 8D).

Freeze-drying studies. Additionally, a freeze-drying study was performed to assess the possibility to process mAb-containing nanocapsules suspensions as powders for long-term storage. As non-limiting example, different bevacizumab-loaded polymeric nanocapsules were prepared by the 1-step method explained above, and a concentrated solution of trehalose and mannitol was added to the nanocapsules suspension (final concentration of trehalose 5% w/v and mannitol 2.5% w/v) prior to freeze-drying (˜50 hours cycle; Pilot Lyophilizer VirTis Genesys 25 ES). The stability of the freeze dried nanocapsules stored at 4° C. during 4 months was analyzed and compared with the initial values (before freeze-drying) by measuring particle size, PI, pH, Zeta potential and total mAb content (by ELISA). The measurements were done in the same way as described above. Results corresponding to 3 replicates are shown in Table 17, where it is shown that no significant changes were produced in terms of physico-chemical properties after 4 months under storage, whereas the total mAb percent was around 80-90%.

TABLE 17

| | Size (nm) | | pH | | PI | | ZPotential | | % mAb |
|---|---|---|---|---|---|---|---|---|---|
| | | 4 | | 4 | | 4 | | 4 | 4 |
| Formulation | Initial | months | Initial | months | Initial | months | Initial | months | months |
| PSA | 110 ± 7 | 115 ± 13 | 7.18 | 7.11 | 0.21 | 0.20 | −4 ± 1 | −4 ± 2 | 87 ± 2 |
| C12-PSA | 104 ± 9 | 104 ± 7 | 7.15 | 7.13 | 0.22 | 0.17 | −4 ± 1 | −8 ± 1 | 80 ± 10 |
| C16-HA 216 SD 5% | 117 ± 16 | 97 ± 3 | 7.16 | 7.10 | 0.27 | 0.20 | −9 ± 3 | −9 ± 1 | 78 ± 6 |

### Example 9

This example illustrates the formulation of alternative polymeric nanocapsules for the efficient association and delivery of monoclonal antibodies (mAbs). The polymer-forming shells can be composed by biodegradable water-insoluble polymers such as pegylated poly(lactic-co-glycolic acid) (PLGA-PEG or PLG-PEG) or pegylated polylactic acid (PLA-PEG), which can be further functionalized with targeting and/or tumor/tissue-penetrating ligands as, for example, tLyp-1.

Nanocapsules with a polymer coating of PLA-PEG were prepared by a solvent displacement method, associating the antibody by the 1-step method. Briefly, for the preparation of a 5 mL-batch:

(1) Preparation of the oily phase: 290 mg of Polysorbate 80 (Tween 80®, Merck) and 295 mg of Mygliol® 812N (IOI Oleochemical GmbH) were weighted in a glass vial of 25 mL mixed under magnetic stirring (500 rpm). The PLA-PEG polymer was solubilized in 12.5 mL of acetone, added to the previous solution and kept under magnetic stirring (500 rpm);

(2) Preparation of the aqueous phase: 625 microliters of a solution of Kolliphor HS15® (20 mg/mL in PBS pH 7.3 25 mM) were mixed with 4.4 mL of PBS pH 7.3 25 mM containing the corresponding amount of mAb and 25 mL of water in a glass vial of 100 mL capacity.

Then, the oily phase was added to the aqueous phase under magnetic stirring (1250 rpm) using a 20 mL-syringe (needle 120×40 mm), leading to the immediate formation of the nanodroplets and the deposition of the polymer around them. Final NCs suspension was rotavaporated until reach 5 mL. The nanocapsules were characterized in terms of mean particle size, polydispersity index (PI), Zeta potential and association upon 1:16 dilution, according to the methods described above. Results corresponding to 3 replicates are shown in Table 18.

TABLE 18

| | | | Zeta | Association (%) |
|---|---|---|---|---|
| Formulation | Size (nm) | PDI | potential | 1:16 dilution |
| PLA-PEG | 168 +/− 1 | 0.12 | −8 +/− 1 | 63 +/− 7 |
| (0.5 mg/mL Bevacizumab) | | | | |

### Example 10

One of the main limitations of nanocarriers is the limited drug association efficiency and loading capacity at clinically translatable doses. Thus, the influence of the antibody concentration on the physicochemical properties of, for example, PSA nanocapsules, and their mAb association efficiency and entrapment was evaluated for bevacizumab as mAb model (Selleck Chemicals LLC). The method used for mAb association was the 1-step procedure.

The nanocapsules were characterized in terms of mean particle size, polydispersity index (PI), Zeta potential, association efficiency and association upon 1:16 dilution, according to the methods described above (Example 8). Results corresponding to 3 replicates are shown in Table 19.

Final bevacizumab concentrations of at least 5 mg/mL were reached without significantly affecting nanocapsules properties and maintaining a high association efficiency of 70%, which represents a mAb loading content around 3% (mAb loading content=weight of mAb associated/total weight of nanocapsules components).

TABLE 19

| | | | | Association | Entrapment |
|---|---|---|---|---|---|
| mAb final | Size | | ZPotential | efficiency | % after 1:16 |
| concentration | (nm) | PI | (mV) | (%) | dilution |
| 0.5 mg/ml | 170 +/− 4 | 0.25 | −2.37 +/− 1 | 71.5 +/− 5.8 | 73 +/− 2 |
| 1 mg/ml | 179 +/− 14 | 0.28 | −0.52 +/− 2 | 59.8 +/− 0.2 | 75 +/− 1 |
| 3 mg/ml | 165 +/− 14 | 0.24 | −1.13 +/− 1 | 70.0 +/− 8 | 72 +/− 11 |
| 5 mg/ml | 172 +/− 14 | 0.26 | −0.40 +/− 2 | 70 +/− 9 | 78 +/− 8 |

### Example 11

Often, the lack of efficacy of nanocarriers is a result of their aggregation in complex media, and this may result from the high ionic strength and/or the presence of proteins in biological media. Thus, the stability in plasma of different mAb-loaded polymeric nanocapsules was explored, as an indicator of their potential for parenteral administration of mAbs.

Stability in plasma. Bevacizumab-loaded nanocapsules prepared by 1-step procedure as in the Example 8 were incubated in mouse plasma (dilution 1:10, 37° C.) under horizontal shaking (300 rpm, Heidolph Instruments GmbH & Co.). At predetermined times, samples of the incubation milieu were withdrawn for the analysis of particle size with Malvern Zeta-Sizer and for the analysis of size and size distribution by Nanoparticle Tracking Analysis (NTA). Samples were analyzed after an appropriate further dilution (1:10.000 in PBS pH 7.4 10 mM for NTA; 1:1000 in water for DLS).

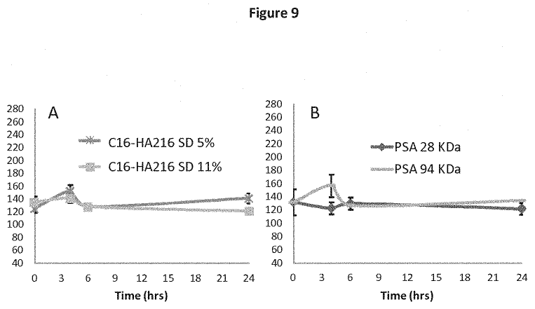

The stability of different mAb-loaded polymeric nanocapsules measured by DLS is represented in FIG. 9 (FIG. 9A: C16-HA based nanocapsules; FIG. 9B: PSA-based nanocapsules). Results represent the average of 3 replicates.

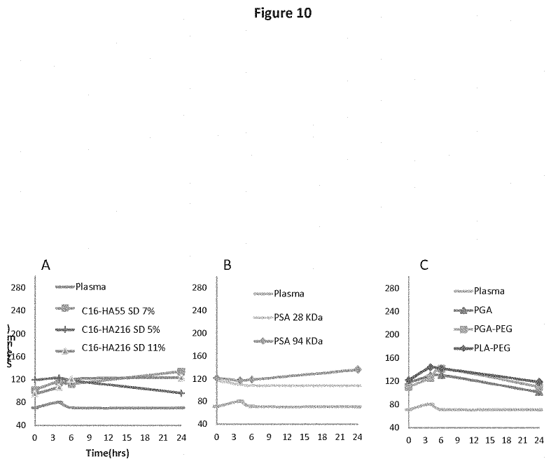

The stability of different mAb-loaded polymeric nanocapsules measured by NTA is represented in FIG. 10 (FIG. 10A: C16-HA based nanocapsules; FIG. 10B: PSA-based nanocapsules; FIG. 10C: PGA-based nanocapsules and PLA nanocapsules; n=1).

All the mAb-loaded nanocapsules showed an adequate stability in a complex media such as plasma during at least 24 h, which represents an important advantage to be parenterally administered to a subject.

### Example 12

This example illustrates the capacity of different polymeric nanocapsules to interact with cells and further elicit the cell internalization of the associated antibody in vitro. In order to perform this study, different nanocapsules associating a fluorescent antibody model (FITC-IgG, >98% purity, Elabsciences) were prepared by the 1-step method and characterized in terms of size, PI and zeta potential, as explained in Example 8 (Table 20).

TABLE 20

| | FITC-IgG concentration | | | | | | | | |
|---|---|---|---|---|---|---|---|---|---|
| | 1 mg/ml | | | 1.75 mg/ml | | | 2 mg/ml | | |
| | Size | | ZPotential | Size | | ZPotential | Size | | ZPotential |
| Formulations | (nm) | PI | (mV) | (nm) | PI | (mV) | (nm) | PI | (mV) |
| C16-HA216 | 131 ± 2 | 0.21 | −18 ± 1 | 144 ± 1 | 0.31 | −16 ± 4 | — | — | — |
| SD 5% | | | | | | | | | |
| PSA | 182 ± 1 | 0.26 | −10 ± 1 | 176 ± 2 | 0.32 | −11 ± 1 | 154 ± 3 | 0.23 | −13 ± 1 |
| PSA 94 kDa | — | — | — | — | — | — | 150 ± 3 | 0.24 | −11 ± 1 |
| PSA C12 | — | — | — | — | — | — | 138 ± 2 | 0.23 | −11 ± 1 |

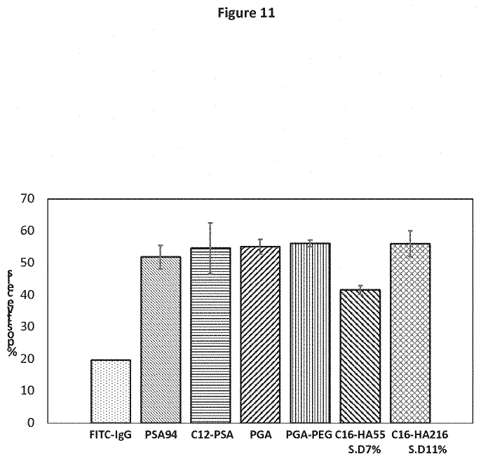

First, a flow cytometer study was performed to know the ability of the nanocapsules to interact with the cells. Different polymeric nanocapsules (diluted in cell culture medium at a final concentration of 7 mg/mL of nanocapsules and 105 micrograms/mL of IgG-FITC) were added to MBD-MB-231 cells in culture (66,500 cells/well) and left for 2 hours incubating at 37° C. (humidified incubator at 37° C. with 5% CO₂). After the incubation the cells were gently washed twice with PBS and then trypsinized to perform the Flow cytometry analysis. The percent of positive cells after 2 h of incubation with different polymeric nanocapsules is depicted in FIG. 11 for 3 replicates. The percent of positive cells was around 40 to 55% for all the FITC-IgG-loaded nanocapsules, which represents a good capacity of the nanocapsules to interact with the cells in a short period of time (2 h).

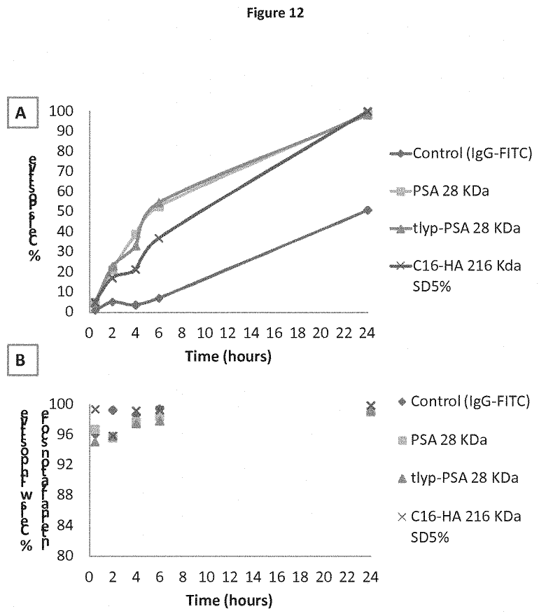

Second, an additional study was performed with a more advanced Imaging Flow Cytometer (ImageStream®) to know the ability of the nanocapsules to elicit an effective internalization of the associated antibody into the cells. Briefly, FITC-IgG-loaded nanocapules were incubated in 6-well plates with A549 cells (1 mL DMEM with 6 mg/mL nanocapsules/well), using separate wells per each time point to be studied (e.g. 0, 30 min, 2 h, 4 h, 6 h and 24 h). At each predetermined timepoint the cells were trypsinized and the images were acquired in the ImageStream® device to determine the percent of positive cells for the nanocapsules (FIG. 12A) and the corresponding FITC-IgG internalization score (FIG. 12B). The effective internalization was determined by labeling cytoplasm acidic organelles with Lysotracker® fluorescent marker for live cells, and further confirmed by confocal microscopy (data not shown).

As can be observed in FIG. 12, FITC-IgG-loaded PSA, PSA-tLyp1 and C16-HA216 SD5% nanocapsules, elicited an effective internalization of the associated antibody model into the cells in a time-dependent manner, up to a 100% of positive cells.

This example thus illustrates the potential of polymeric nanocapsules to promote the cell internalization of the associated mAbs.

### Example 13

This example illustrates the possibility of associating two actives of very different nature and size in the same nanocapsule. As a non-limiting example, C16-HA 216 SD 5%, C16-HA 55 SD 7%-tlyp nanocapsules, and PSA nanocapsules were formulated with both the mAb Bevacizumab (hydrosoluble macromolecule) and Paclitaxel (liposoluble small molecule) by a self-emulsifying technique.

Briefly, the oily phase was prepared by weighting 290 mg of Polysorbate 80 (Tween 80®, Merck), 295 mg of caprylic/capric triglycerides (Labrafac Lipophile WL 1349®, Gattefose), 12.5 mg of Kolliphor HS15 (BASF) and 5 mg of paclitaxel in a glass vial, agitating all the components under magnetic stirring at 700 rpm to completely mix and solubilize them. In the case of PSA nanocapsules, the oily phase additionally contained benzethonium chloride, as previously reported in the Example 8 for non-amphiphilic polymers.

In parallel, the aqueous phase was prepared by solubilizing the polymers, separately, in PBS pH 7.3 (25 mM) (0.25 mg/ml for C16-HA-based nanocapsules and 3 mg/mL for PSA nanocapsules) and adding the corresponding amount of Bevacizumab for a final concentration in the formulation of 0.5 mg/ml. After that, 4.415 mL of aqueous phase was added over the oily phase (597.5 mg) under magnetic stirring (1250 rpm, 10 min).

The quantification of the drug was performed by HPLC. The HPLC system included a VWR Hitachi ELITE LaChrom (Hitachi, Tokyo, Japan) and a column compartment ACE Equivalence reversed-phase C-18 (5 micrometers×250 mm×4.6 mm; Aberdeen, Scotland). The experimental analytical conditions were as follows: the mobile phase included MilliQ water (A) and acetonitrile (B). An isocratic program 40% A and 60% B acidified with trifluoroacetic acid at 0.1% was used. The flow rate was 1.5 ml/min and the run time was 10.0 min. The temperature of the column was maintained at 30° C., the injected volume was 25 microliters and the UV detector in 227 nm. Under these conditions, PCX was eluted at 4.21+/−0.02 min.

The measurements of size, PDI, zeta potential, and associated mAb were done in the same way as previously described for mAb-loaded nanocapsules (Example 8). The total amount of mAb and palitaxel were analyzed in non-isolated nanocapsules samples by the corresponding ELISA and HLPC method, respectively. Results are shown in Table 21.

TABLE 21

| | | | | Total | Total | |
|---|---|---|---|---|---|---|
| | | | | Paclitaxel | Bevacizum | Bevacizumab |
| | | | Zeta | Content | ab content | associated |
| Formulation | Size | PDI | Potential | (%) | (%) | (%) |
| C16-HA 216 SD5% | 126 ± 2 | 0.27 | −11 ± 1 | 111.7 ± 2.6 | 116.4 ± 4.5 | 57.9 ± 7.3 |
| C16-HA(55 KDa | 145 ± 5 | 0.28 | −12 ± 2 | 106.7 ± 10.9 | 106.2 ± 10.8 | 71.9 ± 5.6 |
| SD7%)- tlyp | | | | | | |
| PSA | 154 ± 1 | 0.21 | −9 ± 2 | 110.4 ± 6.5 | 111.2 ± 8.7 | 64.0 ± 8.0 |

### REFERENCE LIST

Curr. Org. Chem., 17(9):975-998, 2013.
Giorgi, et al., “Carbohydrate PEGylation, an approach to improve pharmacological potency,” Beilstein J. Org. Chem., 10:1433-44, 2014.
Bertrand N., et al., Cancer Nanotechnology: The impact of passive and active targeting in the era of modern cancer biology, Advanced Drug Delivery Reviews 66 (2014) 2-25.
Gilad Y., et al., Recent innovations in peptide based targeted delivery to cancer cells, Biomedicines, 4 (2016).
Zhou G., et al. Aptamers: A promising chemical antibody for cancer therapy, Oncotarget, 7 (2016) 13446-13463.
Zhang D. et al., Cell-penetrating peptides as noninvasive transmembrane vectors for the development of novel multifunctional drug-delivery systems, Journal of Controlled Release, Volume 229 (2016) Pages 130-139.
Regberg J., et al. Applications of cell-penetrating peptides for tumor targeting and future cancer therapies, Pharmaceuticals, 5 (2012) 991-1007.
Ruoslahti E., Tumor penetrating peptides for improved drug delivery, Advanced Drug Delivery Reviews, Volumes 110-111 (2017) Pages 3-12.
Mojarradi, “Coupling of substances containing a primary amine to hyaluronan via carbodiimide-mediated amidation,” Master's Thesis, Uppsala University, March, 2011.

1. A composition, comprising:
a plurality of nanoentities comprising an inner portion surrounded by an outer shell, the outer shell comprising a polymer and a targeting moiety, the inner portion comprising at least one hydrophobic compound.

2. The composition according to claim 1, wherein the polymer is selected from the group consisting of:
polysialic acid (PSA),
hyaluronic acid (HA),
polyglutamic acid (PGA) and/or pegylated-polyglutamic acid (PGA-PEG),
polylactic acid (PLA) and/or pegylated polylactic acid (PLA-PGE),
poly(aspartic acid) (PASP) and/or pegylated-poly(aspartic acid) (PASP-PEG),
poly(lactic-co-glycolic acid) (PGLA) and/or pegylated poly(lactic-co-glycolic acid) (PGLA-PEG),
polyasparaginic acid and/or pegylated polyasparaginic acid,
alginic acid and/or pegylated alginic acid,
polymalic acid and/or pegylated polymalic acid, and mixtures thereof.

3. The composition according to claim 1, wherein the targeting moiety comprises a cell-penetrating peptide and/or a tumor/tis sue-penetrating peptide.

4. The composition according to claim 1, wherein the targeting moiety is selected from the group consisting of Lyp1, tLyp1, cLyp1, iNGR, iRGD, RPARPAR, TT1, linear TT1, RGD-4C, cRGD, Cilengitide, F3, 9-RGD, RGD4C, Delta 24-RGD, Delta 24-RGD4C, RGD-K5, acyclic RGD4C, bicyclic RGD4C, c(RGDfK), c(RGDyK), E-[c(RGDfK)₂], E[c(RGDyK)]₂, KLWVLPKGGGC, CDCRGDCFC, LABL, angiopeptin-2, antibodies, nanobodies, transferrin, ankyrin repeat protein, affibodies, folic acid, triphenylphosphonium, ACUPA, PSMA, carbohydrate moieties and aptamers.

5. The composition according to claim 4, wherein the targeting moiety comprises Lyp-1, tLyp, cLvp-1, or iRGD.

6. The composition according to claim 1, wherein the targeting moiety comprises a CendR peptide.

7-8. (canceled)

9. The composition according to claim 1, wherein at least some of the polymer is linked to a hydrophobic moiety.

10. The composition according to claim 9, wherein the hydrophobic moiety is selected from an alkyl group, cycloalkanes, bile salts and derivatives, terpenoids, terpenes, terpene-derived moieties and lipophilic vitamins.

11. The composition according to claim 10, wherein the hydrophobic moiety comprises a C₂-C₂₄ straight-chain alkyl group.

12. (canceled)

13. The composition according to claim 1, wherein at least about 90 wt % of the outer shell comprises a polymer.

14. The composition according to claim 1, wherein at least some of the plurality of nanoentities are nanocapsules with an average diameter of less than 1 micrometer.

15. (canceled)

16. The composition according to claim 1, wherein the polymer is polysialic acid.

17. The composition according to claim 1, wherein the targeting moiety is bonded to the polymer via an aminoalkyl (C₁-C₄) maleimide linker, an aminoalkyl (C₁-C₄) methacrylamide linker, or directly through an amide group.

18. (canceled)

19. The composition according to claim 17, wherein the targeting moiety is bonded to the polymer via an aminoethylmaleimide linker.

20. The composition according to claim 16, wherein the targeting moiety is bonded to the polymer via an aminoalkyl (C₁-C₄) succinimide linker, an aminoalkyl (C₁-C₄) amide-iso-propyl linker, or directly through an amide group.

21. (canceled)

22. The composition according to claim 20, wherein the targeting moiety is bonded to the polymer via an aminoethylsuccinimide linker.

23-28. (canceled)

29. A composition, comprising:
a plurality of nanoentities comprising an inner portion surrounded by an outer shell, the outer shell comprising a polymer, the inner portion comprising at least one hydrophobic compound, with the proviso that the at least about 90% of the polymer is not hyaluronic acid.

30. The composition according to claim 29, wherein the nanoentities comprise a pharmaceutical agent.

31. The composition according to claim 30, wherein the pharmaceutical agent is a monoclonal antibody.

32. (canceled)

33. The composition according to claim 30, wherein the pharmaceutical agent is contained in the inner portion of the nanoentities.

34. (canceled)

35. The composition according to claim 29, wherein the nanoentities comprise a monoclonal antibody and a small molecule having a molecular weight of less than 1000 Da.

36. The composition according to claim 1, wherein the nanoentities comprise a monoclonal antibody and a small molecule having a molecular weight of less than 1000 Da.

37. The composition according to claim 1, wherein the nanoentities comprise a pharmaceutical agent.

38. The composition according to claim 37, wherein the pharmaceutical agent is a monoclonal antibody.


SEQUENCE LISTING (22 sequences)

SEQ ID NO: 1 - protein, 9 aa, Artificial sequence
  Synthetic sequence
CGNKRTRGC

SEQ ID NO: 2 - protein, 7 aa, Artificial sequence
  Synthetic sequence
CGNKRTR

SEQ ID NO: 3 - protein, 29 aa, Artificial sequence
  Synthetic sequence
YTIWMPENPRPGTPCDIFTNSRGKRASNG

SEQ ID NO: 4 - protein, 9 aa, Artificial sequence
  Artificial sequence
CRNGRGPDC

SEQ ID NO: 5 - protein, 9 aa, Artificial sequence
  Synthetic sequence
CRGDKGPDC

SEQ ID NO: 6 - protein, 9 aa, Artificial sequence
  Synthetic sequence
CRGDRGPDC

SEQ ID NO: 7 - protein, 7 aa, Artificial sequence
  Synthetic sequence
RPARPAR

SEQ ID NO: 8 - protein, 9 aa, Artificial sequence
  Synthetic sequence
CKRGARSTC

SEQ ID NO: 9 - protein, 9 aa, Artificial sequence
  Synthetic sequence
AKRGARSTA

SEQ ID NO: 10 - protein, 4 aa, Artificial sequence
  Synthetic sequence
CRGD

SEQ ID NO: 11 - protein, 9 aa, Artificial sequence
  Synthetic sequence
CDCRGDCFC

SEQ ID NO: 12 - protein, 11 aa, Artificial sequence
  Synthetic sequence
LGASWHRPDKG

SEQ ID NO: 13 - protein, 5 aa, Artificial sequence
  Synthetic sequence
DEVDG

SEQ ID NO: 14 - protein, 6 aa, Artificial sequence
  Synthetic sequence
PLGLAG

SEQ ID NO: 15 - protein, 11 aa, Artificial sequence
  Synthetic sequence
KLWVLPKGGGC

SEQ ID NO: 16 - protein, 9 aa, Artificial sequence
  Synthetic sequence
CDCRGDCFC

SEQ ID NO: 17 - protein, 10 aa, Artificial sequence
  Synthetic sequence
IAGEDGDEFG

SEQ ID NO: 18 - protein, 9 aa, Artificial sequence
  Synthetic sequence
CPGPEGAGC

SEQ ID NO: 19 - protein, 5 aa, Artificial sequence
  Synthetic sequence
CREKA

SEQ ID NO: 20 - protein, 12 aa, Artificial sequence
  Synthetic sequence
RRRRRRRRGRGD

SEQ ID NO: 21 - protein, 5 aa, Artificial sequence
  Synthetic sequence
RGDYK

SEQ ID NO: 22 - protein, 31 aa, Artificial sequence
  Synthetic sequence
KDEPQRRSARLSAKPAPPKPEPKPKKAPAKK